SEQUENCE LISTING (41 sequences)

SEQ ID NO: 1 - DNA, 3180 bp, Rattus rattus
atgctctcagtggagaatggcctggaccctcgggctgccatccaggtcatcaagaagaagctagtgggatctgtgaaggccctgcagaaacagcatgtgtctctggacacagtggtcaccagtgaagatggagatgctaacaccatgtgcagtgccctggaggccgtgttcatccatggcttgcatgccaagtacatccgtgctgaggctggcggcaaaaggaagaagcacactcatcagaagcctctgcctcagcctgtcttctggccccttctgaaagctgtcacccacaaacacattatctcagacttggagcacctggtcttcatcaatacagacgtaggccgctgccgggcctggctgcggttggccctcaatgatgggctgatggagtgctacctgaagttgctcctgcaggaacctgcacggttgtgcgagtactatcagcccacggccctgcttcgagatgctgaggaagccgagttcctcctgagcttcctccaaggactaacatccttgtcttttgaactctcctacaagtcagccatcttaaatgaatggacactcaccccactgtctctctctgggctttgccccctctctgaactggaccctctcactgtctctggtgcagaactgcaacggaaggagtctctggattccatttcccattcctcaggctctgaggacatcgaagtccaacactcaggccataagatccgcagggacaggaagctcaccgcctcttccctcagcctggacacagccagctcatcccagctatcctgcagcctaaattctgacagctgcctgctccaagagaatggtcccaagagtccagaccactctgaggaacccatgtcctacgactcagatctgggcacagcaaatgctgatgactctgacaggtctctgcaagaggtgttgtcggaattcagcaaagcccaggtaaattctgcaccaagcagcggaccaagccaagagtcagacacccccatgttccagacccccctctccctccacagcctagctaacagcacacacctgctctttgaaggatctgaagagccctttcctgcccatacatcctctgggacttcaagtggccacaagcatcagccccaggagagtccagacatgcagccgctaggcactgcccaagctggccctgctgggagcacttcagaccagcagccatctagtcccgtggccggagcagctgaccaaggaagtgagccttggaaggccctggaatatggcagagttggaccgaagcttgtggtttcctctcctacgagtccgaaaggcaagagctggatttctgaagatgacttctgccgacctcccaaggagcatgctctgaagaacacttcagatctttgcatatctcctctgcaagggactccagagttgaggactgctctccacgggccgttttctcaaggaccaagaaagagctgctccctgggggccttagacaaagcgtgtgtgtcctcgctggactacagaaatgcccagacagctccatctccagcggtgacacaagaccataaaaacttctgtgtggtgcatcgaaggcagatggggctctctaacccattccggggcctcatgaagctgggcacagtagcccggcgaggagccatgggcatctggaaggaattcttttgtgagctgtccccactggagctccgcctctacctgagtgacgaggagcgcacctgtgtggagagctgctccctgctacggtgtgaggccgtgggaccagcacacagcgatggacgttttgagttggtcttctccggcaagaagctggctctccgtgcctcctcccaggacgaggctgaggactggcttgaccgtgtgcgggaggctctgcagaaggtgcgccctcagcaggaggaggagtgggtgaatatccagtacccagaccaggctgaggattccccagaggcgccaccagacaaccttcccccctattcagccctgctcccagaacatgcaggtgcacagggaatacagccaaactggacatctgcccaggttccagagccagatgctatcaaagagtctcttttgtacctgtatgcagaccggacctggattccctatatcttttccctgtccttggagtctctgaaatgtttccgtgtgagaaacaatgagaagatgctaagtgatagccatggagtggagaccatccgggacatcctgccagacaccagccttgggggcccagccttcttcaaaatcatcacggccaaggctgtcctcaaactgcaggccaagaacactgaggaggccgcccactggagagacctagtcaggaaagtgctggcgtcttacttagagtcagttcaggaggctgtgacacttgctgggagtctggatgaaaactgtcaggaggtcctgaagtttgccaccagggagaatggcttcctgctacagtaccttgtggccatcccgaccgagaagggcctcgactcccagggctgcttctgtgcaggttgctcccggcagatcggcttctcctttgtacgacctaagctctgtgccttctctggcctatattactgtgacttctgccaccaagacgatgcctcggtgattccagccagaatcattcataactgggacctcacaaagcgcccggtctgccggcaggccctgaagttcctggcccagatccgggcccagcccctcatcaacctgcagctggtgaacgcctctctgtatgagcatgtagagcgcatgcacctcattggcaggagccgggagcagctgaaacttctgggggactacctgggcctgtgccgaagtggtgctctgaaggagttgagcaaaaggctaagccacaggaattacctcttggagtctcctcacaaattcagtgttgctgacctccagcagattgcagagggggtgtatgaaggattcctcaaagccctgattgaattcgcctcccagcatgtctaccactgtgacctgtgcacccagcgaggcttcatctgccagatctgccaccaccaggacatcatctttccctttgagtttgacaccacagtcaggtgtgctgaatgcagaactgtcttccatcagagctgccaggctgtggtgaggaagggctgtccccgctgtgcccgccggcgcaagtaccaggaacagaacactgtcagttag

SEQ ID NO: 2 - protein, 1059 aa, Rattus rattus
MLSVENGLDPRAAIQVIKKKLVGSVKALQKQHVSLDTVVTSEDGDANTMCSALEAVFIHGLHAKYIRAEAGGKRKKHTHQKPLPQPVFWPLLKAVTHKHIISDLEHLVFINTDVGRCRAWLRLALNDGLMECYLKLLLQEPARLCEYYQPTALLRDAEEAEFLLSFLQGLTSLSFELSYKSAILNEWTLTPLSLSGLCPLSELDPLTVSGAELQRKESLDSISHSSGSEDIEVQHSGHKIRRDRKLTASSLSLDTASSSQLSCSLNSDSCLLQENGPKSPDHSEEPMSYDSDLGTANADDSDRSLQEVLSEFSKAQVNSAPSSGPSQESDTPMFQTPLSLHSLANSTHLLFEGSEEPFPAHTSSGTSSGHKHQPQESPDMQPLGTAQAGPAGSTSDQQPSSPVAGAADQGSEPWKALEYGRVGPKLVVSSPTSPKGKSWISEDDFCRPPKEHALKNTSDLCISPLQGTPELRTALHGPFSQGPRKSCSLGALDKACVSSLDYRNAQTAPSPAVTQDHKNFCVVHRRQMGLSNPFRGLMKLGTVARRGAMGIWKEFFCELSPLELRLYLSDEERTCVESCSLLRCEAVGPAHSDGRFELVFSGKKLALRASSQDEAEDWLDRVREALQKVRPQQEEEWVNIQYPDQAEDSPEAPPDNLPPYSALLPEHAGAQGIQPNWTSAQVPEPDAIKESLLYLYADRTWIPYIFSLSLESLKCFRVRNNEKMLSDSHGVETIRDILPDTSLGGPAFFKIITAKAVLKLQAKNTEEAAHWRDLVRKVLASYLESVQEAVTLAGSLDENCQEVLKFATRENGFLLQYLVAIPTEKGLDSQGCFCAGCSRQIGFSFVRPKLCAFSGLYYCDFCHQDDASVIPARIIHNWDLTKRPVCRQALKFLAQIRAQPLINLQLVNASLYEHVERMHLIGRSREQLKLLGDYLGLCRSGALKELSKRLSHRNYLLESPHKFSVADLQQIAEGVYEGFLKALIEFASQHVYHCDLCTQRGFICQICHHQDIIFPFEFDTTVRCAECRTVFHQSCQAVVRKGCPRCARRRKYQEQNTVS

SEQ ID NO: 3 - DNA, 3179 bp, Rattus rattus
atgctctcagtggagaatggcctggaccctcgggctgccatccaggtcatcaagaagaagctagtgggatctgtgaaggccctgcagaaacagcatgtgtctctggacacagtggtcaccagtgaagatggagatgctaacaccatgtgcagtgccctggaggccgtgttcatccatggcttgcatgccaagtacatccgtgctgaggctggcggcaaaaggaagaagcacactcatcagaagcctctgcctcagcctgtcttctggccccttctgaaagctgtcacccacaaacacattatctcagacttggagcacctggtcttcatcaatacagacgtaggccgctgccgggcctggctgcggttggccctcaatgatgggctgatggagtgctacctgaagttgctcctgcaggaacctgcacggttgtgcgagtactatcagcccacggccctgcttcgagatgctgaggaagccgagttcctcctgagcttcctccaaggactaacatccttgtcttttgaactctcctacaagtcagccatcttaaatgaatggacactcaccccactgtctctctctgggctttgccccctctctgaactggaccctctcactgtctctggtgcagaactgcaacggaaggagtctctggattccatttcccattcctcaggctctgaggacatcgaagtccaacactcaggccataagatccgcagggacaggaagctcaccgcctcttccctcagcctggacacagccagctcatcccagctatcctgcagcctaaattctgacagctgcctgctccaagagaatggtcccaagagtccagaccactctgaggaacccatgtcctacgactcagatctgggcacagcaaatgctgatgactctgacaggtctctgcaagaggtgttgtcggaattcagcaaagcccaggtaaattctgcaccaagcagcggaccaagccaagagtcagacacccccatgttccagaccccctctccctccacagcctagctaacagcacacacctgctctttgaaggatctgaagagccctttcctgcccatacatcctctgggacttcaagtggccacaagcatcagccccaggagagtccagacatgcagccgctaggcactgcccaagctggccctgctgggagcacttcagaccagcagccatctagtcccgtggccggagcagctgaccaaggaagtgagccttggaaggccctggaatatggcagagttggaccgaagcttgtggtttcctctcctacgagtccgaaaggcaagagctggatttctgaagatgacttctgccgacctcccaaggagcatgctctgaagaacacttcagatctttgcatatctcctctgcaagggactccagagttgaggactgctctccacgggccgttttctcaaggaccaagaaagagctgctccctgggggccttagacaaagcgtgtgtgtcctcgctggactacagaaatgcccagacagctccatctccagcggtgacacaagaccataaaaacttctgtgtggtgcatcgaaggcagatggggctctctaacccattccggggcctcatgaagctgggcacagtagcccggcgaggagccatgggcatctggaaggaattcttttgtgagctgtccccactggagctccgcctctacctgagtgacgaggagcgcacctgtgtggagagctgctccctgctacggtgtgaggccgtgggaccagcacacagcgatggacgttttgagttggtcttctccggcaagaagctggctctccgtgcctcctcccaggacgaggctgaggactggcttgaccgtgtgcgggaggctctgcagaaggtgcgccctcagcaggaggaggagtgggtgaatatccagtacccagaccaggctgaggattccccagaggcgccaccagacaaccttcccccctattcagccctgctcccagaacatgcaggtgcacagggaatacagccaaactggacatctgcccaggttccagagccagatgctatcaaagagtctcttttgtacctgtatgcagaccggacctggattccctatatcttttccctgtccttggagtctctgaaatgtttccgtgtgagaaacaatgagaagatgctaagtgatagccatggagtggagaccatccgggacatcctgccagacaccagccttgggggcccagccttcttcaaaatcatcacggccaaggctgtcctcaaactgcaggccaagaacactgaggaggccgcccactggagagacctagtcaggaaagtgctggcgtcttacttagagtcagttcaggaggctgtgacacttgctgggagtctggatgaaaactgtcaggaggtcctgaagtttgccaccagggagaatggcttcctgctacagtaccttgtggccatcccgaccgagaagggcctcgactcccagggctgcttctgtgcaggttgctcccggcagatcggcttctcctttgtacgacctaagctctgtgccttctctggcctatattactgtgacttctgccaccaagacgatgcctcggtgattccagccagaatcattcataactgggacctcacaaagcgcccggtctgccggcaggccctgaagttcctggcccagatccgggcccagcccctcatcaacctgcagctggtgaacgcctctctgtatgagcatgtagagcgcatgcacctcattggcaggagccgggagcagctgaaacttctgggggactacctgggcctgtgccgaagtggtgctctgaaggagttgagcaaaaggctaagccacaggaattacctcttggagtctcctcacaaattcagtgttgctgacctccagcagattgcagagggggtgtatgaaggattcctcaaagccctgattgaattcgcctcccagcatgtctaccactgtgacctgtgcacccagcgaggcttcatctgccagatctgccaccaccaggacatcatctttccctttgagtttgacaccacagtcaggtgtgctgaatgcagaactgtcttccatcagagctgccaggctgtggtgaggaagggctgtccccgctgtgcccgccggcgcaagtaccaggaacagaacactgtcagttag

SEQ ID NO: 4 - protein, 342 aa, Rattus rattus
MLSVENGLDPRAAIQVIKKKLVGSVKALQKQHVSLDTVVTSEDGDANTMCSALEAVFIHGLHAKYIRAEAGGKRKKHTHQKPLPQPVFWPLLKAVTHKHIISDLEHLVFINTDVGRCRAWLRLALNDGLMECYLKLLLQEPARLCEYYQPTALLRDAEEAEFLLSFLQGLTSLSFELSYKSAILNEWTLTPLSLSGLCPLSELDPLTVSGAELQRKESLDSISHSSGSEDIEVQHSGHKIRRDRKLTASSLSLDTASSSQLSCSLNSDSCLLQENGPKSPDHSEEPMSYDSDLGTANADDSDRSLQEVLSEFSKAQVNSAPSSGPSQESDTPMFQTPSPSTA

SEQ ID NO: 5 - DNA, 3225 bp, Mus musculus
atgctctcagtggagaatggcctggaccctcgtgctgccatccaggtcatcaagaagaagctcgtgggatccgtgaaggctctgcagaaacagcatgtgtctctggacacagtggtcaccagtgaagatggagatgccaacaccatgtgcagtgccctggaggctgtgttcatccacggcctgcatgccaagcacatccgcgccgaggctggcggcaaaaggaagaagcacactcaccagaaggctctgcctcagcctgtcttctggccccttctgaaagccatcacccacagacacattgtctcagatttggagcacctggtcttcatcaacacagatgtaggccgttgccgagcctggctgcggttggccctcaatgacggtctgatggagtgctatctgaaactgctcctgcaggaacctgcacggttgtgcgagtactatcagcccacagccctgcttcgagatgccgaggaggctgagttcctcctgagcttcctgcaaggactaacatccttgtccttcgaactctcctacaagtcagccatcttaaatgaatggacactcaccccattgtcgctctctgggctttgccccctctctgaactcgaccctctcactacctctggtgcagaactacagcggaaagagtctctggattcaatttcccattcctcgggctctgaggacatcgaagtccaacactcaggccataagatccgcaggaacaggaagctcactgcctcttccctcagcctggacacagccagttcatcccagctgtcctgcagcctaaattctgacagctgcctgctccaagagaatggtcccaagagtccagaccattctgaggaacccatgtcctatgactcagatctgggcatggcaaacaccgatgaccccgacaggtctctgcaagaggtgttgtcggaattcagcaaagcccaggtaaattctgcaccaagcagcggaccaaaccaagagccagacacccccatgttccagacgcccctctccctccatagcctagctaccagcacacacctgcactttgagggatctgaagagctctttcctgcccacaaatcctctgggacatcaagtggtggccataagcatcagctactgccccaggagaccccagacgagaagcagctaggcactgcccaagctggccctgcacagagcacttcagaccagcagccttctagtcctgtgggtggagcagctggccaagggagtggcccttggaaggccctggagtatggcagagtcggaccgaagcttgtggtttcctctcctacgagtccgaaaggcaagagctggatttctgaagatgacttctgccgacctccccaggagcctgctctaaagagcgctgcaggtctctgcacatctcctgtgcaagacactccagagtcaagggctgctctccacgggcctttttctcaaggaccaagaaagagctgttccttgggggcattagacaaagcatgtgtgccctcacaggcctgtggaaatgcccagccagctccagccccagctccagctccagccccagccccagccccagctccaggggtgacacaagaccataaaaacttctgtgtggtgcaccgaaggcaaatggggctgtccaacccattccggggcctcatgaagctgggcacagtggcccggcgaggagccatgggcatctggaaggaattcttttgtgagctgtccccgttggagttccgcctctacttgagtgatgaggaacgcacctgtgtggagagctgctccctgctccgatgtgaggctgtggggccagcacacagtgatggacgttttgagttggtcttctcgggcaagaagctggctctccgtgcctcctcccaagacgaggctgaggactggcttgaccgcgtacgggaggctctgcagaaggtgcgccctcagcaggaagatgagtgggtgaatatccagtacccagaccaggctgaggatgccccagaggctccaccagacagcctccccccctattcaactctactccccgagcctgcgggtgcccagggcatgcagttggactggacatctgcccaggttccagaaccagatgctatcaaagagtcccttttgtatctgtatgcagaccggacctgggtcccctatatcttttccctgtccttggagtctctgaaatgtttccgagtgagaaacaatgagaagatgctaagtgatagccatggagtggagaccatccgagacatcctgccagacaccagccttgggggcccagccttcttcaaaatcatcacggccaaggccgtcctgaaactgcaggccaaaaacaccgaggaggccacccactggagagacctggtccggaaagtgctggcatcttacctagagtcagctgaggaggctgtgacacttggtgggagtctggatgaaaaatgtcaggaggtgctgaagtttgctaccagggagaacggcttcctgctacagtaccttgtggccatcccgaccgagaagggcctcgactcccagggctgcttctgcgcaggctgctcccgacagatcggcttctcctttgtacgacccaagctctgtgccttctctggcctgtattactgtgacttctgccaccaagacgatgcctcagtgattccagccagaatcatccacaactgggacctcacgaagcgcccggtatgccggcaggccctgaagttcctggcccagatccgggcccaacctctcatcaacctgcagctggtgaacgcctctctgtatgagcatgtggaacgcatgcacctcattggcaggagccgggagcagctgaaacttctgggggactacctgggcctgtgccgaagtggtgctctgaaggagttgtgcaaaaggctgagccacaggaattatctcctggagtctccacacagattcagtgttgctgacctccagcagattgcagagggggtatatgaagggttcctcaaagccctgattgaattcgcatcccagcatgtctaccactgtgacctgtgcacccagcgaggcttcatctgccagatctgccaccaccaggacatcatctttccctttgagtttgacaccacagtcaggtgtgctgaatgcagaactgtcttccatcagagctgccaggctgtggtgaggaagggctgtccccgatgtgcccgccggcgcaagtaccaggaacagaacgttgtcagttaa

SEQ ID NO: 6 - protein, 1074 aa, Mus musculus
MLSVENGLDPRAAIQVIKKKLVGSVKALQKQHVSLDTVVTSEDGDANTMCSALEAVFIHGLHAKHIRAEAGGKRKKHTHQKALPQPVFWPLLKAITHRHIVSDLEHLVFINTDVGRCRAWLRLALNDGLMECYLKLLLQEPARLCEYYQPTALLRDAEEAEFLLSFLQGLTSLSFELSYKSAILNEWTLTPLSLSGLCPLSELDPLTTSGAELQRKESLDSISHSSGSEDIEVQHSGHKIRRNRKLTASSLSLDTASSSQLSCSLNSDSCLLQENGPKSPDHSEEPMSYDSDLGMANTDDPDRSLQEVLSEFSKAQVNSAPSSGPNQEPDTPMFQTPLSLHSLATSTHLHFEGSEELFPAHKSSGTSSGGHKHQLLPQETPDEKQLGTAQAGPAQSTSDQQPSSPVGGAAGQGSGPWKALEYGRVGPKLVVSSPTSPKGKSWISEDDFCRPPQEPALKSAAGLCTSPVQDTPESRAALHGPFSQGPRKSCSLGALDKACVPSQACGNAQPAPAPAPAPAPAPAPAPGVTQDHKNFCVVHRRQMGLSNPFRGLMKLGTVARRGAMGIWKEFFCELSPLEFRLYLSDEERTCVESCSLLRCEAVGPAHSDGRFELVFSGKKLALRASSQDEAEDWLDRVREALQKVRPQQEDEWVNIQYPDQAEDAPEAPPDSLPPYSTLLPEPAGAQGMQLDWTSAQVPEPDAIKESLLYLYADRTWVPYIFSLSLESLKCFRVRNNEKMLSDSHGVETIRDILPDTSLGGPAFFKIITAKAVLKLQAKNTEEATHWRDLVRKVLASYLESAEEAVTLGGSLDEKCQEVLKFATRENGFLLQYLVAIPTEKGLDSQGCFCAGCSRQIGFSFVRPKLCAFSGLYYCDFCHQDDASVIPARIIHNWDLTKRPVCRQALKFLAQIRAQPLINLQLVNASLYEHVERMHLIGRSREQLKLLGDYLGLCRSGALKELCKRLSHRNYLLESPHRFSVADLQQIAEGVYEGFLKALIEFASQHVYHCDLCTQRGFICQICHHQDIIFPFEFDTTVRCAECRTVFHQSCQAVVRKGCPRCARRRKYQEQNVVS

SEQ ID NO: 7 - DNA, 3171 bp, Homo sapiens
atgctttcagtggtggagaatggactggacccccaggctgccatcccggtcatcaagaagaagctggtgggatccgtgaaggccttgcagaagcagtacgtgtccctggacacggtggtcactagtgaagacggagatgccaacaccatgtgcagcgccctggaggccgtatttatccatggcctgcacgccaagcacatccgagctgaggccggaggaaaaaggaagaaaagtgcccaccagaagcctctgccccagcctgtcttctggcccctcctgaaagctgtcacccacaaacacatcatctcagagttggagcacctgacgtttgtcaacacggatgtgggccgctgccgggcatggctgcggctggccctgaacgatggcctgatggagtgctacctgaagctgctgctgcaggagcaggcccgcttgcatgagtactaccagcccaccgccctgctccgggatgctgaggagggcgagttcctccttagcttcctgcagggcctcacgtccttgtccttcgaactctcctacaagtctgccatcttaaatgagtggacgctcaccccattggccctgtctgggctttgcccgctttctgagctggaccctctctctacctctggtgcagaactacagcggaaggaatctctggattccatttcccattcttcaggctctgaagacatcgaagtccatcactcgggccataagatacggaggaaccagaagctgactgcctcctccctcagcctggacacggccagttcatcccagctgtcctgcagcctaaactctgatagctgcttactccaagagaatggctccaagagtccagaccattgcgaggagcccatgtcctgtgactcagacctgggcacagcaaatgctgaggactcagaccggtctctgcaagaggtattgttggaattcagcaaagcccaggtaaactctgtgccaaccaacggactgagccaagaaacagagatccccacaccacaggcctcgctctccctccatggcctcaacaccagcacatacctgcactgtgaggcacctgcagagccccttcctgcccaggcagcctctggaactcaagatggtgtccacgtgcaggagccgcgtccccaggcgcccagccccctggacttacagcagcctgtagagagcacctcaggccagcagccttctagtactgtcagcgagacagccagagaagtgggccaagggaatggcctgcagaaggcccaggctcatgacggagctggtctgaagctggtagtttcctcacccaccagtccgaaaaacaagagctggatctcagaggatgacttctaccggccttcccgggagcaacccctggagagtgcttcagaccacccaatagcttcttacagggggactccagggtcaaggcctggtctccacaggcatttttctcaagaaccaagaaaaaactgctccctgggggcgttagaccaagcgtgtgtaccttccccaggaagaaggcaagcccaggcagccccatcccaggggcataagagcttccgggtggtacaccggagacagatgggactgtccaacccattccggggtctcatgaagctgggcaccgtggagcggcggggggcaatgggcatctggaaggagctcttctgcgagctctccccgctggagttccgcctctacctgagcaacgaggagcacacctgtgtggagaactgctcgctgcttcgctgtgagtctgtggggccagcccatagtgatgggcgctttgagctggtcttctctggcaagaagctggccctgcgcgcctcctcccaggacgaagctgaggactggctggaccgggtgcgggaggccctgcagaaggtccggcctcagcaggaggatgagtgggtgaacgtgcagtacccagaccagcctgaggaaccccccgaggcgccccagggctgcctctctccctcagacctgctctcggagcccgcggccctccagggcacacagtttgactggtcgtccgcccaggttccagagccagatgccatcaaggagtccctgctgtacttgtacatggacaggacctggatgccctatatattttctctgtccttggaggctctgaaatgtttccgcatcaggaacaatgagaagatgctgagtgacagccacggcgtggagaccatccgggacatcctgccagacaccagccttgggggcccatccttcttcaaaatcatcacggccaaggctgtcctgaagctgcaggccggaaacgccgaggaagccgccctgtggagggatctggtccgcaaagtcctggcatcctacttggagacagccgaggaggcggtgaccctgggcgggagcctggatgaaaactgtcaggaggtgctgaaatttgccacccgggagaatggcttcctgctgcagtacctggtggctatccccatggagaaaggccttgactcccaaggctgcttctgcgcaggctgctcccggcagatcggcttctcctttgtacgacccaagctctgtgccttctctggcctctattactgtgacatctgccaccaagacgatgcctcagtgattccggccaggatcatccacaactgggacctcaccaagcgcccgatctgcaggcaggccctgaagtttctgacacagatccgggcccagcccctcatcaacctgcagatggtgaacgcgtctctgtacgagcatgtggagcggatgcacctcattgggaggagacgggagcagctgaagctcctgggggattacctgggcctgtgccggagtggcgccctgaaggagctcagcaagaggctcaaccacaggaattatctcttggaatctccgcataggttcagtgttgctgacctccaacagatcgcagacggggtgtatgaaggattcctcaaggccctgattgaatttgcctcccagcatgtctaccactgcgacctgtgcacccagcgcggcttcatctgccagatctgccagcaccacgacatcatcttcccctttgagtttgacaccacagtcaggtgtgccgagtgcaagaccgtcttccaccagagctgccaggctgtggtgaagaagggctgcccccgctgtgcccgccggcgcaagtaccaggaacagaacattttcgcctga

SEQ ID NO: 8 - protein, 1056 aa, Homo sapiens
MLSVVENGLDPQAAIPVIKKKLVGSVKALQKQYVSLDTVVTSEDGDANTMCSALEAVFIHGLHAKHIRAEAGGKRKKSAHQKPLPQPVFWPLLKAVTHKHIISELEHLTFVNTDVGRCRAWLRLALNDGLMECYLKLLLQEQARLHEYYQPTALLRDAEEGEFLLSFLQGLTSLSFELSYKSAILNEWTLTPLALSGLCPLSELDPLSTSGAELQRKESLDSISHSSGSEDIEVHHSGHKIRRNQKLTASSLSLDTASSSQLSCSLNSDSCLLQENGSKSPDHCEEPMSCDSDLGTANAEDSDRSLQEVLLEFSKAQVNSVPTNGLSQETEIPTPQASLSLHGLNTSTYLHCEAPAEPLPAQAASGTQDGVHVQEPRPQAPSPLDLQQPVESTSGQQPSSTVSETAREVGQGNGLQKAQAHDGAGLKLVVSSPTSPKNKSWISEDDFYRPSREQPLESASDHPIASYRGTPGSRPGLHRHFSQEPRKNCSLGALDQACVPSPGRRQAQAAPSQGHKSFRVVHRRQMGLSNPFRGLMKLGTVERRGAMGIWKELFCELSPLEFRLYLSNEEHTCVENCSLLRCESVGPAHSDGRFELVFSGKKLALRASSQDEAEDWLDRVREALQKVRPQQEDEWVNVQYPDQPEEPPEAPQGCLSPSDLLSEPAALQGTQFDWSSAQVPEPDAIKESLLYLYMDRTWMPYIFSLSLEALKCFRIRNNEKMLSDSHGVETIRDILPDTSLGGPSFFKIITAKAVLKLQAGNAEEAALWRDLVRKVLASYLETAEEAVTLGGSLDENCQEVLKFATRENGFLLQYLVAIPMEKGLDSQGCFCAGCSRQIGFSFVRPKLCAFSGLYYCDICHQDDASVIPARIIHNWDLTKRPICRQALKFLTQIRAQPLINLQMVNASLYEHVERMHLIGRRREQLKLLGDYLGLCRSGALKELSKRLNHRNYLLESPHRFSVADLQQIADGVYEGFLKALIEFASQHVYHCDLCTQRGFICQICQHHDIIFPFEFDTTVRCAECKTVFHQSCQAVVKKGCPRCARRRKYQEQNIFA

SEQ ID NO: 9 - DNA, 3266 bp, Homo sapiens
atgctttcagtggtggagaatggactggacccccaggctgccatcccggtcatcaagaagaagctggtgggatccgtgaaggccttgcagaagcagtacgtgtccctggacacggtggtcactagtgaagacggagatgccaacaccatgtgcagcgccctggaggccgtatttatccatggcctgcacgccaagcacatccgagctgaggccggaggaaaaaggaagaaaagtgcccaccagaagcctctgccccagcctgtcttctggcccctcctgaaagctgtcacccacaaatgagatttagctggagaggttttgctttgcggaggagcagcagactatgggacacttggcttttcctccccagctgttcaggaagccaacagagacacatcatctcagagttggagcacctgacgtttgtcaacacggatgtgggccgctgccgggcatggctgcggctggccctgaacgatggcctgatggagtgctacctgaagctgctgctgcaggagcaggcccgcttgcatgagtactaccagcccaccgccctgctccgggatgctgaggagggcgagttcctccttagcttcctgcagggcctcacgtccttgtccttcgaactctcctacaagtctgccatcttaaatgagtggacgctcaccccattggccctgtctgggctttgcccgctttctgagctggaccctctctctacctctggtgcagaactacagcggaaggaatctctggattccatttcccattcttcaggctctgaagacatcgaagtccatcactcgggccataagatacggaggaaccagaagctgactgcctcctccctcagcctggacacggccagttcatcccagctgtcctgcagcctaaactctgatagctgcttactccaagagaatggctccaagagtccagaccattgcgaggagcccatgtcctgtgactcagacctgggcacagcaaatgctgaggactcagaccggtctctgcaagaggtattgttggaattcagcaaagcccaggtaaactctgtgccaaccaacggactgagccaagaaacagagatccccacaccacaggcctcgctctccctccatggcctcaacaccagcacatacctgcactgtgaggcacctgcagagccccttcctgcccaggcagcctctggaactcaagatggtgtccacgtgcaggagccgcgtccccaggcgcccagccccctggacttacagcagcctgtagagagcacctcaggccagcagccttctagtactgtcagcgagacagccagagaagtgggccaagggaatggcctgcagaaggcccaggctcatgacggagctggtctgaagctggtagtttcctcacccaccagtccgaaaaacaagagctggatctcagaggatgacttctaccggccttcccgggagcaacccctggagagtgcttcagaccacccaatagcttcttacagggggactccagggtcaaggcctggtctccacaggcatttttctcaagaaccaagaaaaaactgctccctgggggcgttagaccaagcgtgtgtaccttccccaggaagaaggcaagcccaggcagccccatcccaggggcataagagcttccgggtggtacaccggagacagatgggactgtccaacccattccggggtctcatgaagctgggcaccgtggagcggcggggggcaatgggcatctggaaggagctcttctgcgagctctccccgctggagttccgcctctacctgagcaacgaggagcacacctgtgtggagaactgctcgctgcttcgctgtgagtctgtggggccagcccatagtgatgggcgctttgagctggtcttctctggcaagaagctggccctgcgcgcctcctcccaggacgaagctgaggactggctggaccgggtgcgggaggccctgcagaaggtccggcctcagcaggaggatgagtgggtgaacgtgcagtacccagaccagcctgaggaaccccccgaggcgccccagggctgcctctctccctcagacctgctctcggagcccgcggccctccagggcacacagtttgactggtcgtccgcccaggttccagagccagatgccatcaaggagtccctgctgtacttgtacatggacaggacctggatgccctatatattttctctgtccttggaggctctgaaatgtttccgcatcaggaacaatgagaagatgctgagtgacagccacggcgtggagaccatccgggacatcctgccagacaccagccttgggggcccatccttcttcaaaatcatcacggccaaggctgtcctgaagctgcaggccggaaacgccgaggaagccgccctgtggagggatctggtccgcaaagtcctggcatcctacttggagacagccgaggaggcggtgaccctgggcgggagcctggatgaaaactgtcaggaggtgctgaaatttgccacccgggagaatggcttcctgctgcagtacctggtggctatccccatggagaaaggccttgactcccaaggctgcttctgcgcaggctgctcccggcagatcggcttctcctttgtacgacccaagctctgtgccttctctggcctctattactgtgacatctgccaccaagacgatgcctcagtgattccggccaggatcatccacaactgggacctcaccaagcgcccgatctgcaggcaggccctgaagtttctgacacagatccgggcccagcccctcatcaacctgcagatggtgaacgcgtctctgtacgagcatgtggagcggatgcacctcattgggaggagacgggagcagctgaagctcctgggggattacctgggcctgtgccggagtggcgccctgaaggagctcagcaagaggctcaaccacaggaattatctcttggaatctccgcataggttcagtgttgctgacctccaacagatcgcagacggggtgtatgaaggattcctcaaggccctgattgaatttgcctcccagcatgtctaccactgcgacctgtgcacccagcgcggcttcatctgccagatctgccagcaccacgacatcatcttcccctttgagtttgacaccacagtcaggtgtgccgagtgcaagaccgtcttccaccagagctgccaggctgtggtgaagaagggctgcccccgctgtgcccgccggcgcaagtaccaggaacagaacattttcgcctga

SEQ ID NO: 10 - DNA, 3433 bp, Homo sapiens
atgctttcagtggtggagaatggactggacccccaggctgccatcccggtcatcaagaagaagctggtgggatccgtgaaggccttgcagaagcagtacgtgtccctggacacggtggtcactagtgaagacggagatgccaacaccatgtgcagcgccctggaggccgtatttatccatggcctgcacgccaagcacatccgagctgaggccggaggaaaaaggaagaaaagtgcccaccagaagcctctgccccagcctgtcttctggcccctcctgaaagctgtcacccacaaatgagatttagctggagaggttttgctttgcggaggagcagcagactatgggacacttggcttttcctccccagctgttcaggaagccaacagaggtgccctgtgttctagcatagagagagggtgaggtaggggttgaaagtgtcttctagaaaggcttatctctagaccaagcttcccagccagtgtgccagcagatcttgagaccctcagcactcaggcaagagcctgtggctgctttgagactggtcacaaaaagcagacacatcatctcagagttggagcacctgacgtttgtcaacacggatgtgggccgctgccgggcatggctgcggctggccctgaacgatggcctgatggagtgctacctgaagctgctgctgcaggagcaggcccgcttgcatgagtactaccagcccaccgccctgctccgggatgctgaggagggcgagttcctccttagcttcctgcagggcctcacgtccttgtccttcgaactctcctacaagtctgccatcttaaatgagtggacgctcaccccattggccctgtctgggctttgcccgctttctgagctggaccctctctctacctctggtgcagaactacagcggaaggaatctctggattccatttcccattcttcaggctctgaagacatcgaagtccatcactcgggccataagatacggaggaaccagaagctgactgcctcctccctcagcctggacacggccagttcatcccagctgtcctgcagcctaaactctgatagctgcttactccaagagaatggctccaagagtccagaccattgcgaggagcccatgtcctgtgactcagacctgggcacagcaaatgctgaggactcagaccggtctctgcaagaggtattgttggaattcagcaaagcccaggtaaactctgtgccaaccaacggactgagccaagaaacagagatccccacaccacaggcctcgctctccctccatggcctcaacaccagcacatacctgcactgtgaggcacctgcagagccccttcctgcccaggcagcctctggaactcaagatggtgtccacgtgcaggagccgcgtccccaggcgcccagccccctggacttacagcagcctgtagagagcacctcaggccagcagccttctagtactgtcagcgagacagccagagaagtgggccaagggaatggcctgcagaaggcccaggctcatgacggagctggtctgaagctggtagtttcctcacccaccagtccgaaaaacaagagctggatctcagaggatgacttctaccggccttcccgggagcaacccctggagagtgcttcagaccacccaatagcttcttacagggggactccagggtcaaggcctggtctccacaggcatttttctcaagaaccaagaaaaaactgctccctgggggcgttagaccaagcgtgtgtaccttccccaggaagaaggcaagcccaggcagccccatcccaggggcataagagcttccgggtggtacaccggagacagatgggactgtccaacccattccggggtctcatgaagctgggcaccgtggagcggcggggggcaatgggcatctggaaggagctcttctgcgagctctccccgctggagttccgcctctacctgagcaacgaggagcacacctgtgtggagaactgctcgctgcttcgctgtgagtctgtggggccagcccatagtgatgggcgctttgagctggtcttctctggcaagaagctggccctgcgcgcctcctcccaggacgaagctgaggactggctggaccgggtgcgggaggccctgcagaaggtccggcctcagcaggaggatgagtgggtgaacgtgcagtacccagaccagcctgaggaaccccccgaggcgccccagggctgcctctctccctcagacctgctctcggagcccgcggccctccagggcacacagtttgactggtcgtccgcccaggttccagagccagatgccatcaaggagtccctgctgtacttgtacatggacaggacctggatgccctatatattttctctgtccttggaggctctgaaatgtttccgcatcaggaacaatgagaagatgctgagtgacagccacggcgtggagaccatccgggacatcctgccagacaccagccttgggggcccatccttcttcaaaatcatcacggccaaggctgtcctgaagctgcaggccggaaacgccgaggaagccgccctgtggagggatctggtccgcaaagtcctggcatcctacttggagacagccgaggaggcggtgaccctgggcgggagcctggatgaaaactgtcaggaggtgctgaaatttgccacccgggagaatggcttcctgctgcagtacctggtggctatccccatggagaaaggccttgactcccaaggctgcttctgcgcaggctgctcccggcagatcggcttctcctttgtacgacccaagctctgtgccttctctggcctctattactgtgacatctgccaccaagacgatgcctcagtgattccggccaggatcatccacaactgggacctcaccaagcgcccgatctgcaggcaggccctgaagtttctgacacagatccgggcccagcccctcatcaacctgcagatggtgaacgcgtctctgtacgagcatgtggagcggatgcacctcattgggaggagacgggagcagctgaagctcctgggggattacctgggcctgtgccggagtggcgccctgaaggagctcagcaagaggctcaaccacaggaattatctcttggaatctccgcataggttcagtgttgctgacctccaacagatcgcagacggggtgtatgaaggattcctcaaggccctgattgaatttgcctcccagcatgtctaccactgcgacctgtgcacccagcgcggcttcatctgccagatctgccagcaccacgacatcatcttcccctttgagtttgacaccacagtcaggtgtgccgagtgcaagaccgtcttccaccagagctgccaggctgtggtgaagaagggctgcccccgctgtgcccgccggcgcaagtaccaggaacagaacattttcgcctga

SEQ ID NO: 11 - protein, 99 aa, Homo sapiens
MLSVVENGLDPQAAIPVIKKKLVGSVKALQKQYVSLDTVVTSEDGDANTMCSALEAVFIHGLHAKHIRAEAGGKRKKSAHQKPLPQPVFWPLLKAVTHK

SEQ ID NO: 12 - DNA, 11 bp, Homo sapiens
cacaagtgaga

SEQ ID NO: 13 - DNA, 11 bp, Homo sapiens
cacaaatgaga

SEQ ID NO: 14 - DNA, 20 bp, Artificial
  primer
agtaagagaggctgacctcc

SEQ ID NO: 15 - DNA, 21 bp, Artificial
  primer
acagtgcccaaggaagtgagc

SEQ ID NO: 16 - DNA, 20 bp, Artificial
  primer
ttgcacacgtatttggcacc

SEQ ID NO: 17 - DNA, 21 bp, Artificial
  primer
tctgttggcttcctgaacagc

SEQ ID NO: 18 - DNA, 22 bp, Artificial
  primer
ttattcctggttcctgctaacc

SEQ ID NO: 19 - DNA, 20 bp, Artificial
  primer
agtaacagacaatcacatgc

SEQ ID NO: 20 - DNA, 20 bp, Artificial
  primer
cagcagttttgcattcttcc

SEQ ID NO: 21 - DNA, 21 bp, Artificial
  primer
tgcggtcagacaagaacaacc

SEQ ID NO: 22 - DNA, 20 bp, Artificial
  primer
agctactcaggagactgggg

SEQ ID NO: 23 - DNA, 20 bp, Artificial
  primer
aacagaacttcacagctgcc

SEQ ID NO: 24 - DNA, 20 bp, Artificial
  primer
atgcactgcagggcatcagg

SEQ ID NO: 25 - DNA, 20 bp, Artificial
  primer
agtgagtagccagcctgccg

SEQ ID NO: 26 - DNA, 21 bp, Artificial
  primer
atcagacactgtgcacactcc

SEQ ID NO: 27 - DNA, 21 bp, Artificial
  primer
agctccccaacatcacagtgc

SEQ ID NO: 28 - DNA, 20 bp, Artificial
  primer
aagtgagcaggtcatgtggc

SEQ ID NO: 29 - DNA, 21 bp, Artificial
  primer
tgcatgtgcacgagtgcctgc

SEQ ID NO: 30 - DNA, 21 bp, Artificial
  primer
aggccgaattacactcctagc

SEQ ID NO: 31 - DNA, 21 bp, Artificial
  primer
tgcagaaaagctacagactgc

SEQ ID NO: 32 - DNA, 20 bp, Artificial
  primer
ccatggcacctcagtgcagc

SEQ ID NO: 33 - DNA, 21 bp, Artificial
  primer
atgggctgccccaacaactgc

SEQ ID NO: 34 - DNA, 19 bp, Artificial
  primer
gtgtgacgtatgggtaagc

SEQ ID NO: 35 - DNA, 22 bp, Artificial
  primer
taggcaaacccagccgggatgg

SEQ ID NO: 36 - DNA, 22 bp, Artificial
  primer
tccgtgaaggccttgcagaagc

SEQ ID NO: 37 - DNA, 24 bp, Artificial
  primer
tgggctggtagtactcatgcaagc

SEQ ID NO: 38 - DNA, 20 bp, Artificial
  primer
ctgttgtcttctttcccaac

SEQ ID NO: 39 - DNA, 20 bp, Artificial
  primer
agggcacacagtttgactgg

SEQ ID NO: 40 - DNA, 20 bp, Artificial
  primer
acatttcagagcctccaagg

SEQ ID NO: 41 - DNA, 11 bp, Homo sapiens
  misc_feature (6)..(6): n is a or g
cacaantgaga

(19) United States
(12) Patent Application Publication
(10) Pub. No.: US 2010/0034788 A1
(43) Pub. Date: Feb. 11, 2010

(54) Method for diagnosing and treating bone-related diseases

(76) Inventors: Wim Camiel Augusta Van Hul, Hever (BE); Liesbeth Louisa Hugo Van Wesenbeeck, Hoevenen (BE); Paul R. Odgren, Princeton, MA (US)

Correspondence Address: FISH & RICHARDSON PC, P.O. BOX 1022, MINNEAPOLIS, MN, 55440-1022, US

(21) Appl. No.: 11/665,811

(22) PCT Filed: Jul. 28, 2005
(86) PCT No.: PCT/EP05/08214

§ 371 Date: Apr. 19, 2007

(30) Foreign Application Priority Data
Jul. 28, 2004 (EP) PCT/EP04/08435

(51) Int. Cl. A61K 35/12; C12Q 1/68; A61K 31/7088; A61K 38/17

(52) U.S. Cl. 424/93.21; 435/6; 514/12

(57) ABSTRACT

The present invention relates to the field of molecular biology and medicine. The present invention relates to the use of polynucleotides encoding a mammalian PLEKHM1 polypeptide and the polypeptides encoded by these polynucleotides for regulating bone homeostasis and for diagnostic and/or therapeutic applications. The present invention further relates to vectors, host cells, antibodies, diagnostic and therapeutic methods for detecting and treating diseases, in particular bone-related diseases.

### FIELD OF THE INVENTION

The present invention relates to the field of molecular biology and medicine. The present invention relates to the use of polynucleotides encoding a mammalian PLEKHM1 polypeptide and the polypeptides encoded by these polynucleotides for regulating bone homeostasis and for diagnostic and/or therapeutic applications. The present invention further relates to vectors, host cells, antibodies, diagnostic and therapeutic methods for detecting and treating diseases, in particular bone-related diseases.

### BACKGROUND OF THE INVENTION

Bone diseases affect women, men, and children of all ages. From infancy to old age, bone disease profoundly alters the quality of life.

Bone homeostasis is dependent on two opposite and dynamic processes of bone formation and resorption in vertebrates and is regulated throughout adult life. Maintenance of skeletal integrity requires a dynamic balance between bone formation by osteoblasts and bone resorption by osteoclasts that is fine tuned by a network of systemic hormones and local factors. The role of the osteoclast is bone tissue resorption, a process that is counterbalanced by the osteoblast activity that results in bone tissue formation. Disturbance of this balance can lead to an extended number of skeletal pathologies, including major bone diseases such as osteoporosis and osteopetrosis.

The osteopetroses, a heterogeneous group of skeletal disorders, are characterized by an inadequate bone resorption resulting in an increased bone density (Benichou et al. 1998 Rev Rhum Engl Ed 65(12):778-87). More particularly, osteopetrosis results in accumulation of mineralized bone and cartilage due to a lack of bone remodeling activity. This activity is normally provided by osteoclast. Osteopetrosis results from a defect in the differentiation or the activation of the osteoclast. In humans, different forms of osteopetrosis are defined that can be classified based on the age of onset, severity and mode of inheritance. Currently, five different genes (grey-lethal, LRP5, CLCN7, TCIRG1 and CAII) have been identified to play a role in the pathogenesis of the different forms of human osteopetrosis (Chalhoub et al. 2003 Nat Med 9(4):399-406; Van Wesenbeeck et al. 2003 Am J Hum Genet 72(3):763-71; Campos-Xavier et al. 2003 Hum Genet 112(2):186-9; Cleiren et al. 2001 Hum Mol Genet 10(25):2861-7; Kornak et al. 2001 Cell 104(2):205-15; Frattini et al. 2000 Nat Genet 25(3):343-6; Kornak et al. 2000 Hum Mol Genet 9(13):2059-63 and Sly et al. 1983 Proc Natl Acad Sci USA 80(9):2752-6).

Osteoporosis is characterized by a decreased bone density and has the highest impact because of its high prevalence and its high incidence of fractures. Osteoporosis and related fractures arising from diminished bone density are particularly common in older individuals and contribute substantially to the healthcare costs and burden of illness associated with the disease. Although osteoporosis has many causes, about 80% of the underlying etiology is genetic. Unfortunately, there are no tests commercially available currently that can determine an individual's predisposition for osteoporosis. Very often, an individual is diagnosed with osteoporosis only after the disease has progressed extensively. Failure to provide early detection of bone disease and/or a predisposition for bone disease drives up the cost and suffering associated with such a disease.

In general, there remains a great need in the art for developing adequate diagnostic methods and diagnostic kits for detecting bone-related diseases and susceptibilities or predispositions thereto resulting from a disturbance in the bone homeostasis. There also remains a need in the art for adequate methods for preventing or treating bone-related diseases resulting from a disturbance in the bone homeostasis.

The present inventors studied the osteopetrotic phenotype in the incisors absent (ia) rat. Previously, the inventors reported localization of the gene responsible for the ia phenotype to a 4.7-cM region on rat chromosome 10q32.1 (Van Wesenbeeck et al. 2004 J Bone Miner Res. 19: 183-9; Van Wesenbeeck et al. 31^{st} European Symposium on Calcified Tissues, abstract P132, 5-9 Jun. 2004). However, this 4.7-cM region contained over 140 known or putatively assigned genes and it was not at all clear and could in no way be predicted at that time which gene would carry the ia mutation. Therefore, finding of the actual gene carrying the ia mutation required intense experimentation.

### SUMMARY

The present invention is based on the finding that a mammalian PLEKHM1 gene plays a role in the regulation of bone homeostasis. More in particular, the applicant has demonstrated that a mutation in the nucleic acid sequence of a mammalian PLEKHM1 gene and the loss of function of the corresponding protein resulted in a disturbance in the bone homeostasis. In particular, loss of function mutations in the rat and human PLEKHM1 gene caused an osteopetrosis phenotype characterised by impaired bone resorption. Therefore, in a first aspect, the present invention provides for the use of a nucleic acid encoding a mammalian PLEKHM1 polypeptide or the complement thereof for regulating bone homeostasis.

The term “regulating bone homeostasis” as used herein refers to the activity of maintaining a suitable balance between bone formation and bone resorption. Disruption of this balance may induce some serous bone-related diseases such as, but not limited to osteoporosis, osteopetroses, etc. The term encompasses, e.g., decreasing bone resorption in order to increase bone mass or alternatively, increasing bone resorption in order to decrease bone mass.

The term “nucleic acid encoding a mammalian PLEKHM1 polypeptide” refers to a nucleic acid which encodes an PLEKHM1 polypeptide in a mammalian species.

The human PLEKHM1 gene has been partially described as the AP162 gene in Hartel-Schenk S. et al. (Glycoconj J. 2001 November-December; 18(11-12):915-23) with a putative function in colon but without an indication of any role in bone metabolism. A role for this gene in bone metabolism and more specifically in osteoclasts was so far unknown.

The human PLEKHM1 gene has functional homologous genes in different organisms, including in rat and mouse. In rat, the PLEKHM1 gene is also referred to as LOC303584 gene. Sequence alignments demonstrated that the homolog of the PLEKHM1 gene of human in rat and mouse is highly conserved with a 83% similarity of cDNA sequence between human and rat, and 83% similarity of cDNA sequence between human and mouse, and 93% similarity of cDNA sequence between rat and mouse. cDNA sequences of rat, mouse and human PLEKHM1 are respectively represented by SEQ ID NO: 1; SEQ ID NO: 5; and SEQ ID NO: 7. In a preferred embodiment, the invention relates to the use of a nucleic acid comprising a DNA sequence as given in SEQ ID NO: 1, 5 or 7, or the complement thereof for regulating bone homeostasis.

In another aspect, the present invention relates to the use of a nucleic acid encoding mammalian PLEKHM1 polypeptide having a mutation in its nucleotide sequence or the complement thereof for regulating bone homeostasis. The applicant has shown that a deletion in a mammalian PLEKHM1 gene sequence results in a frameshift mutation yielding a highly truncated, dysfunctioned protein, and that this deletion may result in osteopetrosis. In a preferred embodiment, the invention relates to the use of a nucleic acid comprising a DNA sequence as given in SEQ ID NO: 3 or the complement thereof for regulating bone homeostasis. The DNA sequence as given in SEQ ID NO: 3 is a DNA sequence of a mutated rat PLEKHM1 (also named LOC303584) gene.

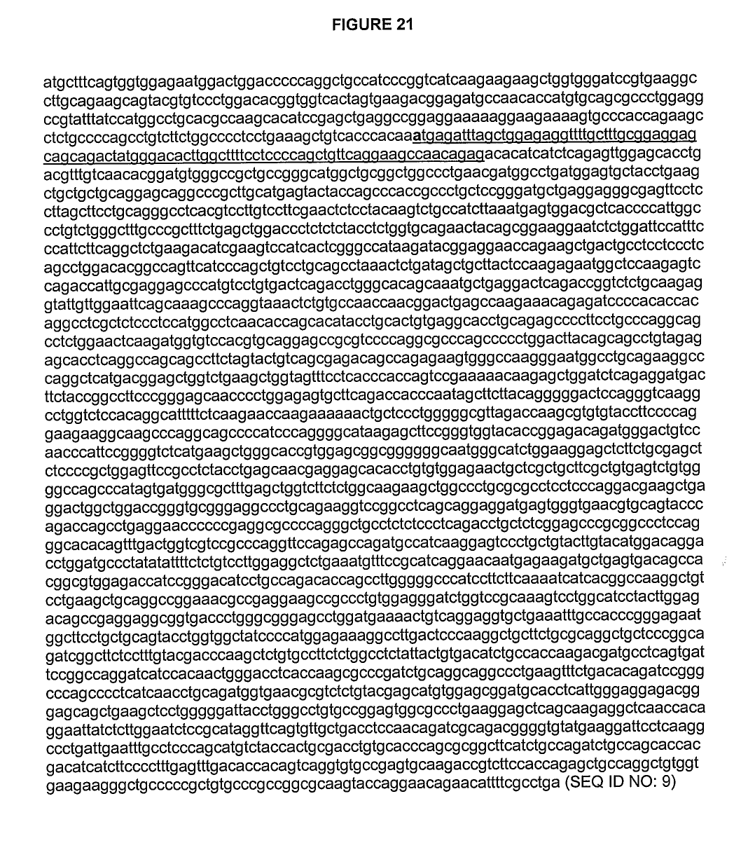

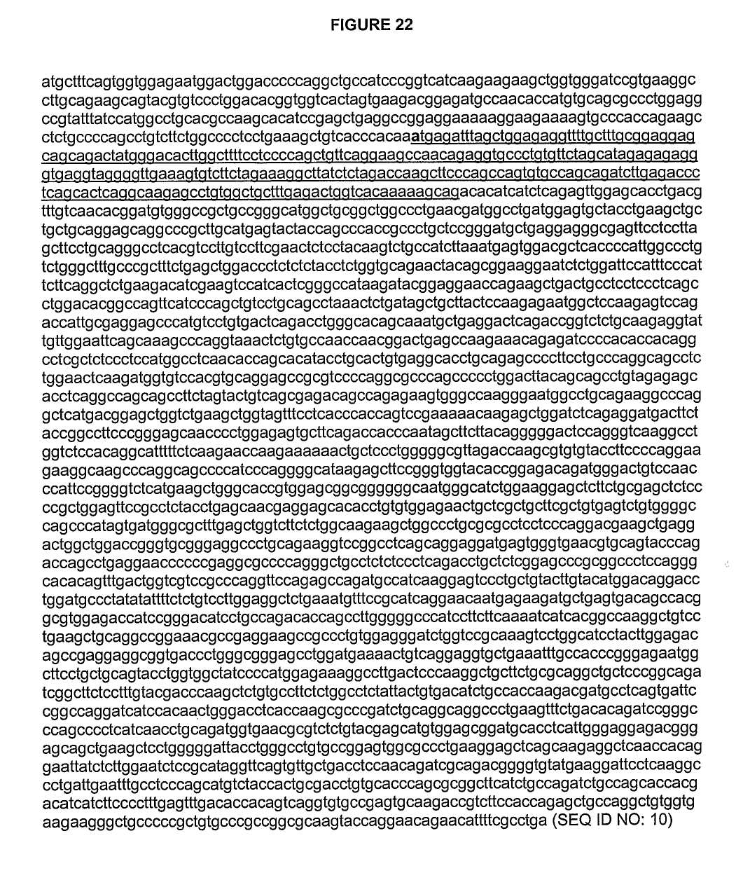

In addition, the applicant has shown that a single nucleotide mutation in the splice donor site of intron 2 of human PLEKHM1 gene (IVS2+1 G to A mutation) results in two alternative PLEKHM1 transcripts yielding a highly truncated and most likely dysfunctioned protein, and that this mutation results in osteopetrosis in homozygous state. In a preferred embodiment, the invention relates to the use of a nucleic acid comprising a DNA sequence as given in SEQ ID NO: 9 or SEQ ID NO: 10 or the complement thereof for regulating bone homeostasis. The DNA sequences given in SEQ ID NO: 9 and 10 (FIGS. 21 and 22) represent the two mutant human PLEKHM1 cDNA sequences resulting from the IVS2+1 G to A mutation.

The present invention further relates to vectors, host cells, antibodies, diagnostic and therapeutic methods for detecting and treating diseases, in particular bone-related diseases, involving the above defined polynucleotides and mutations thereof.

### DETAILED DESCRIPTION OF THE FIGURES

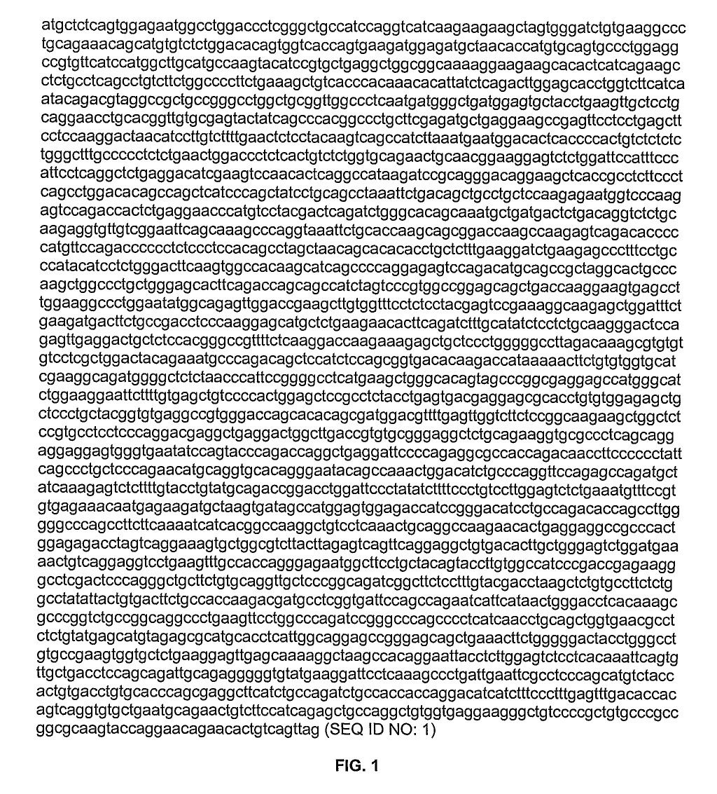

FIG. 1 represents the nucleic acid sequence of the rat PLEKHM1 gene (SEQ ID NO: 1—Genbank XM_{—}221013.2).

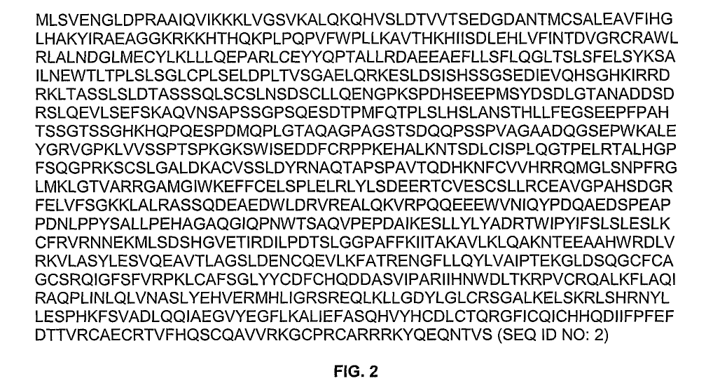

FIG. 2 represents the amino acid sequence of the rat PLEKHM1 polypeptide (SEQ ID NO: 2—Genbank XP_{—}221013.2).

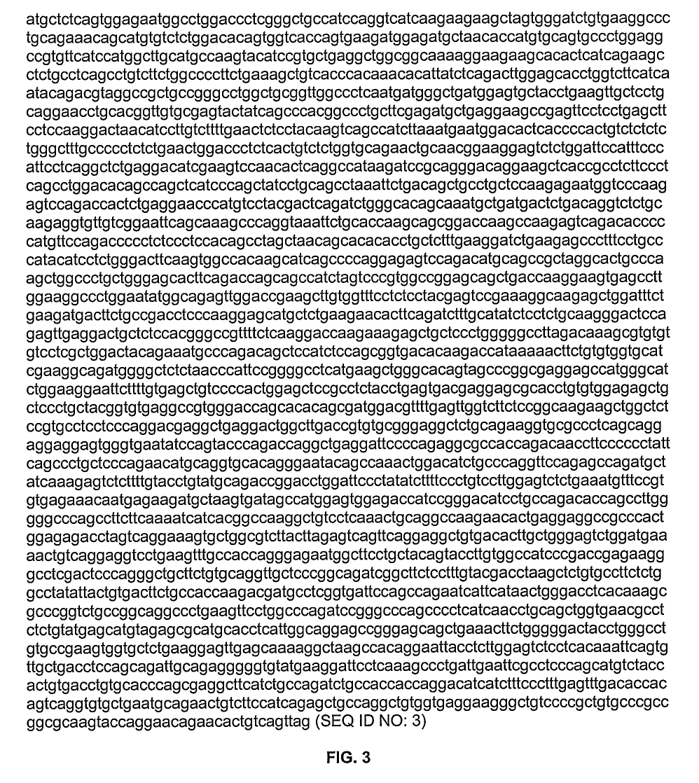

FIG. 3 represents the nucleic acid sequence of the mutated PLEKHM1 gene in ia rats (SEQ ID NO: 3).

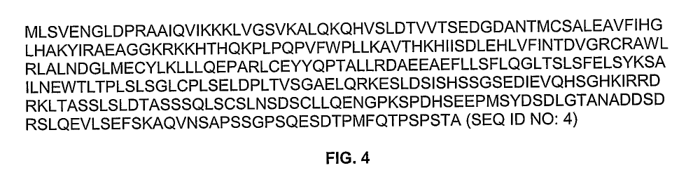

FIG. 4 represents the amino acid sequence of the corresponding mutated PLEKHM1 polypeptide in ia rats (SEQ ID NO: 4).

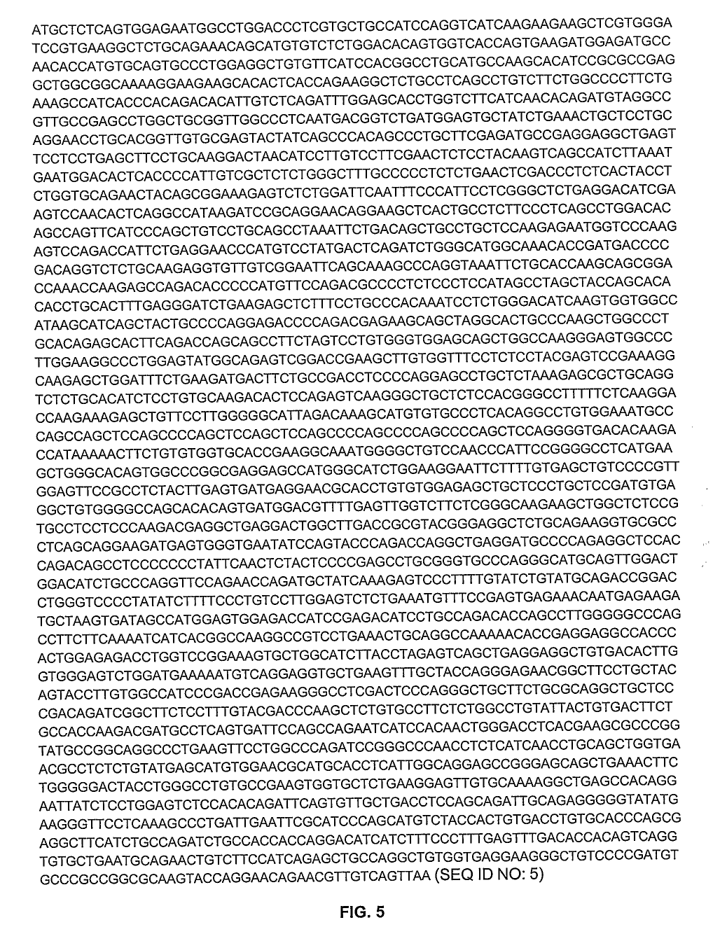

FIG. 5 represents the nucleic acid sequence of the mouse PLEKHM1 gene (SEQ ID NO: 5—Genbank NM_{—}183034.1).

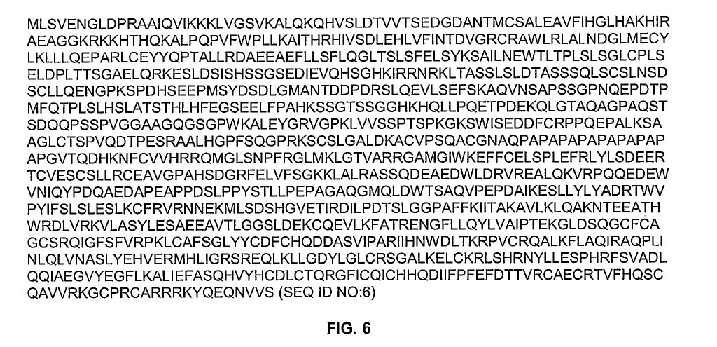

FIG. 6 represents the amino acid sequence of the mouse PLEKHM1 polypeptide (SEQ ID NO: 6—Genbank NP_{—}898855.1).

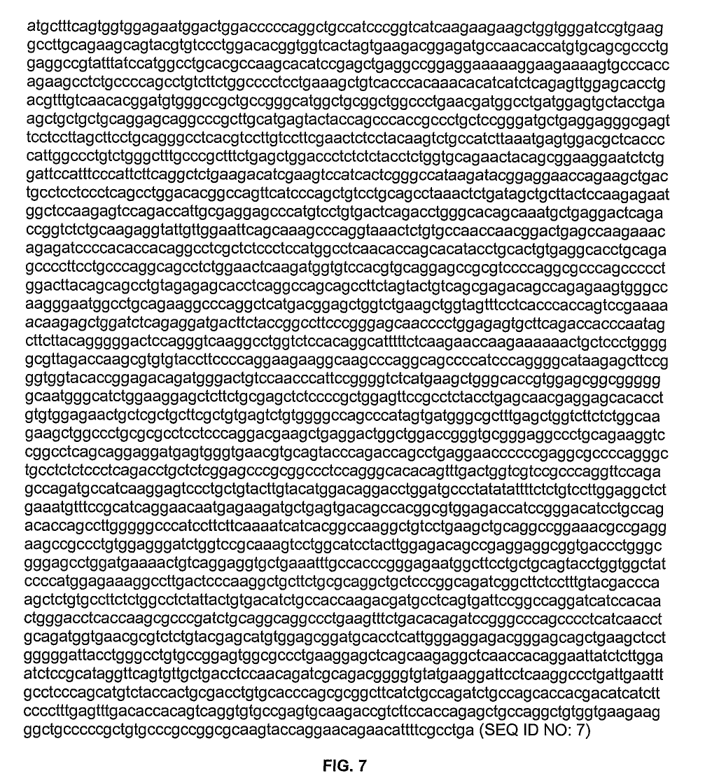

FIG. 7 represents the nucleic acid sequence of the human PLEKHM1 gene (SEQ ID NO: 7—Genbank NM_{—}014798.1).

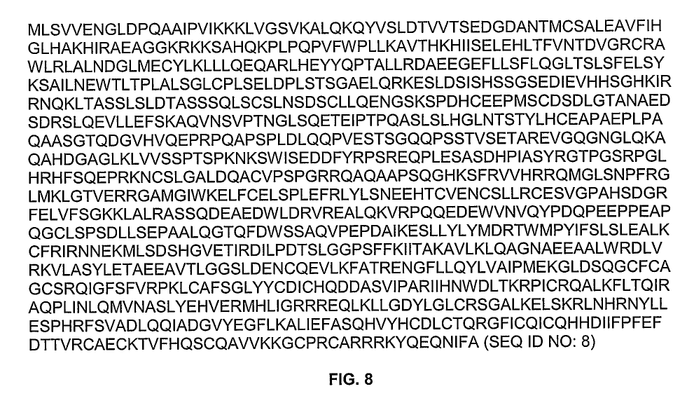

FIG. 8 represents the amino acid sequence of the human PLEKHM1 polypeptide (SEQ ID NO: 8—Genbank NP_{—}055613.1).

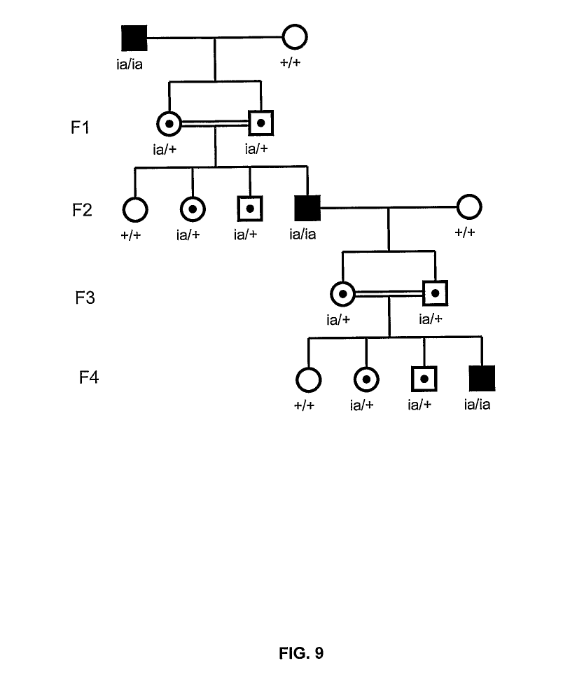

FIG. 9 represents a breeding scheme. Mutants are represented by filled symbols, wild types by open symbols and heterozygous mutants by spotted symbols. Co-segregation analysis was performed in F2 and F4 animals.

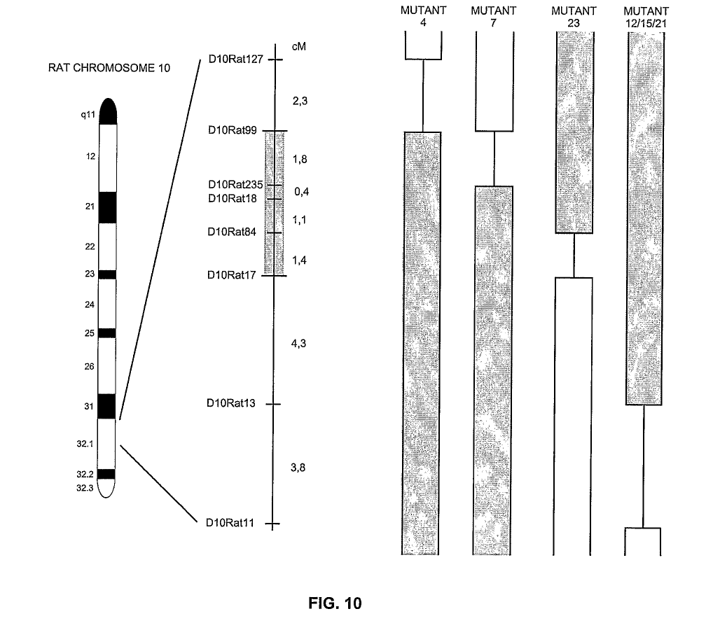

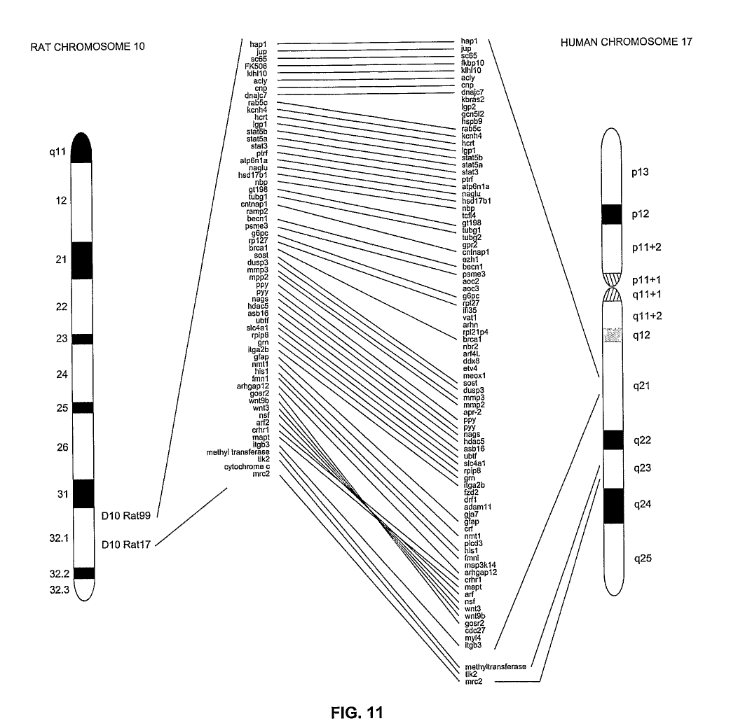

FIG. 10 illustrates an ideogram of rat chromosome 10 and genetic markers used to delineate the candidate region. The candidate region is shown as a grey box on the first vertical bar. Key recombinants are given on the other vertical lines. Gray bars represent chromosomal regions that may contain the disease causing gene, white bars indicate the regions that recombined and therefore cannot contain the disease gene. The lines in between represent uninformative regions.

FIG. 11 refers to homologous regions of rat chromosome 10q32.1 in human. The ia candidate region, flanked by the markers D10Rat99 and D10Rat17, on rat chromosome 10q32.1 is delineated. On the right, homology to human chromosome 17q21 and 17q23 is indicated. All the known genes in rat and human are listed and the homologous genes are connected with a line.

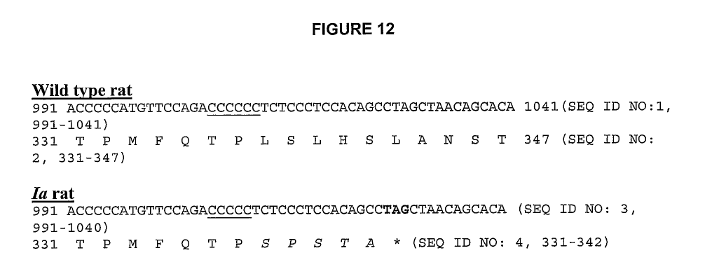

FIG. 12 illustrates the nucleotide and amino acid sequences of the ia mutation. A portion of the nucleotide sequence of the wild type rat plekhm1 gene (positions 991 to 1041 of SEQ ID NO: 1) is shown above together with its translation to the corresponding portion of the wild-type rat plekhm1 protein (positions 331 to 347 of SEQ ID NO: 2). A portion of the nucleotide sequence of the plekhm1 gene in ia rats (positions 991 to 1040 of SEQ ID NO: 3) around the position of the ia mutation is shown below together with its translation to the corresponding portion of the truncated ia rat plekhm1 protein (positions 331 to 342 of SEQ ID NO: 4). In the ia rats, a deletion of 1 cytosine (C) located in a stretch of 6 cytosines (underlined) at positions 1007 to 1012 of the wild-type plekhm1 nucleotide sequence (SEQ ID NO: 1) in exon 4 of the plekhm1 gene is observed. This deletion results in a frameshift mutation leading to 5 divergent amino acids followed by a stop codon (asterisk) in the truncated mutant plekhm1 protein in ia rats.

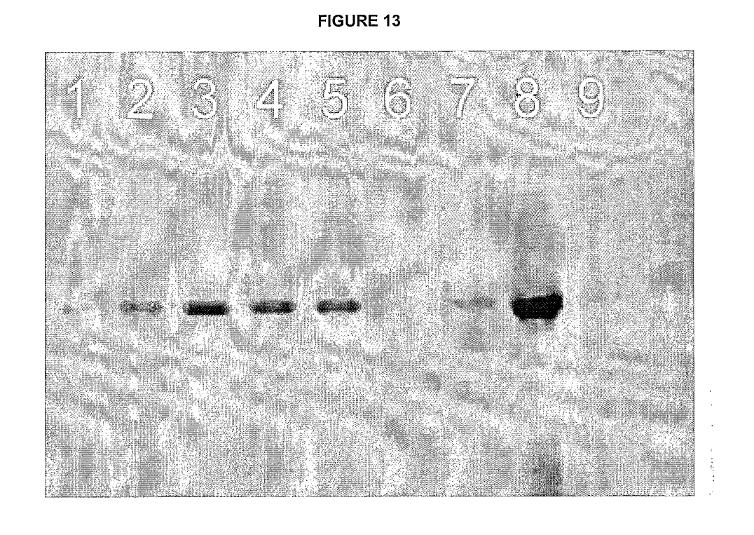

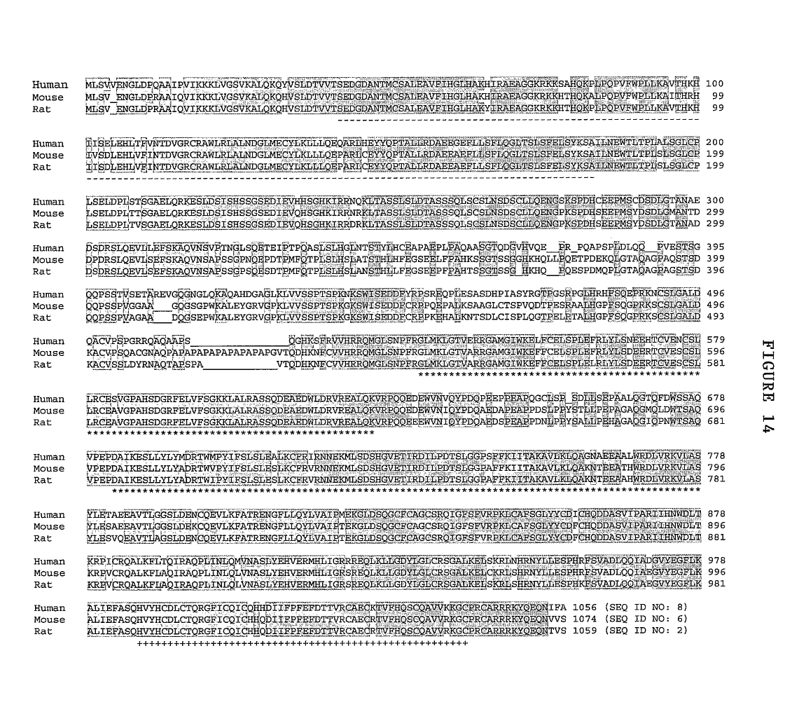

FIG. 13 illustrates the expression pattern of the plekhm1 gene using a multiple tissue rat cDNA panel; lanes 1-9 represent testis, skeletal muscle, lung, liver, spleen, brain, heart, kidney, and bone.

FIG. 14 illustrates alignment of the amino acid sequences of the human, mouse and rat PLEKHM1 protein; shaded residues are conserved between the three species; −−− indicates the RUN domain; *** indicates the PH domains; +++ indicates the cysteine-rich domain.

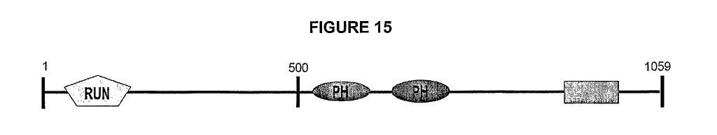

FIG. 15 illustrates the putative functional features of the rat plekhm1 protein predicted in silico using the InterPro database; predicted are one RUN domain (pentagon) and two PH domains (oval); further predicted is a functional domain (rectangle) which may be either a putative RING domain or a putative C1 domain, both of which are cystein rich domains.

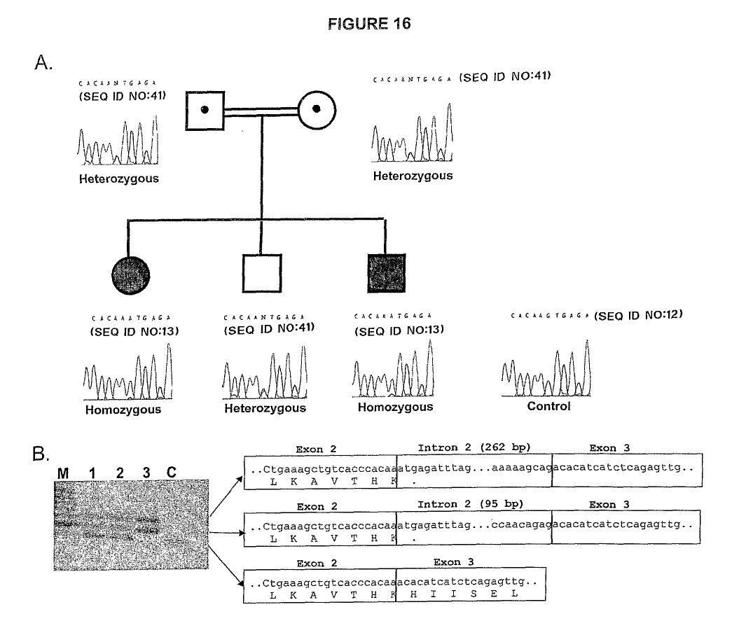

FIG. 16 illustrates the mutation analysis of a family with autosomal recessive osteopetrosis; (A) shows the pedigree and sequencing analysis of the IVS2+1 G to A mutation. The genomic sequence around the boundary between exon 2 and intron 2 in an unrelated control (wild-type sequence) is indicated by SEQ ID NO: 12 with the first nucleotide of intron 2 (G) at position 6 of SEQ ID NO: 12. The control was homozygous for the G allele. The two patients (filled symbols) were homozygous for this mutation (i.e., two A alleles) and thus produced the sequence around the boundary between exon 2 and intron 2 shown in SEQ ID NO: 13. The IVS2+1 G to A mutation is found at position 6 of SEQ ID NO: 13. The parents and one sibling were heterozygous for this mutation (i.e., one G and one A allele) and thus produced the sequence around the boundary between exon 2 and intron 2 shown in SEQ ID NO: 41. The heterozygosity is evidenced by the sequence read at position 6 of SEQ ID NO: 41, which shows both G and A peaks and is therefore represented by N.

(B) illustrates RT-PCR amplification of cDNA from subjects from (A) across exons 2-3 of the plekhm1 gene using primers 2A (SEQ ID NO: 36) and 3B (SEQ ID NO: 37) and indicates the sequence at the exon-intron or exon-exon junctions of the corresponding RT-PCR products; M labels Mw marker, 1 corresponds to father, 2 to mother and 3 to affected daughter from the pedigree in (A); C is unrelated control; the shown nucleotide sequences of the highest band correspond to positions 277-307 and 550-577 of SEQ ID NO: 10 (mutant PLEKHM1 cDNA with insertion of 262 nucleotides from intron 2) and the corresponding portion of the mutant truncated human PLEKHM1 protein (positions 93-99 of SEQ ID NO: 11); the shown nucleotide sequences of the middle band correspond to positions 277-307 and 383-410 of SEQ ID NO: 9 (mutant PLEKHM1 cDNA with insertion of 95 nucleotides from intron 2) and the corresponding portion of the mutant truncated human PLEKHM1 protein (positions 93-99 of SEQ ID NO: 11); the nucleotide sequences of the highest band correspond to positions 277-315 of SEQ ID NO: 7 (wild-type PLEKHM1 cDNA) and the corresponding portion of the wild-type human PLEKHM1 protein (positions 93-105 of SEQ ID NO: 8)

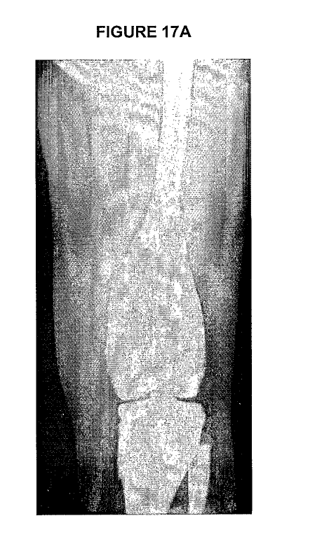

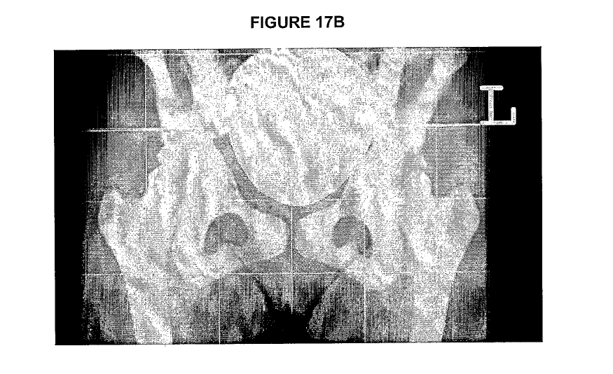

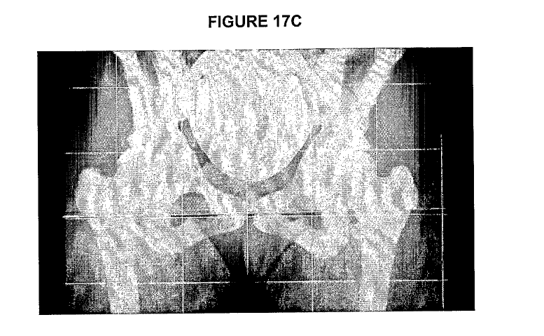

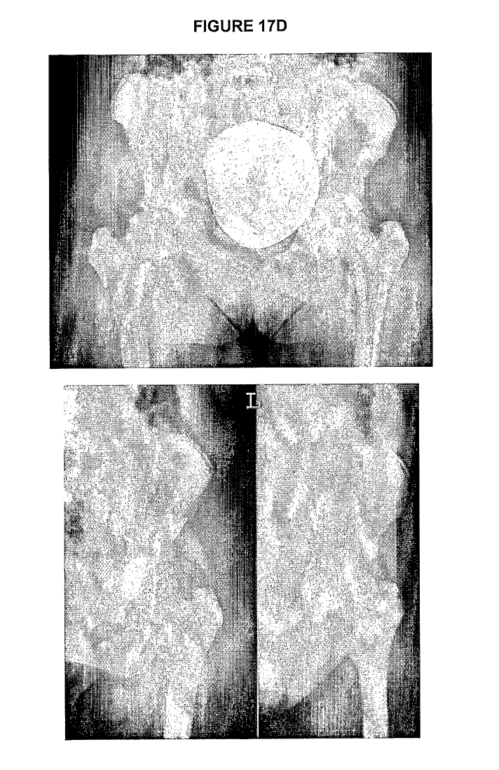

FIG. 17A illustrates “Erlenmeyer flask” deformity of the distal femora in a patient homozygous for a G to A transition at position +1 of the donor splice site of intron 2 of the PLEKHM1 gene (IVS2+1 G to A). The patient was examined in 1997.

FIG. 17B illustrates a follow-up radiograph of the same patient in June 2003, when there were no complaints of pain, showing normal joint space in the left hip.

FIG. 17C illustrates a follow-up radiograph of the same patient in January 2004, when there were no complaints of pain, showing the narrowing of the joint space of the left hip.

FIG. 17D illustrates a follow-up radiograph of the same patient in October 2004, when there were complaints of severe pain in the left hip, showing the narrowing of the joint space of the left hip.

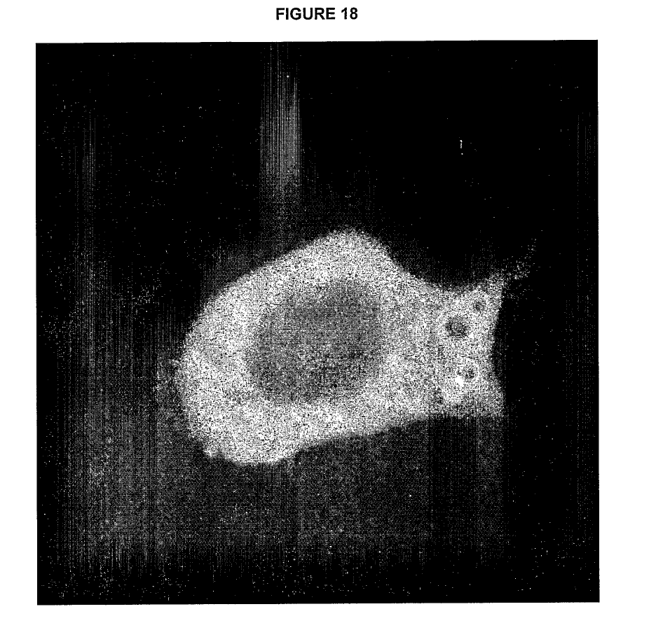

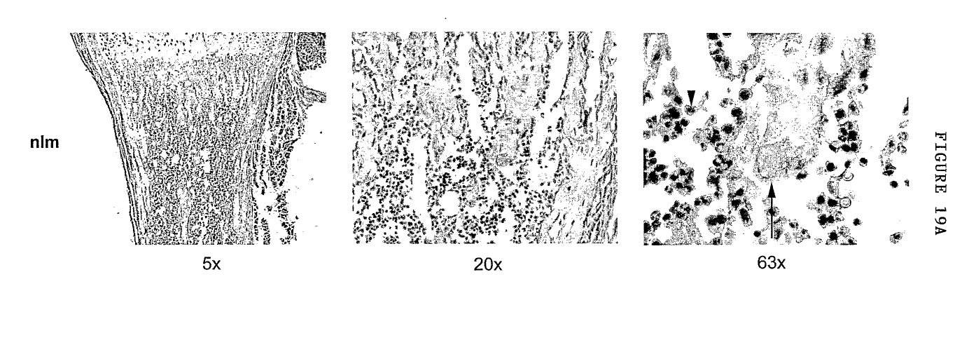

FIG. 18 illustrates detection of EGFP-PLEKHM1 fusion protein expressed in transfected HEK cells using confocal microscopy; a diffuse cytoplasmic distribution is observed along with the appearance of abnormally large vesicles.

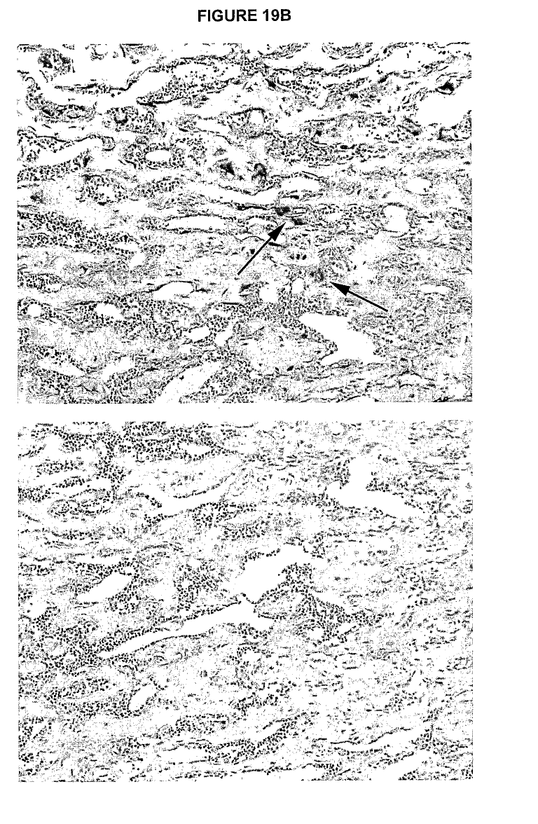

FIG. 19A illustrates immuno-histochemistry on bone slides of wild type rats using the anti-plekhm1B antibody (arrows). Counterstaining with hematoxyline-eosin is performed to detect nuclei (arrowheads). The slides were viewed with conventional microscopy at 5× (A), 20× (B) and 63× (C) magnification. The plekhm1 protein is primarily expressed in osteoclasts (arrows).

FIG. 19B illustrates immuno-histochemistry on bone slides of wild-type rats using the anti-plekhm1A antibody (arrows) without (top) and with (bottom) the addition of the corresponding peptide. Counterstaining with hematoxylin-eosin is performed to detect nuclei. The slides were viewed with conventional microscopy at 10× magnification. Staining was present in the absence of the corresponding peptides (top, arrows). Adding the corresponding peptides to the anti-plekhm1A antibody almost completely neutralized the staining.

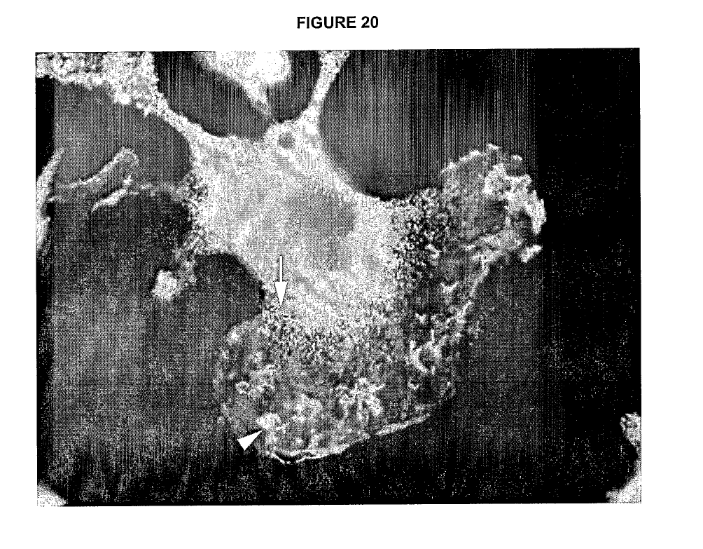

FIG. 20 illustrates detection of the plekhm1 protein in a multinuclear osteoclast by fluorescence microscopy. Two nuclei were observed in this cell and the plekhm1 protein staining suggested both vesicular (arrow) and cytoskeletal (arrowhead) localization.

FIG. 21 represents the nucleic acid sequence of the mutant human PLEKHM1 cDNA resulting from the IVS2+1 G to A mutation (indicated in bold) having an insertion of 95 nucleotides from intron 2 (underlined) (SEQ ID NO: 9).

FIG. 22 represents the nucleic acid sequence of the mutant human PLEKHM1 cDNA resulting from the IVS2+1 G to A mutation (indicated in bold) having an insertion of 262 nucleotides from intron 2 (underlined) (SEQ ID NO: 10).

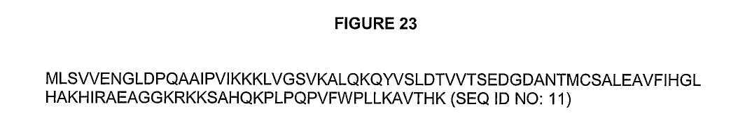

FIG. 23 represents the amino acid sequence of the truncated human PLEKHM1 polypeptide (SEQ ID NO: 11) encoded by the cDNA sequences shown in FIGS. 21 and 22 which result from the IVS2+1 G to A mutation.

### DETAILED DESCRIPTION OF THE INVENTION

The present invention is directed to the use of nucleic acid sequences encoding a mammalian PLEKHM1 polypeptide in diagnostic and therapeutic methods for diagnosing and/or preventing and/or treating bone-related disorders resulting from a disturbance in bone homeostasis. The present invention is directed to the use of non-mutated and/or mutated PLEKHM1 gene sequences from different mammalian organisms, including human; rat or mouse, in diagnostic and therapeutic methods for diagnosing and/or preventing and/or treating bone-related disorders.

### Identified Nucleic Acids, Characterization Thereof

The invention provides the sequence identities corresponding to nucleic acids (SEQ ID NO: 1, 3, 5, and 7 and SEQ ID NO: 9 and 10) and corresponding polypeptides (SEQ ID NO: 2, 4, 6 and 8 and SEQ ID NO: 11). SEQ ID NO: 1, 5 or 7 relate to the PLEKHM1 gene, respectively in rat, mouse and human. SEQ ID NO: 2, 6 or 8 relate to the polypeptide sequence, encoded by a PLEKHM1 gene in respectively rat, mouse and human. SEQ ID NO: 3 relate to a mutated rat PLEKHM1 gene. SEQ ID NO: 4 relates to the polypeptide sequence encoded by a mutated PLEKHM1 gene in rat. SEQ IDs NO: 9 and 10 relate to alternative cDNA sequences resulting from a mutation (IVS2+1 G to A mutation) in human PLEKHM1 gene. SEQ ID NO: 11 relates to the polypeptide sequence encoded by these alternative cDNA sequences (transcripts).

The invention further provides sequence identities corresponding to nucleic acids SEQ ID NO: 12-41. These represent sequences of primers used in the present invention or partial sequence reads obtained by the present invention.

The rat PLEKHM1 cDNA comprises 3180 bp and is represented in FIG. 1. The corresponding polypeptide sequence thereof consists of 1059 amino acids and is represented in FIG. 2. The mouse PLEKHM1 cDNA comprises 3225 bp and is represented in FIG. 5. The corresponding polypeptide sequence thereof consists of 1074 amino acids and is represented in FIG. 6. The human PLEKHM1 cDNA comprises 3171 bp and is represented in FIG. 7. The corresponding polypeptide sequence thereof consists of 1056 amino acids and is represented in FIG. 8. Mutated PLEKHM1 DNA was isolated from an ia rat as described in example 1. The mutated PLEKHM1 cDNA comprises 3179 bp and is represented in FIG. 3. The corresponding mutated polypeptide sequence thereof consists of 342 amino acids and is represented in FIG. 4. Mutated human PLEKHM1 DNA and cDNAs were isolated from a human osteopetrosis patient as described in examples 6 and 7. The two alternative mutated PLEKHM1 cDNAs comprise 3266 bp and 3433 bp and are represented in FIGS. 21 and 22, respectively. The corresponding mutated polypeptide sequence thereof consists of 99 amino acids and is represented in FIG. 23.

In a first embodiment, the present invention provides a nucleic acid comprising a DNA sequence as given in SEQ ID NO: 1, 3, 5 or 7 or SEQ ID NO: 9 or 10, or the complement thereof.

In another embodiment, the present invention provides a nucleic acid comprising a DNA sequence as given in SEQ ID NO: 12-41, or the complement thereof.

The present invention also relates to a nucleic acid comprising the RNA sequence corresponding to SEQ ID NO: 1, 3, 5 or 7 or SEQ ID NO: 9 or 10, or a complement thereof.

The present invention also relates to a nucleic acid comprising the RNA sequence corresponding to SEQ ID NO: 12-41, or a complement thereof.

In yet another embodiment the invention relates to a nucleic acid specifically hybridizing to the nucleotide sequence as defined in SEQ ID NO: 1, 3, 5, or 7 or SEQ ID NO: 9 or 10, or the complement thereof. In an example, such nucleic acids may comprise DNA or RNA sequence corresponding to SEQ ID NO: 12-41, or a complement thereof.

Another embodiment the invention relates to a nucleic acid having a nucleotide sequence which is at least 65%, and preferably at least 75%, and preferably at least 85% and preferably at least 95% and preferably at least 99% identical to the sequence as defined in SEQ ID NO: 1, 3, 5, or 7 or SEQ ID NO: 9 or 10, or the complement thereof.

The present invention is also directed to variants of the nucleotide sequence of the nucleic acid disclosed in SEQ ID NO: 1, 3, 5, or 7 or SEQ ID NO: 9 or 10, or the corresponding complementary strand.

The present invention is also directed to variants of the nucleotide sequence of the nucleic acid disclosed in SEQ ID NO: 12-41 or the corresponding complementary strand.

The present invention is also directed to nucleic acid molecules which comprise, or alternatively consist of, a nucleotide sequence which is at least 65%, 70%, 75%, 80%, 85%, 90%, 95%, 96%, 97%, 97.5%, 98%, 98.5%, 99% or 99.5% identical to the nucleotide sequences as represented in SEQ ID NO: 1, 3, 5 or 7 or SEQ ID NO: 9 or 10, or the corresponding complementary strand, or parts thereof. Said parts are preferably unique parts.

The present invention is also directed to nucleic acid molecules which comprise, or alternatively consist of, a nucleotide sequence which is at least 65%, 70%, 75%, 80%, 85%, 90%, 95%, 96%, 97%, 97.5%, 98%, 98.5%, 99% or 99.5% identical to the nucleotide sequences as represented in SEQ ID NO: 12-41 or the corresponding complementary strand, or parts thereof. Said parts are preferably unique parts.

By a nucleic acid having a nucleotide sequence at least, for example, 95% “identical” to a reference nucleotide sequence of the present invention, it is intended that the nucleotide sequence of said nucleic acid is identical to the reference sequence except that the nucleotide sequence may include up to five point mutations per each 100 nucleotides of the reference nucleotide sequence. In other words, to obtain a nucleic acid having a nucleotide sequence of at least 95% identity to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. As a practical matter, whether any particular nucleic acid molecule is at least 65%, 70%, 75%, 80%, 85%, 90%, 95%, 96%, 97%, 97.5%, 98%, 98.5%, 99% or 99.5% identical to a nucleotide sequence of the present invention can be determined using known algorithms. A preferred method for determining the best overall match between a query sequence (a sequence of the present invention) and a subject sequence can be determined using a Blast search.

In another embodiment the invention relates to a nucleic acid encoding a protein with an amino acid sequence which is at least 65%, 70%, 75%, 80%, 85%, 90%, 95%, 96%, 97%, 97.5%, 98%, 98.5%, 99% or 99.5% identical to the nucleotide identical to the amino acid sequence as given in SEQ ID NO: 2, 4, 6 or 8 or SEQ ID NO: 11.

In yet another embodiment the invention relates to a nucleic acid which is degenerated as a result of the genetic code to a nucleotide sequence of a nucleic acid as given in SEQ ID NO: 1, 3, 5 or 7 or SEQ ID NO: 9 or 10, or as defined above.

Another embodiment the invention relates to a nucleic acid which is diverged due to differences in codon usage between organisms to a nucleotide sequence encoding a protein as given in SEQ ID NO: 2, 4, 6 or 8, or SEQ ID NO: 11, or as defined above.

The invention also relates to a nucleic acid which is diverged due to the differences between alleles encoding a protein as given in SEQ ID NO: 2, 4, 6 or 8 or SEQ ID NO: 11, or as defined above.

In another embodiment the invention relates to a nucleic acid encoding an immunologically active and/or functional fragment of a protein encoded by a DNA sequence as given in SEQ ID NO: 1, 3, 5 or 7 or SEQ ID NO: 9 or 10.

In another embodiment the invention relates to a nucleic acid encoding an immunologically active and/or functional fragment of a protein encoded by a DNA sequence as given in SEQ ID NO: 1, 5 or 7.

With “immunologically active” is meant that a molecule or specific fragments thereof such as epitopes or haptens are recognized by, i.e. bind by antibodies.

The term “functional fragments” or “functional parts” refers to any part of the nucleotide sequence of the present invention, which exhibits substantially a similar, but not necessarily identical, activity as the complete nucleotide sequence.

Also part of the invention comprises a nucleic acid encoding a gene family member of the nucleic acid as given in SEQ ID NO: 1, 3, 5 or 7.

Furthermore, the invention relates to a nucleic acid encoding a protein as defined in SEQ ID NO: 2, 4, 6 or 8 or SEQ ID NO: 11, or a nucleic acid as defined in above, wherein said sequence is DNA, cDNA, genomic DNA or synthetic DNA.

Furthermore, the invention relates to a nucleic acid encoding a protein as defined in SEQ ID NO: 2, 6 or 8, or a nucleic acid as defined in above, wherein said sequence is DNA, cDNA, genomic DNA or synthetic DNA.

In a preferred embodiment, the invention provides a nucleic acid molecule of at least 10 nucleotides in length specifically hybridising with any of the nucleic acids according to the present invention. In particular, longer nucleic acid molecules are contemplated, i.e. of about 15, 20, 25, 30, 40, 50, 75, 100, 200 or even more nucleotides. It is to be understood that also shorter probes may be useful (having for instance 10, 11, 12, 13 or 14 nucleotides). Different types of hybridisation techniques and formats are well known in the art. The said nucleic acid molecule may be labelled with, for example, a radioactive isotope or an immunofluorescent compound, thereby allowing the detection of the hybrid. As such, the present invention provides methods for detecting the nucleic acids according to the present invention.

In a further embodiment, the invention provides a nucleic acid molecule of at least 15 nucleotides in length as described above, wherein said nucleic acid molecule is liable to act as a primer for specifically amplifying a nucleic acid of the present invention, or a part thereof. It is to be understood that said primers can be shorter, e.g. 10, 11, 12, 13, or 14 nucleotides, or longer, e.g. 16, 17, 18, 19, 20, 25, or 30 nucleotides.

Sets of said primers may be used in any well described amplification technique known in the art such as Polymerase Chain Reaction (PCR), TMA or NASBA techniques, thereby allowing the amplification and subsequent detection of the nucleic acid of the present invention. Preferably, said primers may also be used to specifically amplify the nucleic acids of the present invention. As such, the present invention provides methods for detecting the nucleic acids of the present invention. Exemplary primers and primer sets are listed in Table 2 and SEQ ID NO: 14-40.

Nucleic acids which specifically hybridize to any of the strands of the nucleic acid molecules of the present invention as specified under SEQ ID NO: 1, 3, 5 or 7 or SEQ ID NO: 9 or 10 under stringent hybridization conditions or lower stringency conditions are also particularly encompassed by the present invention. “Stringent hybridisation conditions” refers to an overnight incubation at 68° C. in a solution comprising 5×SSC (750 mM NaCl, 75 mM trisodium citrate), 50 mM sodium phosphate (pH 7.6), 5× Denhardt's solution, 10% dextran sulfate and 20 μg/ml denatured sheared salmon sperm DNA, followed by washing the filters in 0.1×SSC at about 65° C. Changes in the stringency of hybridisation are primarily accomplished through the manipulation of the SSC dilution in the washing steps (higher concentration SSC in washing buffer results in lower stringency) and the temperature (lower washing temperature results in lower stringency). For example, lower stringency conditions include washes performed at 1×SSC and at 55-60° C. Hybridisation under high and low stringency conditions are principles which are well understood by the person skilled in the art (see, for instance, Sambrook et al. Molecular Cloning: A laboratory manual. Cold Spring Harbor laboratory press 1989).

Methods which are well known to those skilled in the art may be used to construct expression vectors containing at least a fragment of the nucleic acids of the present invention together with appropriate transcriptional and translational control elements. These methods include in vitro recombinant DNA techniques, synthetic techniques, and in vivo genetic recombination. Such techniques are described, for example, in Sambrook et al. (1989).

Methods which are well known to those skilled in the art may be used to construct expression vectors containing at least a fragment of the nucleic acids of the present invention together with appropriate transcriptional and translational control elements. These methods include in vitro recombinant DNA techniques, synthetic techniques, and in vivo genetic recombination. Such techniques are described, for example, in Sambrook et al. (1989).

The present invention relates also to vectors comprising the nucleic acid of the present invention. The present invention particularly contemplates recombinant expression vectors, said vectors comprising a vector sequence, an appropriate prokaryotic, eukaryotic or viral or synthetic promoter sequence followed by the sample nucleic acid of the present invention. Preferably, the vector used for expressing the sample nucleic acid according to the present invention can be a vector for expression in E. coli, a yeast shuttle vector, or a yeast two-hybrid vector, a plant vector, an insect vector, a mammalian expression vector, including but not limited to, a herpes virus vector, a baculovirus vector, a lentivirus vector, a retrovirus vector, an alphavirus vector, an adenoviral vector or any combination thereof.

In a preferred embodiment, the invention provides a vector comprising a nucleic acid sequence of the present invention. Preferably, said nucleic acid represented by SEQ ID NO: 1, 3, 5 or 7 or SEQ ID NO: 9 or 10, or variants, fragments or homologues thereof. Preferably, said nucleic acid represented by SEQ ID NO: 1, 5 or 7 or variants, fragments or homologues thereof.

In a preferred embodiment said vector is an expression vector wherein the nucleotide sequence is operably linked to one or more control sequences allowing the expression of said sequence in prokaryotic and/or eukaryotic host cells.

In another preferred embodiment said vector is an adenoviral vector.

The nucleotide sequences of the present invention can be engineered using methods generally known in the art in order to alter protein encoding sequences for a variety of reasons, including but not limited to, alterations which modify the cloning, processing, and/or expression of the gene product. DNA shuffling by random fragmentation or PCR reassembly of gene fragments or synthetic oligonucleotides may be used to engineer the nucleotide sequences. For example, site-directed mutagenesis may be used to insert new restriction sites, alter glycosylation patterns, change codon preference, produce splice variants, introduce mutations, and so forth.

Furthermore, natural, modified, or recombinant nucleotide sequences may be ligated to partial or complete nucleic acid sequences of the present invention to encode a fusion protein. For example, to screen peptide libraries for inhibitors of the product of the nucleic acids of the present invention, it may be useful to encode a chimeric protein that can be recognised by a commercially available antibody. A fusion protein may also be engineered to contain a cleavage site located between the protein coding sequence and the heterologous protein sequence, so that the protein may be cleaved and purified away from the heterologous moiety.

In a further embodiment, the invention provides a host cell containing an integrated or episomal copy of any of the nucleotide sequences of the present invention or any functional parts thereof. In a more preferred embodiment, the invention provides a host cell containing a vector comprising a nucleic acid sequence according to the present invention.

The latter host cell can be obtained from any organism including, but not limited to, mammals, such as humans, canines and rodents, amphibia, reptiles, birds, fish, nematodes, yeast, fungi, bacteria, insects and plants.

In this regard, the term “functional parts” refers to any part of the nucleotide sequence of the present invention which exhibits substantially a similar, but not necessarily identical, activity as the complete nucleotide sequence.

In a preferred embodiment, the invention provides an isolated polypeptide encodable by any of the herein mentioned nucleic acids, or a variant or a derivative thereof, or an immunologically active and/or functional fragment thereof.

It is thus understood that the present invention relates to a polypeptide having an amino acid sequence as given in SEQ ID NO: 2, 4, 6 or 8 or SEQ ID NO: 11, or a variant or a derivative thereof, or an immunologically active and/or functional fragment thereof.

It is also understood that the present invention relates to a polypeptide having an amino acid sequence as given in SEQ ID NO: 2, 6 or 8, or a variant or a derivative thereof, or an immunologically active and/or functional fragment thereof.

“Variants” of a protein of the invention are those peptides, oligopeptides, polypeptides, proteins and enzymes which contain amino acid substitutions, deletions and/or additions relative to the said protein with respect to which they are a homologue, while maintaining the function of the protein. In other words, the term “variant” refers to a polypeptide or protein differing from the polypeptide or protein of the present invention, but retaining essential properties thereof.

In the present invention, the functional activity of a protein relates to the “function”, which refers to the ability per se to modulate bone formation/bone resorption and to the “activity” which refers to the amount of this ability (a quantitative measure). Generally, variants are overall closely similar, and, in many regions, identical to the polypeptide or protein of the present invention. For example, a homologue of said protein will consist of a bio-active amino acid sequence variant of said protein. To produce such homologues, amino acids present in the said protein can be replaced by other amino acids having similar properties, for example hydrophobicity, hydrophilicity, hydrophobic moment, antigenicity, propensity to form or break α-helical structures or β-sheet structures, and so on. Amino acid substitutions are typically of single residues, but may be clustered depending upon functional constraints placed upon the polypeptide; insertions will usually be of the order of about 1-10 amino acid residues and deletions will range from about 1-20 residues. Preferably, amino acid substitutions will comprise conservative amino acid substitutions.

Amino acid variants of a protein of the invention may readily be made using peptide synthetic techniques well known in the art, such as solid phase peptide synthesis and the like, or by recombinant DNA manipulations. The manipulation of DNA sequences to produce variant proteins which manifest as substitution, insertion or deletion variants are well known in the art.

“Derivatives” of a protein of the invention are those peptides, oligopeptides, polypeptides, proteins and enzymes which comprise at least about 5 contiguous amino acid residues of said polypeptide but which retain the biological activity of said protein. Preferably said derivatives will comprise at least 6, 7, 8, 9, 10, 11, 12, 13, 14, or 15 contiguous amino acid residues of said protein. A “derivative” may further comprise additional naturally-occurring, altered glycosylated, acylated or non-naturally occurring amino acid residues compared to the amino acid sequence of a naturally-occurring form of said polypeptide. Alternatively or in addition, a derivative may comprise one or more non-amino acid substituents compared to the amino acid sequence of a naturally-occurring form of said polypeptide, for example a reporter molecule or other ligand, covalently or non-covalently bound to the amino acid sequence such as, for example, a reporter molecule to facilitate its detection.

In the context of the current invention are embodied homologues, derivatives and/or immunologically active fragments of any of the newly identified sequences as defined above.

With “immunologically active” is meant that a molecule or specific fragments thereof such as epitopes or haptens are recognized by, i.e. bind by antibodies.

The term “homologue” relates to the molecule in a non-human species, that corresponds to the molecule of the present invention.

The term “functional fragments” or “functional parts” refers to any part of the nucleotide sequence of the present invention, which exhibits substantially a similar, but not necessarily identical, activity as the complete nucleotide sequence.

In a preferred embodiment, the invention provides a method for producing the polypeptide of the present invention, the method comprising culturing host cells comprising a nucleic acid of the invention as defined above under conditions allowing the expression of the polypeptide and recovering the produced polypeptide from the culture. Alternative methods for producing said polypeptides of the invention are well known in the art, such as, for example, chemical synthesis.

The present invention is also directed to polypeptides, which comprise, or alternatively consist of, an amino acid sequence which is at least 65%, 70%, 75%, 80%, 85%, 90%, 95%, 96%, 97%, 97.5%, 98%, 98.5%, 99% or 99.5% identical to the amino acid sequences of the present invention, wherein said amino acid sequence of the invention, the so-called reference sequence, is at least 30 amino acids in length. However, for reference sequences smaller than 30 amino acids the polypeptide must consist of an amino acid sequence which is at least 65%, 70%, 75%, 80%, 85%, 90%, 95%, 96%, 97%, 97.5%, 98%, 98.5%, 99% or 99.5% identical to the reference sequence.

By a polypeptide having an amino acid sequence of at least, for example, 95% “identity” to a reference amino acid sequence of the present invention, it is intended that the amino acid sequence of the polypeptide is identical to the reference sequence except that the amino acid sequence may include up to five amino acid alterations per each 100 amino acids of the reference polypeptide amino acid sequence. In other words, to obtain a polypeptide having an amino acid sequence at least 95% identical to a reference amino acid sequence, up to 5% of the amino acids in the reference sequence may be deleted or substituted with another amino acid, or a number of amino acids up to 5% of the total amino acids in the reference sequence may be inserted into the reference sequence. As a practical matter, whether any particular polypeptide is at least 65%, 70%, 75%, 80%, 85%, 90%, 95%, 96%, 97%, 97.5%, 98%, 98.5%, 99% or 99.5% identical to a polypeptide sequence of the present invention can be determined using known algorithms. A preferred method for determining the best overall match between a query sequence (a sequence of the present invention) and a subject sequence can be determined using BLASTp (Altschul et al., 1997).

### Applications of Identified Nucleic Acids

The present invention relates to the use of a nucleic acid according to the present invention for regulating bone homeostasis. As indicated above, the term “regulating bone homeostasis” as used herein refers to the activity of maintaining a suitable balance between bone formation and bone resorption. Disruption of this balance may induce some serous bone-related diseases such as, but not limited to osteoporosis, osteopetroses, etc. . . . The term encompasses, e.g., decreasing bone resorption in order to increase bone mass or alternatively, increasing bone resorption in order to decrease bone mass.

In a preferred embodiment, the invention relates to the use of a nucleic acid comprising a DNA sequence as given in SEQ ID NO: 1, 3, 5 or 7 or SEQ ID NO: 9 or 10, or the complement thereof, or to a polypeptide having an amino acid sequence as given in SEQ ID NO: 2, 4, 6 or 8 or SEQ ID NO: 11, or a variant, derivative thereof, or an immunologically active and/or functional fragment, for regulating bone homeostasis.

In a preferred embodiment, the invention relates to the use of a nucleic acid comprising a DNA sequence as given in SEQ ID NO: 1, 5, or 7 or the complement thereof, or to a polypeptide having an amino acid sequence as given in SEQ ID NO: 2, 6 or 8, or a variant, derivative thereof, or an immunologically active and/or functional fragment, for regulating bone homeostasis.

The present invention thus also relates to the use of a nucleic acid comprising a nucleic acid sequence which is at least 65%, 70%, 75%, 80%, 85%, 90%, 95%, 96%, 97%, 97.5%, 98%, 98.5%, 99% or 99.5% identical to SEQ ID NO: 1, 3, 5 or 7, or SEQ ID NO: 9 or 10, or a functional fragment thereof, for regulating bone homeostasis.

The present invention thus also relates to the use of a nucleic acid comprising a nucleic acid sequence which is at least 65%, 70%, 75%, 80%, 85%, 90%, 95%, 96%, 97%, 97.5%, 98%, 98.5%, 99% or 99.5% identical to SEQ ID NO: 1, 5 or 7, or a functional fragment thereof, for regulating bone homeostasis.

Also, the present invention relates to the use of a protein comprising an amino acid sequence which is at least 65%, 70%, 75%, 80%, 85%, 90%, 95%, 96%, 97%, 97.5%, 98%, 98.5%, 99% or 99.5% identical to SEQ ID NO: 2, 4, 6 or 8 or SEQ ID NO: 11, or a functional fragment thereof, for regulating bone homeostasis.

Also, the present invention relates to the use of a protein comprising an amino acid sequence which is at least 65%, 70%, 75%, 80%, 85%, 90%, 95%, 96%, 97%, 97.5%, 98%, 98.5%, 99% or 99.5% identical to SEQ ID NO: 2, 6 or 8, or a functional fragment thereof, for regulating bone homeostasis.

### Antibodies

In a preferred embodiment, the invention provides an antibody specifically recognizing the polypeptides of the present invention, or a specific epitope of said polypeptide. The term “epitope” refers to portions of a polypeptide having antigenic or immunogenic activity in an animal, preferably a mammal, and most preferably in a human. Epitope-bearing polypeptides of the present invention may be used to induce antibodies according to methods well known in the art including, but not limited to, in vivo immunisation, in vitro immunisation, phage display methods or ribosome display.

The antibody of the present invention relate to any polyclonal or monoclonal antibody binding to a protein of the present invention. The term “monoclonal antibody” used herein refers to an antibody composition having a homogeneous antibody population. The term is not limiting regarding the species or source of the antibody, nor is it intended to be limited by the manner in which it is made. Hence, the term “antibody” contemplates also antibodies derived from e.g., camels, or the genus lama. Thus, the term “antibody” also refers to antibodies derived from phage display technology or drug screening programs. In addition, the term “antibody” also refers to humanised antibodies in which at least a portion of the framework regions of an immunoglobulin are derived from human immunoglobulin sequences and single chain antibodies as described in U.S. Pat. No. 4,946,778 and to fragments of antibodies such as F_{ab}, F_{(ab)2}, Fᵥ, and other fragments which retain the antigen binding function and specificity of the parent antibody. The term “antibody” also refers to diabodies, triabodies or multimeric (mono-, bi -, tetra- or polyvalent/mono-, bi- or polyspecific) antibodies, as well as enzybodies, i.e. artificial antibodies with enzyme activity. Combinations of antibodies with any other molecule that increases affinity or specificity, are also contemplated within the term “antibody”. Antibodies also include modified forms (e.g. mPEGylated or polysialylated form (Fernandes & Gregoriadis, 1997; Acta 1341:26-34) as well as covalently or non-covalently polymer bound forms. In addition, the term “antibody” also pertains to antibody-mimicking compounds of any nature, such as, for example, derived from lipids, carbohydrates, nucleic acids or analogues e.g. PNA, aptamers.

In specific embodiments, antibodies of the present invention cross-react with murine, goat, rat and/or rabbit homologues of human proteins and the corresponding epitopes thereof. Further included in the present invention are antibodies that bind polypeptides encoded by nucleic acids that hybridise to a polynucleotide of the present invention under stringent hybridisation conditions (as described herein).

As such, the present invention provides a method for detecting the polypeptides of the present invention, the method comprising the use of the antibodies in immunoassays for qualitatively or quantitatively measuring levels of the polypeptides of the present invention in biological samples.

Thus the invention contemplates also a method for detecting a nucleic acid or a polypeptide as described herein, preferably by an antibody of the present invention.

Antibodies of the present invention may act as agonists or antagonists of the polypeptides of the present invention. Preferably, antibodies of the present invention bind an antigenic epitope as disclosed herein, or a particular portion of the proteins of the present invention.

Antibodies of the present invention may be used, for example, but not limited to, to purify, detect, target, and/or inhibit the activity of the polypeptides of the present invention including both in vitro and in vivo diagnostic and therapeutic methods, as well as in drug screens (see infra). In particular, the antibodies of the present invention may be used to regulate bone homeostasis. In a preferred embodiment, the invention relates to the use of an antibody specifically recognizing a polypeptide encodable by a nucleic acid according to the present invention, or a specific epitope of said polypeptide for regulating bone homeostasis.

### Therapy

As mentioned above, since the PLEKHM1 gene and its corresponding protein according to the invention play a basic role in bone homeostasis, they are particularly suitable for use in therapeutic approaches relating to bone disorders. Bone disorders or bone-related disorders denote diseases involving a disregulated bone homeostasis, as detailed elsewhere in this specification.

The present invention therefore relates to a nucleic acid, a polypeptide or antibody according to the present invention, for use as a medicament. These nucleic acids, polypeptides and/or antibodies are suitable for regulating bone homeostasis, which makes them particularly useful for therapeutic approaches relating to bone-related disorders, in particular diseases involving a disregulated bone homeostasis.

In a preferred embodiment, the invention relates to the use of a nucleic acid comprising a DNA sequence as given in SEQ ID NO: 1, 3, 5 or 7 or SEQ ID NO: 9 or 10, or the complement thereof, or to a polypeptide having an amino acid sequence as given in SEQ ID NO: 2, 4, 6 or 8 or SEQ ID NO: 11, or a variant, derivative thereof, or an immunologically active and/or functional fragment, as a medicament.

In a preferred embodiment, the invention relates to the use of a nucleic acid comprising a DNA sequence as given in SEQ ID NO: 1, 5 or 7, or the complement thereof, or to a polypeptide having an amino acid sequence as given in SEQ ID NO: 2, 6 or 8, or a variant, derivative thereof, or an immunologically active and/or functional fragment, as a medicament.

In a preferred embodiment, the invention relates to the nucleic acid comprising a DNA sequence as given in SEQ ID NO: 1, 3, 5 or 7 or SEQ ID NO: 9 or 10, or the complement thereof, or to a polypeptide having an amino acid sequence as given in SEQ ID NO: 2, 4, 6 or 8 or SEQ ID NO: 11, or a variant, derivative thereof, or an immunologically active and/or functional fragment, for use as a medicament. In a preferred embodiment, the invention relates to the nucleic acid comprising a DNA sequence as given in SEQ ID NO: 1, 5 or 7, or the complement thereof, or to a polypeptide having an amino acid sequence as given in SEQ ID NO: 2, 6 or 8, or a variant, derivative thereof, or an immunologically active and/or functional fragment, for use as a medicament.

In another embodiment, the invention relates to the use of a nucleic acid according to the present invention, or a polypeptide according to the present invention, or an antibody according to the present invention for the preparation of a medicament for preventing, treating and/or alleviating diseases involving a disregulated bone homeostasis.

In an embodiment, the invention relates to a method for preventing, treating and/or alleviating a bone-related disorder, in particular a disease involving a disregulated bone homeostasis, comprising administering to an individual in need of such treatment a therapeutically effective amount of a nucleic acid according to the present invention, or of a polypeptide according to the present invention, or of an antibody according to the present invention

Accordingly, in an embodiment, the invention relates to the use of a nucleic acid encoding a mammalian PLEKHM1 polypeptide or a variant or a derivative thereof or an immunologically active and/or functional fragment thereof, or of the complement of said nucleic acid for the preparation of a medicament for preventing, treating and/or alleviating a bone related disorder, in particular a disease involving a disregulated bone homeostasis.

In an embodiment, the invention relates to a method for preventing, treating and/or alleviating a bone related disorder, in particular a disease involving a disregulated bone homeostasis, comprising administering to an individual in need of such treatment a therapeutically effective amount of a nucleic acid encoding a mammalian PLEKHM1 polypeptide or a variant or a derivative thereof or an immunologically active and/or functional fragment thereof, or of the complement of said nucleic acid.

In an embodiment, the invention relates to the use of a nucleic acid encoding a mammalian PLEKHM1 polypeptide or a variant or a derivative thereof or an immunologically active and/or functional fragment thereof, wherein said nucleic acid comprises a mutation in its nucleotide sequence, or of the complement of said nucleic acid for the preparation of a medicament for preventing, treating and/or alleviating a bone related disorder, in particular a disease involving a disregulated bone homeostasis.

In an embodiment, the invention relates to a method for preventing, treating and/or alleviating a bone related disorder, in particular a disease involving a disregulated bone homeostasis, comprising administering to an individual in need of such treatment a therapeutically effective amount of a nucleic acid encoding a mammalian PLEKHM1 polypeptide or a variant or a derivative thereof or an immunologically active and/or functional fragment thereof, wherein said nucleic acid comprises a mutation in its nucleotide sequence, or of the complement of said nucleic acid.

In a preferred embodiment, the invention relates to the use of a nucleic acid comprising a DNA sequence as given in SEQ ID NO: 1, 3, 5 or 7 or SEQ ID NO: 9 or 10, or the complement thereof, or to a polypeptide having an amino acid sequence as given in SEQ ID NO: 2, 4, 6 or 8 or SEQ ID NO: 11, or a variant, derivative thereof, or an immunologically active and/or functional fragment, for the preparation of a medicament for preventing, treating and/or alleviating diseases involving a disregulated bone homeostasis.

In a preferred embodiment, the invention relates to the use of a nucleic acid comprising a DNA sequence as given in SEQ ID NO: 1, 5 or 7, or the complement thereof, or to a polypeptide having an amino acid sequence as given in SEQ ID NO: 2, 6 or 8, or a variant, derivative thereof, or an immunologically active and/or functional fragment, for the preparation of a medicament for preventing, treating and/or alleviating diseases involving a disregulated bone homeostasis.

In an embodiment, the invention relates to the use of a nucleic acid comprising a DNA sequence as given in SEQ ID NO: 1, 3, 5 or 7 or SEQ ID NO: 9 or 10, or the complement thereof for the preparation of a medicament for preventing, treating and/or alleviating a bone related disorder, in particular a disease involving a disregulated bone homeostasis.

In an embodiment, the invention relates to the use of a nucleic acid comprising a DNA sequence as given in SEQ ID NO: 1, 5 or 7, or the complement thereof for the preparation of a medicament for preventing, treating and/or alleviating a bone related disorder, in particular a disease involving a disregulated bone homeostasis.

In an embodiment, the invention relates to a method for preventing, treating and/or alleviating a bone related disorder, in particular a disease involving a disregulated bone homeostasis, comprising administering to an individual in need of such treatment a therapeutically effective amount of a nucleic acid comprising a DNA sequence as given in SEQ ID NO: 1, 3, 5 or 7 or SEQ ID NO: 9 or 10, or of the complement thereof.

In an embodiment, the invention relates to a method for preventing, treating and/or alleviating a bone related disorder, in particular a disease involving a disregulated bone homeostasis, comprising administering to an individual in need of such treatment a therapeutically effective amount of a nucleic acid comprising a DNA sequence as given in SEQ ID NO: 1, 5 or 7, or of the complement thereof.

In an embodiment, the invention relates to the use of a nucleic acid comprising a nucleic acid sequence which is at least 65%, preferably at least 75%, preferably at least 85% and preferably at least 95% and preferably at least 99% identical to SEQ ID NO: 1, 3, 5 or 7 or SEQ ID NO: 9 or 10, or the complement thereof, for the preparation of a medicament for preventing, treating and/or alleviating a bone related disorder, in particular a disease involving a disregulated bone homeostasis.

In an embodiment, the invention relates to the use of a nucleic acid comprising a nucleic acid sequence which is at least 65%, preferably at least 75%, preferably at least 85% and preferably at least 95% and preferably at least 99% identical to SEQ ID NO: 1, 5 or 7, or the complement thereof, for the preparation of a medicament for preventing, treating and/or alleviating a bone related disorder, in particular a disease involving a disregulated bone homeostasis.

In an embodiment, the invention relates to a method for preventing, treating and/or alleviating a bone related disorder, in particular a disease involving a disregulated bone homeostasis, comprising administering to an individual in need of such treatment a therapeutically effective amount of a nucleic acid comprising a nucleic acid sequence which is at least 65%, preferably at least 75%, preferably at least 85% and preferably at least 95% and preferably at least 99% identical to SEQ ID NO: 1, 3, 5 or 7 SEQ ID NO: 9 or 10, or of the complement thereof.

In an embodiment, the invention relates to a method for preventing, treating and/or alleviating a bone related disorder, in particular a disease involving a disregulated bone homeostasis, comprising administering to an individual in need of such treatment a therapeutically effective amount of a nucleic acid comprising a nucleic acid sequence which is at least 65%, preferably at least 75%, preferably at least 85% and preferably at least 95% and preferably at least 99% identical to SEQ ID NO: 1, 5 or 7, or of the complement thereof.

In an embodiment, the invention relates to the use of a nucleic acid specifically hybridizing to the nucleotide sequence as defined in SEQ ID NO: 1, 3, 5 or 7 or SEQ ID NO: 9 or 10, or the complement thereof, for the preparation of a medicament for preventing, treating and/or alleviating a bone related disorder, in particular a disease involving a disregulated bone homeostasis.

In an embodiment, the invention relates to a method for preventing, treating and/or alleviating a bone related disorder, in particular a disease involving a disregulated bone homeostasis, comprising administering to an individual in need of such treatment a therapeutically effective amount of a nucleic acid specifically hybridizing to the nucleotide sequence as defined in SEQ ID NO: 1, 3, 5 or 7 or SEQ ID NO: 9 or 10, or of the complement thereof.

As detailed elsewhere in the specification, in an example, a nucleic acid specifically hybridizing to the nucleotide sequence as defined in SEQ ID NO: 1, 3, 5 or 7, or SEQ ID NO: 9 or 10, or to the complement thereof, may be at least 10 nucleotides in length, and in particular longer nucleic acid molecules are contemplated, i.e. of about 15, 20, 25, 30, 40, 50, 75, 100, 200 or even more nucleotides.

As further detailed elsewhere in the specification, in another example, a nucleic acid specifically hybridizing to the nucleotide sequence as defined in SEQ ID NO: 1, 3, 5 or 7, or SEQ ID NO: 9 or 10, or to the complement thereof, may be at least 15 nucleotides in length and may be liable to act as a primer for specifically amplifying a nucleic acid of the present invention, or a part thereof. It is to be understood that said primers can be shorter, e.g. 10, 11, 12, 13, or 14 nucleotides, or longer, e.g. 16, 17, 18, 19, 20, 25, or 30 nucleotides.

Accordingly, in an embodiment, the invention relates to the use of a nucleic acid molecule specifically hybridizing to the nucleotide sequence as defined in SEQ ID NO: 1, 3, 5 or 7 or SEQ ID NO: 9 or 10, or the complement thereof, wherein said nucleic acid molecule is at least 15 nucleotides in length and is capable of specifically amplifying (liable to act as a primer for specifically amplifying) a nucleic acid of the present invention or a part thereof, for the preparation of a medicament for preventing, treating and/or alleviating a bone related disorder, in particular a disease involving a disregulated bone homeostasis.

In an embodiment, the invention relates to the use of an isolated polypeptide encodable by the nucleic acids of the present invention, or a variant or a derivative thereof, or an immunologically active and/or functional fragment thereof, for the preparation of a medicament for preventing, treating and/or alleviating a bone related disorder, in particular a disease involving a disregulated bone homeostasis.

In an embodiment, the invention relates to a method for preventing, treating and/or alleviating a bone related disorder, in particular a disease involving a disregulated bone homeostasis, comprising administering to an individual in need of such treatment a therapeutically effective amount of an isolated polypeptide encodable by the nucleic acids of the present invention.

In an embodiment, the invention relates to the use of an isolated polypeptide having (comprising) an amino acid sequence as given in SEQ ID NO:2, 4, 6 or 8 or SEQ ID NO: 11, or a variant or a derivative thereof, or an immunologically active and/or functional fragment thereof, for the preparation of a medicament for preventing, treating and/or alleviating a bone related disorder, in particular a disease involving a disregulated bone homeostasis.

In an embodiment, the invention relates to the use of an isolated polypeptide having (comprising) an amino acid sequence as given in SEQ ID NO:2, 6 or 8, or a variant or a derivative thereof, or an immunologically active and/or functional fragment thereof, for the preparation of a medicament for preventing, treating and/or alleviating a bone related disorder, in particular a disease involving a disregulated bone homeostasis.

In an embodiment, the invention relates to a method for preventing, treating and/or alleviating a bone related disorder, in particular a disease involving a disregulated bone homeostasis, comprising administering to an individual in need of such treatment a therapeutically effective amount of an isolated polypeptide having (comprising) an amino acid sequence as given in SEQ ID NO:2, 4, 6 or 8 or SEQ ID NO: 11, or a variant or a derivative thereof, or an immunologically active and/or functional fragment thereof.

In an embodiment, the invention relates to a method for preventing, treating and/or alleviating a bone related disorder, in particular a disease involving a disregulated bone homeostasis, comprising administering to an individual in need of such treatment a therapeutically effective amount of an isolated polypeptide having (comprising) an amino acid sequence as given in SEQ ID NO:2, 6 or 8, or a variant or a derivative thereof, or an immunologically active and/or functional fragment thereof.

In an embodiment, the invention relates to the use of an antibody specifically recognizing a polypeptide encodable by the nucleic acids of the present invention, or a specific epitope of said polypeptide, for the preparation of a medicament for preventing, treating and/or alleviating a bone related disorder, in particular a disease involving a disregulated bone homeostasis.

In an embodiment, the invention relates to a method for preventing, treating and/or alleviating a bone related disorder, in particular a disease involving a disregulated bone homeostasis, comprising administering to an individual in need of such treatment a therapeutically effective amount of an antibody specifically recognizing a polypeptide encodable by the nucleic acids of the present invention, or a specific epitope of said polypeptide.

The term “diseases involving a disregulated bone homeostasis” refers herein to diseases which result directly or indirectly from an impaired bone homeostasis. Examples of such diseases include but are not limited to bone resorption disorders, osteoporosis, osteopetroses, arthritides, periodontal disease, Paget's disease, periprosthetic bone loss or osteolysis, hypercalcemia of malignancy, fibrous dysplasia, cancer-induced bone loss, etc.

In another referred embodiment the present invention relates to a composition comprising a substantially purified nucleic acid, polypeptide or antibody according to the present invention for preventing, treating and/or alleviating diseases involving a disregulated bone homeostasis and possibly in conjunction with a suitable carrier. Suitable carriers for adding to the nucleic acids, polypeptides or antibodies of the present invention are well known in the art.

Accordingly, the present invention provides a pharmaceutical composition for preventing, treating and/or alleviating a bone-related disorder, in particular a disease involving a disregulated bone homeostasis, comprising a substantially purified nucleic acid, polypeptide or antibody according to the present invention, possibly in conjunction with a suitable carrier.

In a preferred embodiment, the present invention provides polypeptides according to the present invention, including protein fusions, or fragments thereof, for regulating bone homeostatis or for preventing, treating and/or alleviating diseases involving a disregulated bone homeostasis.

In another embodiment, the present invention contemplates a method for preventing, treating and/or alleviating a bone-related disorder, in particular diseases involving the disregulation of bone homeostasis comprising the use of a molecule which allows interfering with the expression of a polynucleotide or a polypeptide as described herein, in a subject.

For example, regulation of a balance between bone formation and bone resorption may occur as a direct result of administering polypeptides to mammalian, preferably human, cells. Delivering compositions containing the polypeptide of the invention to target cells may occur via association via heterologous polypeptides, heterologous nucleic acids, toxins, or pro-drugs via hydrophobic, hydrophilic, ionic and/or covalent interactions.

In another embodiment, the present invention contemplates a method for preventing, treating and/or alleviating a bone-related disorder, in particular diseases involving a disregulated bone homeostasis comprising:
(a) introducing a sample nucleic acid or an expression vector comprising a sample nucleic acid according to the present invention in a desired target cell, in vitro or in vivo,
(b) expressing said nucleic acid, and,
(c) regulating bone formation and/or bone resorption by the products expressed by said nucleic acid or the product of said expression vector.

In one preferred embodiment the present invention provides a gene therapy method for preventing, treating and/or alleviating a bone-related disorder, in particular diseases involving a disregulated bone homeostasis. The gene therapy methods relate to the introduction of nucleic acid sequences into an animal to achieve expression of a polypeptide of the present invention. This method requires a nucleic acid, which codes for a polypeptide of the invention that is operatively linked to a promoter or any other genetic element necessary for the expression of the polypeptide in the target tissue. Such gene therapy and delivery techniques are known in the art, see, for example, EP-A-0 707 071.

In one embodiment, the nucleic acid of the invention is delivered as a naked polynucleotide. The term naked nucleic acid refers to sequences that are free from any delivery vehicle that acts to assist, promote or facilitate entry into a cell, including viral sequences, viral particles, liposome formulations, lipofectin or precipitating agents and the like. The naked nucleic acids can be delivered by any method known in the art, including, but not limited to, direct needle injection at the delivery site, intravenous injection, topical administration, catheter infusion, and so-called “gene guns”.

In another embodiment, the nucleic acids of the present invention may be delivered with delivery vehicles such as viral sequences, viral particles, liposome formulations, lipofectin or precipitating agents and the like. Viral vectors that can be used for gene therapy applications include, but are not limited to, a herpes virus vector, a baculovirus vector, a lentivirus vector, a retrovirus vector, an alphavirus vector, an adeno-associated virus vector or an adenoviral vector or any combination thereof. In a preferred embodiment, viral vectors used are replication deficient, for example such as described for adenoviral vectors in WO99/64582.

Delivery of the nucleic acids into a subject may be either direct, in which case the subject is directly exposed to the nucleic acid or nucleic acid-carrying vectors, or indirect, in which case cells are first transformed with the nucleic acids in vitro, and then transplanted into the subject. These two approaches are known, respectively, as in vivo or ex vivo gene therapy and are well described. In addition, the polypeptides according to the invention can be used to produce a biopharmaceutical. The term “biopharmaceutical” relates to a recombinantly or synthetically produced polypeptide or protein. Means to recombinantly or synthetically produce polypeptides or proteins are well known in art, such as for example described in Sambrook et al. (1989). Said biopharmaceutical can be applied in vivo, such as for example intravenously or subcutaneously. Alternatively, said biopharmaceutical can be applied in vivo, such as for example by isolating cells of a subject, after which the cells are treated with said biopharmaceutical. Subsequently, said treated cells are re-introduced into said subject.

In a more preferred embodiment, the present invention provides a gene therapy method for preventing, treating and/or alleviating a bone-related disorder, in particular diseases involving a disregulated bone homeostasis comprising the use of the vectors according to the present invention.

Cells into which nucleic acids or polypeptides of the present invention can be introduced, for example for therapeutic purposes, encompass any desired available cell type, including but not limited to mesenchymal cells, progenitors of mesenchymal cells, and various stem cells, in particular mesenchymal stem cells.

In a preferred embodiment, the invention provides a method for preventing, treating and/or alleviating diseases involving a disregulated bone homeostasis (bone-related disorders) comprising the use of a molecule, which allows interfering with the expression of a polynucleotide and/or expression and/or functional activity of a polypeptide of the present invention in a subject in need of such a treatment. Such molecule may comprise, for example, specific antibodies against the polypeptides of the present invention (PLEKHM1 polypeptides) and functional fragments derived from such antibodies, antisense RNA and DNA molecules to the nucleic acids of the present invention, small interfering RNA molecules, ribozymes that function to inhibit translation of the polypeptides of the present invention, dominant negative forms of the polypeptides of the present invention, and small molecules which bind to polypeptides of the present invention and interfere with the activity of the polypeptides of the present invention.

Accordingly, the present invention also provides the use of a molecule, which allows interfering with the expression of a polynucleotide and/or expression and/or functional activity of a polypeptide of the present invention in a subject for the preparation of a medicament for preventing, treating and/or alleviating a bone-related disorder, in particular a disease involving a disregulated bone homeostasis.

Accordingly, the present invention relates to a cell, in which the polynucleotide sequences comprising the nucleic acids sequences as described herein have been introduced. It will be understood that said cell could be used as a medicament, in that said cell could be introduced in a subject suffering from pathologies related to the disturbance of bone homeostasis. Repopulating with said cells will be beneficial to the subject.

It will be understood that the present invention relates to a transgenic non-human animal comprising one or more copies of a nucleic acid of the present invention stably integrated into the genome of said animal, or an animal comprising regulatory elements that modulate the expression of a nucleic acid of the present invention.

A gene can be knocked-out by various means, therefore a preferred embodiment of the present invention pertains to a knock-out non-human animal comprising a deletion of one or two alleles encoding a nucleic acid of the present invention, or the deletion of one or more exons of said nucleic acid, or an animal comprising a targeted mutation in the genomic region, including regulatory sequences, comprising any of the nucleic acid sequences of the present invention. In general, a knock-out will result in the ablation of the function of the particular gene.

An even more preferred embodiment of the present invention pertains to the use of a transgenic or knock-out non-human animal according to the present invention as a model system for bone formation/bone resorption or bone homeostasis.

In another embodiment, the present invention provides antibody-based therapies for regulating bone formation/bone resorption or bone homeostasis in a desired target cell, in vitro, in vivo or ex vivo. Antibody-based therapies involve administering of anti-polypeptide or anti-polynucleotide antibodies to a mammalian, preferably human, cell. Methods for producing anti-polypeptide and anti-polynucleotide antibodies are known in the art. Such antibodies may be provided in pharmaceutically acceptable compositions as known in the art.

In a preferred embodiment, the present invention provides the nucleic acids, polypeptides or antibodies of the present invention for use as a medicament (both for treatment as for diagnosis of diseases). Said treatment according to the present invention refers to preventing, treating and/or alleviating diseases involving a disregulated bone homeostasis as defined above and below.

### Diagnosis

In another preferred embodiment, the present invention relates to the use of a nucleic acid, a polypeptide or antibody according to the present invention for diagnosing a pathological condition or a susceptibility to a pathological condition related to a disregulated bone homeostasis and for detecting disturbances associated with bone formation/bone resorption. As it will be understood from the invention, the diagnosis for bone disorders or a susceptibility thereto can also involve prenatal diagnosis.

In another embodiment the present invention relates to the use of a nucleic acid, a polypeptide or antibody according to the present invention for the preparation of a diagnostic kit for detecting a pathological condition or a susceptibility to a pathological condition related to a disregulated bone homeostasis.

Accordingly, the present invention also relates to a kit for diagnosing a bone-related disorder, in particular a pathological condition or disease involving a disregulated bone homeostasis, or a predisposition to a bone-related disorder in a subject, comprising a nucleic acid, a polypeptide or antibody according to the present invention. Said kit for the diagnosis of a pathological condition or a susceptibility to a pathological condition related to a disregulated bone homeostasis in a subject preferably comprises a nucleic acid, a probe or primer, a polypeptide and/or an antibody according to the present invention possibly in conjunction with suitable buffers, means for detection or detection format parts (such as, for example, solid carriers, e.g. membranes). Suitable formats and technologies for designing diagnostic kits on the basis of the above are well known in the art. Preferred formats include any type of micro-array format known in the art.

In a further embodiment, the invention provides a method for diagnosing a pathological condition or a susceptibility to a pathological condition related to a disregulated bone homeostasis in a subject comprising the steps of:
(a) determining the presence or absence of a mutation in the nucleic acid according to the present invention, including mutations in the genomic and regulatory sequences of said nucleic acid, in a biological sample,
(b) diagnosing a pathological condition or a susceptibility to a pathological condition based on the presence or absence of said mutation.

In a further embodiment, the invention provides a method for diagnosing a bone-related disorder, in particular a disease involving a disregulated bone homeostasis, or a susceptibility to a bone-related disorder in a subject comprising the steps of:
(a) determining in a biological sample from said subject the presence or absence of a mutation in a nucleic acid encoding a mammalian PLEKHM1 polypeptide or the complement thereof, including mutations in the genomic and regulatory sequences of said nucleic acid, and
(b) diagnosing the bone-related disorder or the susceptibility to the bone-related disorder in said subject based on the presence or absence of said mutation. In a further embodiment, the invention provides a method for diagnosing a bone-related disorder, in particular a disease involving a disregulated bone homeostasis, or a susceptibility to a bone-related disorder in a subject comprising the steps of:
(a) taking a biological sample from said subject,
(b) determining in said biological sample the presence or absence of a mutation in a nucleic acid encoding a mammalian PLEKHM1 polypeptide or the complement thereof, including mutations in the genomic and regulatory sequences of said nucleic acid, and
(c) diagnosing the bone-related disorder or the susceptibility to the bone-related disorder in said subject based on the presence or absence of said mutation.

In an embodiment the nucleic acid encoding a mammalian PLEKHM1 polypeptide is and/or comprises the nucleic acid according to the present invention. In an embodiment, the nucleic acid encoding a mammalian PLEKHM1 polypeptide comprises a sequence as given in SEQ ID NO: 1, 3, 5 or 7or SEQ ID NO: 9 or 10, or the complement thereof. The nucleic acid may be, for example, DNA, total RNA, mRNA, cDNA, etc.

In an even further embodiment, the present invention pertains to a method for diagnosing a pathological condition or a susceptibility to a pathological condition related to a disregulated bone homeostasis in a subject comprising the steps of:
(a) determining the presence or amount of the nucleic acid to the present invention or expression of the polypeptide to the present invention in a biological sample, and,
(b) diagnosing a pathological condition or a susceptibility to a pathological condition related to a disregulated bone homeostasis based on the presence or amount of said nucleic acid or expression of said polypeptide.

In an embodiment, the present invention provides a method for diagnosing a bone-related disorder, in particular a disease involving a disregulated bone homeostasis, or a susceptibility to a bone-related disorder in a subject comprising the steps of:
(a) determining in a biological sample from said subject the presence or amount of a nucleic acid encoding a mammalian PLEKHM1 polypeptide or determining expression of a mammalian PLEKHM1 polypeptide, and
(b) diagnosing a bone-related disorder or a susceptibility to a bone-related disorder in said subject based on the presence or amount of said nucleic acid or expression of said polypeptide.

In a further embodiment, the present invention provides a method for diagnosing a bone-related disorder, in particular a diseases involving a disregulated bone homeostasis, or a susceptibility to a bone-related disorder in a subject comprising the steps of:
(a) taking a biological sample from said subject,
(b) determining in said biological sample the presence or amount of a nucleic acid encoding a mammalian PLEKHM1 polypeptide or determining expression of a mammalian PLEKHM1 polypeptide, and
(c) diagnosing a bone-related disorder or a susceptibility to a bone-related disorder in said subject based on the presence or amount of said nucleic acid or expression of said polypeptide.

In an embodiment the nucleic acid encoding a mammalian PLEKHM1 polypeptide is and/or comprises the nucleic acid according to the present invention. In an embodiment, the nucleic acid encoding a mammalian PLEKHM1 polypeptide comprises a sequence as given in SEQ ID NO: 1, 3, 5 or 7 or SEQ ID NO: 9 or 10, or the complement thereof. The nucleic acid may be, for example, DNA, total RNA, mRNA, cDNA, etc.

In an embodiment, the mammalian PLEKHM1 polypeptide comprises an amino acid sequence as given in SEQ ID NO: 2, 4, 6 8 or SEQ ID NO: 11, or a variant or a derivative thereof, or an immunologically active and/or functional fragment thereof.

### Drug Screens

The invention further provides methods for identifying compounds or agents that can be used to treat disorders characterized by (or associated with) the disturbance of bone homeostasis, in particular bone-related disorders involving a disregulated bone homeostasis. These methods are also referred to herein as “drug screening assays” or “bioassays” and typically include the step of screening a candidate/test compound or agent for the ability to interact with (e.g., bind to) a protein of the present invention, in particular represented by SEQ ID NO: 2, 4, 6 or 8 or SEQ ID NO: 11, or any derivative, homologue, immunologically active or functional fragment thereof, to modulate the interaction of the protein of the present invention and a target molecule, and/or to modulate the expression of the nucleic acids of the present invention and/or activity of the proteins of the present invention.

For example, in one embodiment, the invention provides a method for identifying compounds or agents that can be used to treat a bone-related disorder, comprising the step of screening a candidate/test compound or agent for the ability to bind to mammalian PLEKHM1 protein, such as protein as given in SEQ ID NO: 2, 4, 6 or 8 or SEQ ID NO: 11, or any derivative, homologue, immunologically active or functional fragment thereof.

In another embodiment, the invention provides a method for identifying compounds or agents that can be used to treat a bone-related disorder, comprising the step of screening a candidate/test compound or agent for the ability modulate the expression of nucleic acids encoding mammalian PLEKHM1 polypeptide and/or activity of the mammalian PLEKHM1 polypeptide.

Candidate/test compounds or agents which have one or more of these abilities can be used as drugs to treat disorders characterized by pathological disturbance bone homeostasis, disregulated expression of the nucleic acids of the present invention and/or disregulated functional activity of the proteins of the present invention, in particular disregulated bone homeostasis. Candidate/test compounds such as antibodies, small molecules, e.g., small organic molecules and peptides, and other drug candidates can be obtained, for example, from combinatorial and natural product libraries.

The screening for therapeutic compounds may be any of a variety of drug screening techniques known in the art.

Thus, the present invention relates to the use of the nucleic acids, the polypeptides or antibodies as described herein for drug or test compound screens directed to identify drugs, test compounds or antibodies that interfere with bone formation/bone resorption or bone homeostasis.

In one embodiment, the invention provides a drug screening assay for screening candidate/test compounds which interact with (e.g., bind to) the polypeptides or proteins of the present invention, or any variant or a derivative thereof, or an immunologically active and/or functional fragment thereof. Typically, the assays are cell-free assays which include the steps of combining the polypeptides or proteins of the present invention, variants or derivatives thereof, its catalytic or immunogenic active and/or functional fragments thereof, and a candidate/test compound, e.g., under conditions which allow for interaction of (e.g., binding of) the candidate/test compound to the polypeptide or protein of the present invention, or any variant or a derivative thereof, or an immunologically active and/or functional fragment thereof, to form a complex, and detecting the formation of a complex, in which the ability of the candidate compound to interact with (e.g., bind to) the polypeptide or proteins of the present invention, or any variant or a derivative thereof, or an immunologically active and/or functional fragment thereof is indicated by the presence of the candidate/test compound in the complex. Formation of complexes between the protein of the present invention and the candidate compound can be quantified, for example, using standard immunoassays.

The proteins of the present invention, their catalytic or immunogenic fragments or oligopeptides thereof employed in such a test may be free in solution, affixed to a solid support, born on a cell surface, or located intracellularly.

In another embodiment, the invention provides screening assays to identify candidate/test compounds which modulate (e.g., stimulate or inhibit) the interaction (and most likely the functional activity of the proteins of the present invention as well) between a protein of the present invention and a molecule (target molecule) with which the protein of the present invention normally interacts, or antibodies which specifically recognize the protein of the present invention. Examples of such target molecules include proteins in the same signaling path as the protein of the present invention, e.g., proteins which may function upstream (including both stimulators and inhibitors of activity) or downstream of the signaling pathways of the proteins of the present invention.

Typically, the assays are cell-free assays which include the steps of combining a polypeptide or protein of the present invention, or any variant or a derivative thereof, or an, catalytic, immunologically active and/or functional fragment thereof, a protein target molecule (e.g., a ligand to a protein of the present invention, e.g. a receptor to a protein of the present invention) or a specific antibody and a candidate/test compound, e.g., under conditions wherein the presence of the candidate compound, the polypeptide or protein of the present invention, or any variant or a derivative thereof, or an, catalytic, immunologically active and/or functional fragment thereof interacts with (e.g., binds to) the target molecule or the antibody, and detecting the formation of a complex which includes the polypeptide or protein of the present invention, or any variant or a derivative thereof, or an, catalytic, immunologically active and/or functional fragment thereof and the target molecule or the antibody, or detecting the interaction/reaction of the polypeptide or protein of the present invention, or any variant or a derivative thereof, or an, catalytic, immunologically active and/or functional fragment thereof and the target molecule or antibody.

In another embodiment, the present invention provides a drug screening assay for the identification of test compounds which synergizes the activity, preferably bone formation/bone resorption or bone homeostasis of a nucleic acid according to the present invention or of a polypeptide according to the present invention, said assay comprising:
(a) providing a host cell,
(b) combining a test compound and a nucleic acid according to the present invention or of a polypeptide according to the present invention, under conditions wherein said test compound interacts with said nucleic acid according to the present invention or of a polypeptide according to the present invention,
(c) applying the product of step (b) to said host cell, and
(d) determining the induction of bone formation/bone resorption or bone homeostasis of said host cell, and thereby identifying the synergistic activity of said test compound.

In another embodiment, the present invention provides a drug screening assay for the identification of test compounds which modulate, and preferably agonize or antagonize, bone formation/bone resorption or bone homeostasis, said assay comprising:
(a) combining a test compound and a protein target molecule on a host cell, under conditions wherein said test compound interacts with said protein target molecule,
(b) determining the induction of bone formation/bone resorption or bone homeostasis of said host cell,
(c) combining the polypeptide according to the present invention, or a variant or a derivative thereof, or an immunologically active and/or functional fragment thereof, and a protein target molecule on a host cell, under conditions wherein said polypeptide, or a variant or a derivative thereof, or an immunologically active and/or functional fragment thereof, interacts with said protein target molecule, and,
(d) determining the induction of bone formation/bone resorption or bone homeostasis of said host cell,
(e) determining the difference in induction of bone formation/bone resorption or bone homeostasis of step (b) compared to step (d), and thereby identifying the test compound which modulates, and preferably antagonizes or agonizes, bone formation/bone resorption or bone homeostasis.

In particular, the present invention provides a drug screening method for the identification of test compounds which modulate the expression of a gene or genes according to the present invention, said assay comprising:
(a) providing a host cell comprising a gene corresponding to the nucleic acid according to the invention,
(b) introducing a test compound into said host cell, under conditions whereby said test compound modulates the expression of said gene, and,
(c) determining the expression of said gene, thereby identifying the test compound which modulates the expression of said gene.

Detection of complex formation can include direct quantification of the complex by, for example, measuring inductive effects of the protein of the present invention. A statistically significant change, such as a decrease, in the interaction of the protein of the present invention and target molecule (e.g., in the formation of a complex between the protein of the present invention and the target molecule) in the presence of a candidate compound (relative to what is detected in the absence of the candidate compound) is indicative of a modulation (e.g., stimulation or inhibition) of the interaction between the protein of the present invention and the target molecule. Modulation of the formation of complexes between the protein of the present invention and the target molecule can be quantified using, for example, an immunoassay.

Therefore, the present invention contemplates a drug screening assay for identifying compounds that modulate the interaction between binding partners in a complex, in which at least one of said binding partners is the polypeptide according to the present invention, or a variant or a derivative thereof, or an immunologically active and/or functional fragment thereof, and said method comprising:
(a) contacting a test compound with the complex, for a time sufficient to modulate the interaction in the complex; and thereafter
(b) monitoring said complex for changes in interactions, so that if a change in the interaction is detected, a compound that modulates the interaction is identified.

It should be clear that modulators for interaction between binding partners in a complex, when identified by any of the herein described methods, is contemplated in the invention. In particular, the present invention contemplates the product or compound identifiable by any of the herein described methods.

This invention also contemplates a competitive drug screening assays in which neutralizing antibodies capable of binding the proteins of the present invention specifically compete with a test compound for binding the protein of the present invention. In this manner, the antibodies can be used to detect the presence of any protein which shares one or more antigenic determinants with the protein of the present invention. In particular, the present invention pertains to a competitive drug screening assay comprising:
(a) competing the antibodies according to the present invention with a test compound for binding to the polypeptides, or a variant or a derivative thereof, or an immunologically active and/or functional fragment thereof, and,
(b) determining the amount of competition of said antibodies compared to said test compound.

Preferably, the invention relates to a drug screening assay for identifying a compound capable of use in the treatment of a disorder characterized by disregulated bone formation/bone resorption or bone homeostasis, said assay comprising:
(a) providing a cell comprising a nucleic acid according to the present invention, or any part thereof, or a polypeptide according to the present invention, or a variant or a derivative thereof, or an immunologically active and/or functional fragment thereof,
(b) providing the compound to be tested to the cell of step (a), under conditions which allow said compound to interact with said nucleic acid, or any part thereof, or with said polypeptide, or a variant or a derivative thereof, or an immunologically active and/or functional fragment thereof,
(c) assaying the ability of the compound of step (b) to modulate the expression of said nucleic acid, or any part thereof, or the activity or amount of said polypeptide, or a variant or a derivative thereof, or an immunologically active and/or functional fragment thereof, and,
d) identifying the compound for treating a disorder by disregulated bone formation/bone resorption or bone homeostasis.

The compounds identified according to the herein described drug screening assays can be used to treat, for example, disorders characterized by or associated with disregulated bone formation/bone resorption or bone homeostasis.

In a preferred embodiment the invention provides a drug screening assay for preventing, treating and/or alleviating diseases or disorders involving the disturbance bone formation/bone resorption or bone homeostasis, comprising:
(a) contacting the compounds to be screened with a nucleic acid according to the present invention, or a polypeptide according to the present invention, or a variant or a derivative thereof, or an immunologically active and/or functional fragment thereof, and,
(b) determining whether said compound effects an activity, preferably antagonizes said activity, of said nucleic acid or said polypeptide, or a variant or a derivative thereof, or an immunologically active and/or functional fragment thereof.

The present invention also contemplates the use of competitive drug screening assays in which neutralizing antibodies capable of binding polypeptides of the present invention specifically compete with a test compound for binding to the polypeptides or fragments thereof. In this manner, the antibodies are used to detect the presence of any peptide that shares one or more antigenic epitopes with a polypeptide of the invention. In particular, a screening assay for identifying antibodies that modulate the expression or functional activity of the polypeptides of the present invention, or a variant or a derivative thereof, or an immunologically active and/or functional fragment thereof, said method comprising:
(a) providing a cell comprising the polypeptide of the present invention, or a variant or a derivative thereof, or an immunologically active and/or functional fragment thereof,
(b) determining the expression and/or activity of said polypeptide, or a variant or a derivative thereof, or an immunologically active and/or functional fragment thereof,
(c) providing an antibody of the present invention, to the cell of (a), under conditions that said antibody can interact with said polypeptide, or a variant or a derivative thereof, or an immunologically active and/or functional fragment thereof,
(d) determining the modulation of expression and/or activity of said polypeptide, or a variant or a derivative thereof, or an immunologically active and/or functional fragment thereof after said antibody has bound said polypeptides, or a variant or a derivative thereof, or an immunologically active and/or functional fragment thereof.

The assays described herein include but are not limited to an Enzyme Linked Immunosorbent Assays (ELISA) or cell based Enzyme Linked Immunosorbent Assays (CELISA). Such assays allow screening for nucleic acid fragments, polypeptides, and therapeutic compounds using libraries of said compounds or molecules that influence or regulate the expression or function of the polypeptides subject of this invention. The expression of the polypeptides subject of this invention may be influenced, induced or inhibited by a target or test compound. In particular cases, said target or test compound is the expression product of a gene that is introduced into an acceptor cell. Said gene may be derived, for example, from a gene expression library. Said acceptor cells include, but are not limited to, human cells. Said gene expression libraries include, but are not limited to, adenoviral expression libraries.

In addition, the invention also relates to the product or compound identifiable by any of the herein described methods.

Also, the present invention contemplates a method for the production of a composition comprising the steps of admixing a compound identifiable by the assay as described herein with a pharmaceutically acceptable carrier.

It will be clear that the present invention contemplates a composition comprising the product or compound identifiable by any of the herein described methods.

Moreover, the present invention contemplates the use of the product or compound identifiable by any of the herein described methods as medicament.

The disclosure of all patents, publications (including published patent publications), and database accession numbers and depository accession numbers referenced in this specification are specifically incorporated herein by reference in their entirety to the same extent as if each such individual patent, publication, and database accession number, and depository accession number were specifically and individually indicated to be incorporated by reference.

It is to be understood that the following examples are meant to illustrate the embodiments of the present invention and are in no way to be construed as limiting the present invention. The invention may be practiced other than as particularly described and still be within the scope of the accompanying claims.

### Examples

### Example 1

### Localization of the Gene Causing the Osteopetrotic Phenotype in the Incisors Absent (ia) Rat on Chromosome 10q32.1

In this example, a 4.7 cM region on rat chromosome 10q32.1 was delineated in which a gene responsible for the osteopetrotic phenotype of the ia rat is located.

### Introduction

Many of the insights into the factors that regulate the differentiation and activation of osteoclasts are gained from different osteopetrotic animal models. Although bone resorption is reduced in each osteopetrotic model, a considerable variation in osteoclast number, ultrastructure and enzyme content is demonstrated. This heterogeneity among the various osteopetrotic animals depends on where the osteoclast development and activation is intercepted.

In the rat, four models of osteopetrosis, arisen from spontaneous mutations in different genes are described: (1) the toothless (tl) rat, (2) the microphthalmia blanc (mib) rat, (3) the osteopetrotic (op) rat and (4) the incisors absent (ia) rat. For two of them, the underlying genetic defect is identified. A frameshift mutation in the Csf-1 gene causes osteopetrosis in the tl rat (Van Wesenbeeck et al. 2002 Proc Natl Acad Sci USA 99(22): 14303-8; Dobbins et al. 2002 Biochem Biophys Res Commun 294(5):1114-20) and a large genomic deletion encompassing the 3′ half of the mi gene is responsible for the phenotype in the mib rat (Weilbaecher et al. 1998 J Exp Med 187(5):775-85). The gene responsible for the op rat is localized on chromosome 10 (Dobbins et al. 2002 J Bone Miner Res 17(10):1761-7; Remmers et al. J Bone Miner Res 11 (12):1856-61).

The incisors absent rat, first described in 1941 (Greep 1941 J Heredity 32:397-398) exhibits a generalized skeletal sclerosis and delay of tooth eruption that is inherited in an autosomal recessive manner, features also found in some other osteopetrotic mutations. The ia rat played a particularly important historical role in our modern understanding of some of the most basic concepts in skeletal biology. In 1973, Marks used the ia rat to carry out the first definitive studies demonstrating that the increased bone mass in osteopetrosis is due to impaired bone resorption and not to an overproduction of bone (Marks 1973 Am J Anat 138:165-190). One feature of the ia rats is that they undergo a spontaneous permanent remission between 30 and 50 days after birth. One hundred days later they are indistinguishable from normal littermates in most features except for a reduced body weight. Pursuing the question of the cellular basis for the defect, Marks went on to demonstrate that it was intrinsic to the osteoclast by curing ia mutants with transplants of normal bone marrow cells, thus simultaneously demonstrating that the osteoclast is of hematopoietic origin. An additional contribution from studies of ia rats was the discovery that tooth eruption through the jaw required osteoclastic bone resorption (Marks 1976 J Oral Pathol 5:149-63). Many insights into bone and osteoclast biology that grew out of studies of the ia rat and other osteopetrotic models during that period were reviewed by Marks and Walker (1976 In: Bourne G H (ed) The Biochemistry and Physiology of Bone, Academic Press, New York, N.Y., USA, pp. 227-301). Although the number of osteoclasts in the ia rats is increased two- to three-fold, a defective ruffled border formation is observed, along with an extensive clear zone formation and accumulation of lysosomal enzymes. Furthermore, the increased number of small cytoplasmic vesicles in the ia/ia osteoclasts indicates a secretory dysfunction, which may reflect a malfunction of intracellular traffic and/or signaling (Reinholt et al. 1999 Exp Cell Res 251 (2):477-91).

Transient osteopetrosis in human has been described in one child. A transient dysfunction of osteoclasts—presumably in utero—and a resolution soon after birth have been observed (Monaghan et al. 1991 J Pediatr 118(2):252-6). The fact that the ia rat undergoes spontaneous remission, makes it a very interesting model to study regulators of bone density.

In this example, localization of the ia mutation by positional cloning methods to rat chromosome 10 is reported, and candidate genes for the ia mutation are identified. Osteopetrotic incisors absent (ia) rat exhibits a delay of tooth eruption associated with a generalized skeletal sclerosis. In order to elucidate the underlying genetic defect of the ia rat, an outcross between the inbred ia strain and the inbred strain Brown Norway was set up.

### Material and Methods

All animal procedures were in accordance with the Guide for the Care and Use of Laboratory Animals, published by the National Institutes of Health and were approved by the Institutional Animal Care and Use Committee of the University of Massachusetts Medical School. Mutant ia rats, which are on a Long Evans background, were obtained from inbred colonies maintained at the University of Massachusetts Medical School. Radiography within 3 days of birth was used to identify mutant animals by the failure to develop marrow cavities in the long bones. Outbreeding of animals of the ia stock was done by crossing mutant homozygote males with females of the inbred Brown Norway stock (BnSsn; Harlan Sprague Dawley, Indianapolis) to produce the obligate heterozygous F1 generation. F1 animals were then intercrossed to produce the F2 generation with one animal out of four homozygous for the ia mutation. This process could be repeated to obtain the F3 and F4 generation (FIG. 9). Genomic DNA was isolated from tail biopsies of F2 and F4 mutants and used to map the chromosomal region carrying the ia mutation. Markers, selected from the Rat MapPairs screening set (Research Genetics), were analyzed by PCR using radioactively labeled primers. After electrophoresis on polyacrylamide gels, PCR products were visualized by autoradiography. Additional markers were selected from the RAT(SHRPxBN) genetic map (Rat Genome database; http://rgd.mcw.edu/). The fluorescently labeled markers were analyzed by PCR and fragments were separated on an ABI 3100 genetic analyzer

### Results

To map the ia locus, the inbred ia strain was outcrossed with the inbred strain Brown Norway (BnSsn). The latter was selected because it is genetically distinct from Long Evans, resulting in a high percentage (76%) polymorphic marker (http://rgd.mcw.edu/tools/genomescanner). Mutant males, which are sexually fertile, were bred with BnSsn females to produce the obligate heterozygote F1 generation. Intercrossing F1 animals produced the F2 generation. Further backcrosses were performed between the F2 mutant males and BnSsn females. The F3 generation again yielded obligate heterozygote animals. Intercrossing these F3 animals yielded the F4 generation with the expected Mendelian frequency of one animal in four homozygous for the ia mutation. The breeding scheme is shown in FIG. 9. 31 mutant F2 animals and 6 mutant F4 animals were available for segregation analysis.

In order to localize the ia mutation, initially, a genome search was performed on 10 mutant F2 animals. A set of markers, approximately every 10 cM on the rat genome, was selected from the Rat MapPairs screening set. Homozygosity for the Long Evans allele was initially found in all 10 animals for one marker on rat chromosome 3p13 (D3Rat53), one marker on rat chromosome 6p11 (D6Rat46) and two neighboring markers on rat chromosome 10q, located 8.1 cM from each other (D10Rat17 and D10Rat11). However, for the regions on chromosome 3p13 and 6p11 homozygosity was not obtained after the analysis of extra animals (21 F2 and 6 F4 mutant ia/ia rats) as many recombinations were found. This was not the case with the region on chromosome 10q32.1 as only one recombination with marker D10Rat17 was observed.

Genetic analysis of extra markers, selected from the RAT(SHRPxBN) genetic map, allowed to define precisely the candidate region. Homozygosity for the ia allele in all the 37 affected animals was found for two of the markers analyzed (D10Rat18 and D10Rat84) (FIG. 10). Because these are completely linked with the disease, a maximal LOD score of +22.2 is obtained on a genetic distance of 0 cM. On the proximal side, a recombination event has occurred with D10Rat127 in mutant 4 and with both D10Rat127 and D10Rat99 in mutant 7, whereas on the distal side the disease recombines with D10Rat11 in mutant 12, 15 and 21 and with D10Rat11, D10Rat13 and D10Rat17 in mutant 23. This refined the candidate region to approximately 4.7 cM, flanked by the markers D10Rat99 and D10Rat17 (FIG. 10).

### Discussion

In this example, the localization of the ia mutation to rat chromosome 10q32.1 is described. Homozygosity for the ia allele was found for two markers (D10Rat18 and D10Rat84) in 37 F2 ia/ia animals, resulting in a positive LOD score of +22.2. Key recombinants both on the proximal and distal side could delineate a candidate region of 4.7 cM.

The ia critical region extends from the hap1 gene on the proximal side to the LOC303597 gene (similar to KIAA1636 protein) on the distal side (http://www.ncbi.nlm.nih.gov/mapview/). A large part of this 5.8 Mb segment has been sequenced and placed into two supercontigs, assembled from whole genome shotgun sequences (genbank accession numbers NW_{—}042674 and NW_{—}042675). Although various gaps still exist in this sequence, over 140 known or putative genes have been identified, based on homology data (with mouse and human) and on automated computational analysis using gene prediction methods such as GenomeScan. In order to analyze the existing gaps in this 5.8 Mb sequence, the homologous regions of this portion of rat chromosome 10 in human (http://rgd.mcw.edu/VCMAP/) were identified. Conservation between rat chromosome 10q32.1 and two regions on human chromosome 17, one on 17q21 and one on 17q23, has been observed (http://www.ensembl.org/). On human chromosome 17q21, a few clusters of genes have been conserved, while the order of other grouped genes has been reversed with regard to the position of the rat genes. A few genes on rat chromosome 10q32.1 have been conserved with human chromosome 17q23 (FIG. 11).

None of the osteopetrotic mutations that have been mapped to date are localized to this chromosomal region in rat, mouse or human (Benichou et al. 1998 Rev Rhum Engl Ed 65(12):778-87; Chalhoub et al. 2003 Nat Med 9(4):399-406; Van Wesenbeeck L et al. 2003 Am J Hum Genet 72(3):763-71; Campos-Xavier et al. 2003 Hum Genet 112(2):186-9; Cleiren E et al. 2001 Hum Mol Genet 10(25):2861-7; Kornak et al. 2001 Cell 104(2):205-15; Frattini A et al. 2000 Nat Genet 25(3):343-6; Kornak et al. 2000 Hum Mol Genet 9(13):2059-63; Sly et al. 1983 Proc Natl Acad Sci USA 80(9):2752-6; Van Wesenbeeck et al. 2002 Proc Natl Acad Sci USA 99(22): 14303-8; Dobbins et al. 2002 Biochem Biophys Res Commun 294(5):1114-20; Weilbaecher et al. 1998 J Exp Med 187(5):775-85; Dobbins et al. 2002 J Bone Miner Res 17(10):1761-7; Remmers et al. 1996 J Bone Miner Res 11(12):1856-61). Therefore, the ia gene are different from those involved in the 8 other spontaneous osteopetrotic animals. Although the sequence of this large region is not complete, several candidate genes were identified (see table 1). At first we looked for genes with a role in osteoclast function, since the primary genetic defect in the ia rat is located in the osteoclast cell lineage Reinholt et al. 1999).

TABLE 1
Positional candidate genes with a role in osteoclast function and bone homeostasis.

| Gene | | Putative function |
|---|---|---|
| ATP6N1A | ATPase, H+ transporting, lysosomal, | Acidification by the transfer of protons |
| | noncatalytic accessory protein 1a | |
| ITGB3 | Integrin beta 3 | Cell-matrix interactions (osteoclast attachment) |
| NSF | N-ethylmaleimide sensitive factor | Involved in intracellular membrane trafficking processes |
| RAB5C | Ras-associated protein rab5c | Involved in intracellular membrane trafficking processes |
| RH07 | GTP-binding protein rho7 | Involved in intracellular membrane trafficking processes |
| ARF2 | ADP-ribosylation factor 2 | Involved in intracellular membrane trafficking processes |
| ARHGAP12 | Rho GTPase activating protein 12 | Involved in intracellular membrane trafficking processes |
| SOST | Sclerostin | Involved in intracellular membrane trafficking processes |
| RAMP2 | Receptor activity-modifying protein 2 | Interaction with calcitonin receptor-like receptor |
| KBRAS2 | I-κ-B-interacting ras-like protein 2 | Involved in the NF-κ-B signaling pathway |

The resorption process requires an acidic environment, which is created in a sealed compartment between the osteoclast and the bone surface by the transfer of protons over the plasmamembrane. The ATP6N1A gene product is the al isoform subunit of the vacuolar proton pump that is involved in membrane traffic processes through the acidification of intracellular compartments. These H⁺-ATPases (or V-ATPases) are composed of 2 functional domains. The V1 domain (subunits A-H) is responsible for ATP hydrolysis, whereas the V0 domain (subunits a, c, c′, c″ and d) forms a proton pathway across the membrane (Forgac M 1999 J Biol Chem 274(19):12951-4). Three different isoforms of subunit a (a1, a2 and a3) have been identified (Toyomura et al. 2000 J Biol Chem 275(12):8760-5). The a3 isoform is induced during osteoclast differentiation and localized in the osteoclast plasma membrane, whereas the a1 isoform is constitutively expressed and localized in cytoplasmic endomembrane compartments of the osteoclasts (Toyomura et al. 2000 J Biol Chem 275(12):8760-5). The TCIRG1 gene, encoding the a3 subunit, has been shown to be involved in a subset of human autosomal recessive osteopetrosis (Frattini et al. 2000 Nat Genet 25(3):343-6; Kornak et al. 2000. Hum Mol Genet 9(13):2059-63). Deletion of the 5′ part of this gene is responsible for the phenotype of the osteosclerotic (oc) mouse (Scimeca et al. 2000. Bone 26(3):207-13). However, the severity of the human autosomal recessive form and the fact that oc mutants die around three weeks of age are clearly in contrast with the spontaneous remission of the ia rats between 30-50 days after birth. Integrins are transmembrane heterodimeric glycoproteins that mediate cell-matrix and cell-cell interactions. Osteoclasts highly express the ανβ3 integrin. Interference with this ανβ3 integrin in vitro and in vivo leads to inhibition of bone resorption (Duong et al. 2000 Matrix Biol 19(2):97-105). Furthermore, targeted disruption of the integrin beta 3 subunit in mice induces an osteosclerosis with age because of dysfunctional osteoclasts (McHugh et al. 2000 J Clin Invest 105(4):433-40). In the ia rat the osteosclerosis resolves with age and furthermore an increased level of the integrin β3 subunit was detected at the clear zone of mutant ia/ia osteoclasts, compared to normal littermates (Reinholt et al. 1999). Polarization of resorbing osteoclasts requires a variety of intracellular membrane trafficking processes. N-ethylmaleimide sensitive factor (NSF) has been found as a ubiquitous ATPase involved in membrane fusion events throughout intracellular traffic, including the terminal step of exocytosis at the plasma membrane. Together with soluble NSF attachment proteins (SNAPs), NSF actually plays the role of a chaperone by activating SNAP receptor proteins (SNAREs) so that they can participate in membrane fusion (Whiteheart et al. 2001 Int Rev Cytol 207:71-112). Ras-associated protein RAB5C, a small GTP-binding protein of the rab family, is associated with early endosomes in osteoclasts (Zhao et al. 2002 Biochem Biophys Res Commun 293(3):1060-5). GTP-binding protein RHO7, ADP-ribosylation factor 2 (ARF2) and Rho GTPase activating protein 12 (ARHGAP12) are also involved in vesicular transport (Chardin 1991 Cancer Cells 3(4):117-26; Stamnes 2002 Curr Opin Cell Biol. 14(4):428-33; Zhang et al. 2002 Int J Biochem Cell Biol 34(4):325-31). Since the ia rats have a defective intracellular traffic (Reinholt et al. 1999) we consider all these genes involved in vesicular transport, although not specific for the osteoclast, as potential candidate genes for the ia mutation. Several additional genes with a role in bone homeostasis are mapped in the critical ia region. Loss-of-function mutations in the SOST gene lead to sclerosteosis, a progressive sclerosing bone dysplasia. Sclerostin, encoded by the SOST gene, acts as a negative regulator of bone formation, most likely by inhibiting bone morphogenetic protein signaling (Brunkow et al. 2001 Am J Hum Genet 68(3):577-89; Naot et al. 2001. Endocrinology 142(5):1849-57). The receptor activity-modifying protein 2 (RAMP2) interacts with calcitonin receptor-like receptor to form a heterodimeric complex, which forms an additional receptor for adrenomedullin. This peptide hormone acts as a local regulator of bone growth (Fenwick et al. 2000 Science 287(5454):869-73). I-Kappa-B-interacting ras-like protein 2 (KBRAS2) is involved in the NF-kappa-B signaling pathway and could have a possible role in osteoclast function (Fenwick et al. 2000 Science 287(5454):869-73). Besides these functional candidate genes, genes with a currently known function may have an additional, not yet identified, role in bone metabolism. Moreover, within the ia critical region there are also at least 40 putative genes with a currently unknown function, which could play a role in bone resorption.

### Summary

Many of the insights into the factors that regulate the differentiation and activation of osteoclasts are gained from different osteopetrotic animal models. The osteopetrotic incisors absent (ia) rat, first described in 1941, exhibits a delay of tooth eruption associated with a generalized skeletal sclerosis (Greep R O 1941 An hereditary absence of the incisor teeth. J Heredity 32:397-398). Using the above-cited rat-model, the applicant has demonstrated that a mutation in a gene, which is located in the region on rat chromosome 10q32.1, is responsible for the osteopetrotic phenotype of the ia rat model.

### Example 2

### Loss of Function Mutation in the LOC303584 (PLEKHM1) Gene

Genetic analysis of additional markers and 50 additional mutant rats allowed to further narrow the ia candidate region on rat chromosome 10q32.1 to 2.2 cM between markers D10Rat205 and D10Got137, which contains at least 23 known or putative genes. Some of these genes were functional candidates as mentioned above, including integrin beta 3 (itg β3), N-ethylmaleimide sensitive factor (NSF), ADP-ribosylation factor 2 (ARF2) and Golgi SNAP receptor complex member 2 (GOSR2). Hence, it was not at all obvious which of the genes in the 2.2 cM candidate region would be responsible for the ia phenotype. Rather, finding of the actual gene carrying the ia mutation required a great amount of experimentation as described herein. Namely, mutation analysis of the genes from this 2.2 cM ia candidate region was performed by direct sequencing on cDNA obtained from normal and ia rats. Total RNA was isolated from kidney and bone tissues of normal and ia rats using TRIzol reagent (Invitrogen). cDNA synthesis was performed starting from 5 μg total RNA primed with oligo(dT) using the Superscript III first strand synthesis system (Invitrogen). PCR primers were developed to amplify the complete coding sequence of the various genes from the candidate region. The DYEnamic ET terminator cycle sequencing kit (Amersham Biosciences) was used and the PCR fragments were analyzed on an ABI 3100 genetic analyzer.

In the course of the analysis, a mutation analysis of the complete cDNA sequence of the LOC303584 gene in ia rats was performed and revealed a deletion of 1 cytosine in exon 4 of the LOC303584 gene in ia rats. This 1 bp-deletion on cDNA position 1012 (in a stretch of 6 cytosines on cDNA positions 1007-1012 in SEQ ID NO: 1) results in a frameshift followed by an additional 5 unrelated amino acids and a stop codon (FIG. 12) yielding an abnormal, highly truncated protein, of which the amino acid sequence (SEQ ID NO: 4) is illustrated in FIG. 4 (342 amino acids in the mutated protein versus 1059 amino acids in the mature protein). This deletion was homozygous in all the mutant ia rats. This deletion was also checked on genomic DNA of ia rats. The applicant thus demonstrated that the osteopetrosis seen in ia rats is due to a loss of function of the PLEKHM1 (LOC303584) gene.

### Example 3

### Expression Analysis

Expression analyses of the above-mentioned LOC303584 gene were performed and showed with a multiple rat tissue cDNA panel that the gene has a broad expression pattern, and is expressed in skeletal muscle, liver, kidney, lung, brain, testis, pancreas, heart and bone tissue. Expression of the gene in osteoclasts could also be demonstrated. In particular, expression of the gene and the existence of alternative splice variants were analyzed with a rat Multiple Tissue cDNA panel (Clontech). PCR primers were developed to amplify the complete coding sequence of the plekhm1 gene. The plekhm1 gene is expressed in all tissues tested including testis, skeletal muscle, lung, liver, spleen, brain, heart, kidney and bone (FIG. 13). No alternative splice variants were detected. Expression of the human PLEKHM1 gene (and of its transcribed pseudogene) in cultures of differentiated human osteoclast cells and in several human tissues was also demonstrated by RT-PCR and sequencing analysis.

### Example 4

### Homology Data for the plkhm1 Gene

The Pleckstrin Homology domain containing family M (with RUN domain) member 1 (plekhm1) gene has orthologues in several organisms. In comparison with the rat plekhm1 gene, the cDNA sequence of the human and mouse homologues are conserved for 83% and 93%, respectively. The rat amino acid sequence is conserved for 82.3% and 93.5% in comparison with the human and mouse PLEKHM1 protein, respectively (FIG. 14). Only in humans, one transcribed pseudogene, located at a distance of approximately 2 Mb from the PLEKHM1 gene, is observed. Sequencing analysis of the pseudogene cDNA, however, revealed numerous nucleotide substitutions, partial deletion of exon 3 including a part of the RUN domain, and a deletion of exon 4, which results in a translational frameshift and, subsequently, a premature stop codon, indicating that the pseudogene transcript is probably not functional in the same manner as the PLEKHM1 gene. Corresponding GenBank accession numbers for the above genes are: the rat plekhm1 gene, NM_{—}001009677; the mouse plekhm1 gene, NM_{—}183034; the human PLEKHM1 gene, NM_{—}014798; the pseudogene of the human PLEKHM1 gene, BC041324, AI833315 and BC034693.

### Example 5

### In Silico Analysis of the Sequence of the plekhm1 Sequence

The plekhm1 protein has been partially described as the adaptor protein 162 (AP162) protein, to be expressed in colon tissue and to function as an intracellular adaptor protein modulating apoptotic signals (Hartel-Schenk et al. 2001). Thus so far, the plekhm1 protein is described with a putative function in colon but without an indication of a role in bone metabolism. In order to elucidate the potential cellular function of the plekhm1 protein, we analyzed the sequence with several prediction programs including the InterPro database (http://www.ebi.ac.uk/interpro/), which predicts functional domains; Signal P 3.0 server (http://www.cbs.dtu.dk/services/SignalP/), which predicts the presence of a signal peptide; and TMHMM 3.0 server (http://www.cbs.dtu.dk/services/TMHMM/), which predicts the presence of transmembrane helices. These programs predicted the following features (FIG. 15).

The RUN domain (amino acids 41-182), which is organized into six conserved blocks and might play a role in multiple Ras-like GTPase signaling pathways (Callebaut et al. 2001 Trends Biochem Sci 26: 79-83; Ingley et al. 1994 J Cell Biochem 56: 436-43). The Pleckstrin Homology (PH) domain (amino acids 537-628 and 686-781) which consists of about 100 residues that occur in a wide range of proteins involved in intracellular signaling or functioning as constituents of the cytoskeleton. Amino acids 990-1043 of the rat plekhm1 protein are predicted to contain a RING finger domain with an E3 ubiquitin-protein ligase activity or a protein kinase C conserved region 1 (C1) domain which may bind to phorbol esters, diacylglycerol or RasGTP. These cysteine rich domains may be involved in the binding of zinc. Furthermore, the plekhm1 protein is predicted to be a non-secretory protein without a signal peptide or any transmembrane helices.

The RUN and PH domains may suggest a role for the plekhm1 protein in Ras-like GTPase intracellular signaling pathways (Callebaut et al. 2001 Trends Biochem Sci 26: 79-83; Ingley et al. 1994 J Cell Biochem 56: 436-43). GTPases of the Ras-like superfamily (small GTPases) participate in many biological processes. The switch between GDP-bound inactive forms and GTP-bound active forms is regulated by different classes of proteins including GDP/GTP exchange factors, GTPase-activating proteins and guanine nucleotide dissociation inhibitors. RUN domains (amino acids 41-182 of the plekhm1 protein) encode a globular structure consisting of 6 conserved blocks that are predominantly alpha fold. They are found in a number of proteins, several of which have been proven to interact with small GTPases (Janoueix-Lerosey et al. 1995 J Biol Chem 270: 14801-8; Janoueix-Lerosey et al. 1998 Eur J Biochem 252: 290-8). The Pleckstrin Homology (PH) domain (amino acids 537-628 and 686-781 of the plekhm1 protein) consists of about 100 residues and can be divided into 6 subdomains. Despite a limited amino acid homology between the PH domains, a common tertiary structure consisting of two anti-parallel beta-sheets followed by an amphiphatic C-terminal alpha-helix is observed. PH domains are found in a wide variety of proteins involved in intracellular signaling, such as in protein kinases, phospholipases, adaptor proteins and regulators of small GTPases; PH domains may further bind with phosphoinositides to target the PH-domain containing proteins to the cellular membranes; further, many cytoskeletal proteins, like spectrin and dynamin, contain PH domains for reorganization of cytoskeleton, cell polarization and chemotaxis (Cozier et al. 2004 Curr Top Microbiol Immunol 282: 49-88; Lemmon et al. 2004 Biochem Soc Trans 32: 707-11; Maghazachi et al. 2000 Int J Biochem Cell Biol 32: 931-43; Lemmon et al. 2002 FEBS Lett 513: 71-6; Ingley et al. 1994 J Cell Biochem 56: 436-43). The list of PH-containing proteins currently includes several that participate in the regulation of small GTPases. A putative role of the plekhm1 protein in small GTPase signal transduction may agree with previous results showing that several members of the small GTPase superfamily play a role in the regulation of osteoclast activity (Coxon et al. 2003 Calcif Tissue Int 72: 80-4). For example, Rab GTPases are crucial for vesicular membrane transport in both exo- and endocytosis and are therefore essential for the formation of the ruffled border in osteoclasts.

In search for homologous domains in other proteins, the PLEKHM2 protein (GenBank accession number XM_{—}290944; XP_{—}290944) is another example of an association of a PH domain with a RUN domain. This protein is only computationally predicted with currently unknown function.

### Example 6

### Mutation Analysis of the PLEKHM1 Gene in Human Patients with Osteopetrosis

Mutation analysis was performed of the entire coding sequence of the human PLEKHM1 gene in 44 patients diagnosed with various forms of osteopetrosis. Genomic DNA was isolated from whole blood using standard procedures. Primers were developed to amplify by PCR very specifically the complete coding sequence and all exon-intron boundaries of the PLEKHM1 gene. The screening for mutations in the PLEKHM1 gene using these primers was not interfered by simultaneous amplification of the pseudogene. The following primer pairs were used to amplify the individual exons of the human PLEKHM1 gene (including exon-intron boundaries and partial intronic sequences flanking the exons) on genomic DNA of the subjects (Table 2):

| Amplified | | | |
|---|---|---|---|
| exon | Primers name | Primer sequence (5′→3′) | SEQ ID NO |
| 1 | 1F | AGTAAGAGAGGCTGACCTCC | SEQ ID NO: 14 |
| | 1B | ACAGTGCCCAAGGAAGTGAGC | SEQ ID NO: 15 |
| 2 | 2F | TTGCACACGTATTTGGCACC | SEQ ID NO: 16 |
| | 2R | TCTGTTGGCTTCCTGAACAGC | SEQ ID NO: 17 |
| 3 | 3A | TTATTCCTGGTTCCTGCTAACC | SEQ ID NO: 18 |
| | 3R | AGTAACAGACAATCACATGC | SEQ ID NO: 19 |
| 4 | 4F | CAGCAGTTTTGCATTCTTCC | SEQ ID NO: 20 |
| | 4B | TGCGGTCAGACAAGAACAACC | SEQ ID NO: 21 |
| 5 | 5A | AGCTACTCAGGAGACTGGGG | SEQ ID NO: 22 |
| | 5R | AACAGAACTTCACAGCTGCC | SEQ ID NO: 23 |
| 6 | 6F | ATGCACTGCAGGGCATCAGG | SEQ ID NO: 24 |
| | 6D | AGTGAGTAGCCAGCCTGCCG | SEQ ID NO: 25 |
| 7 | 7A | ATCAGACACTGTGCACACTCC | SEQ ID NO: 26 |
| | 7B | AGCTCCCCAACATCACAGTGC | SEQ ID NO: 27 |
| 8 | 8F | AAGTGAGCAGGTCATGTGGC | SEQ ID NO: 28 |
| | 8R | TGCATGTGCACGAGTGCCTGC | SEQ ID NO: 29 |
| 9 | 9F | AGGCCGAATTACACTCCTAGC | SEQ ID NO: 30 |
| | 9R | TGCAGAAAAGCTACAGACTGC | SEQ ID NO: 31 |
| 10 | 10F | CCATGGCACCTCAGTGCAGC | SEQ ID NO: 32 |
| | 10R | ATGGGCTGCCCCAACAACTGC | SEQ ID NO: 33 |
| 11 | 11F | GRTGTGACGTATGGGTAAGC | SEQ ID NO: 34 |
| | 11B | TAGGCAAACCCAGCCGGGATGG | SEQ ID NO: 35 |

Direct sequencing on the obtained PCR amplification products was performed using the above primers. Additional primers were used to sequence the PCR products containing exon 5 (primer 5B, 5′-cTgTTgTcTTcTTTcccAAc-3′, SEQ ID NO: 38) and exon 6 (primer 6A, 5′-AgggcAcAcAgTTTgAcTgg-3′, SEQ ID NO: 39; and primer 6B, 5′-AcATTTcAgAgccTccAAgg-3′, SEQ ID NO: 40). The DYEnamic ET terminator cycle sequencing kit (Amersham Biosciences) was used and the PCR fragments were analyzed on an ABI 3100 genetic analyzer.

Two members of a family with a G to A transition at position +1 of the donor splice site of intron 2 of the PLEKHM1 gene were identified. They were both homozygous for this splice site mutation (IVS2+1 G to A), which is consistent with the autosomal recessive nature of the disease and the consanguineous marriage of their parents (FIG. 16A). Sequencing analysis of DNA from the parents of these two members (siblings) and from a brother of these siblings showed heterozygosity for this mutation (FIG. 16A). The mutation was not found in 50 control chromosomes. The analysis was performed using primers 2F and 2R for PCR amplification on genomic DNA and sequencing of exon 2 and the flanking intronic regions.

One of the two members homozygous for the above mutation in the of PLEKHM1 gene was a girl with the following clinical picture. The girl was followed since 1997 (at that time she was 7 years old) for genua valga with suspicion of metaphysic dysplasia. Radiographic appearance showed an “Erlenmeyer flask” deformity of the distal femora (FIG. 17A). She was diagnosed as having the intermediate type of osteopetrosis. At that time the genua valga was her only complain. In December 2000 she underwent a bilateral hemi-epiphysiodesis with cannulated screws on the distal femur and proximal tibia. There were no complications. In June 2001 she presented with pain in the left fossa iliaca and groin. There was no fever or vomiting. She had pain of the left thigh and decreased rotation and pain of the left hip. An ultrasound showed bilateral hydrops of the hip joint. Bone scintigraphy was performed and showed no necrosis. She was diagnosed as having the intermediate type of osteopetrosis. After 1 day the symptoms of pain in the left hip had been resolved spontaneous. In February 2002 the alignment of her legs was normal and the screws were removed. She was followed with radiographs every 6 months (FIG. 17B). There were no complaints. The last follow-up radiograph was taken in January 2004 (FIG. 17C). There was a little narrowing of the joint space of the left hip, but no pain. In October 2004, she was 14 years of age and she presented with a history for 14 days of pain in the left leg during walking. The pain was relieved with ibuprofen. She had a normal alignment and normal leg-length. Pain during mobilisation of the left hip and decreased endorotation of 0° and exorotation of 10°. Flexion was 90°, abduction was 15°, both with severe pain. Radiographs showed a decreased joint-space in the left hip and a chondrolysis of the left hip (FIG. 17D). We restricted her activities and performed an infiltration with hyaluronate acid in the left hip joint.

No clinical or radiological symptoms were found in the mutated sibling of this patient, who was, however, only 5 years old.

### Example 7

### Effects of the IVS2+1 G to A Mutation on mRNA

To study the effect of the G to A mutation at position +1 of the donor splice site of intron 2 of the PLEKHM1 gene (IVS2+1 G to A) at the RNA level, an RT-PCR assay spanning exons 2 and 3 was designed so that only the PLEKHM1 gene and not the pseudogene was amplified. The primers used for the RT-PCR amplification and sequencing were the primer 2A (5′-TccgTgAAggccTTgcAgAAgc-3′, SEQ ID NO: 36) and primer 3B (5′-TgggcTggTAgTAcTcATgcAAgc-3′, SEQ ID NO: 37). Total RNA was isolated from cultured lymphoblasts and cDNA synthesis was performed starting from 5 μg total RNA primed with oligo(dT) using the Superscript III first strand synthesis system (Invitrogen). cDNA amplification was performed using a forward primer within exon 2 and a reverse primer within exon 3 to amplify only the PLEKHM1 gene and not the pseudogene.

RT-PCR on total RNA isolated from cultured lymphoblasts produced a 382 base pair product in a healthy control. This control was not related to the patient with osteopetrosis described in example 6. In contrast, RT-PCR on total RNA isolated from cultured lymphoblasts from said osteopetrotic patient, who is homozygous for the IVS2+1 G to A mutation, did not result in this 382 base pair product. Rather, two larger products were observed (FIG. 16B). Direct sequencing of these products revealed that one larger product contained a 95 base pair portion of part of intron 2 and the other one contained a 262 base pair portion of intron 2, due to the presence of cryptic splice donor sites in intron 2. Accordingly, the IVS2+1 G to A mutation leads to the production of two different PLEKHM1 transcripts with cDNA sequence containing either a 95 bp portion of intron 2 (FIG. 21, SEQ ID NO: 9) or a 262 bp portion of intron 2 (FIG. 22, SEQ ID NO: 10). These transcripts encode a highly truncated mutant PLEKHM1 protein of 99 amino acids (FIG. 23, SEQ ID NO: 11). As expected, RT-PCR on total RNA from carriers heterozygous for the IVS2+1 G to A mutation resulted in three fragments, corresponding to the normal and the two aberrant transcripts. Both alternative splicing products resulted in a premature stop codon and a highly truncated and thus likely non-functional gene product (FIGS. 16B and 23).

In conclusion, the present inventors identified a PLEKHM1 mutation as the primary cause of a case of autosomal recessive intermediate osteopetrosis. To date, only the CLCN7 gene had been associated with this form of osteopetrosis (Campos-Xavier et al. 2003 Hum Genet 112: 186-9; Frattini et al 2003 J Bone Miner Res 18: 1740-7). The mild phenotype of this form and the spontaneous recovery of the osteopetrosis in the ia rats indicate that inactivation and/or down-regulation of the plekhm1 protein might not completely inhibit the bone resorption process or that some compensatory mechanisms may exist. The present findings greatly enhance molecular diagnostics and genetic counselling in human osteopetroses and are useful in therapeutic applications involving the PLEKHM1 gene.

### Example 8

### Expression of EGFP-PLEKHM1 Fusion Protein in HEK Cells

Human PLEKHM1 cDNA was amplified by PCR starting from the IMAGE cDNA clone 5535600 (RZPD) and cloned in frame into the pEGFP-N1 vector (Clontech). HEK293T cells were grown in DMEM medium supplemented with 10% foetal bovine serum (Invitrogen). Before transfection the cells were plated on cover slips for 24 hours with a density of 2.105 cells/well. Cells were transfected with 50 ng of the purified construct using lipofectamine reagent (Invitrogen) according to the manufacturer's instructions. After 24 hours, the expression of the EGFP-PLEKHM1 fusion protein was examined in cells using ZEISS CLSM 510 confocal fluorescence microscope equipped with an argon laser (excitation: 488 nm). The EGFP-PLEKHM1 fusion protein showed intracellular localization with diffuse cytoplasmic expression and appearance of some abnormally enlarged vesicles (FIG. 18). Interestingly, similar large vesicular structures were seen upon over expression of some Rab GTPases, including Rab7, Rab22a, and Rab5 (Bucci et al. 2000 Mol Biol Cell 11: 467-80; Mesa et al. 2001 J Cell Sci 114: 4041-9; Barbieri et al. 1996 Arch Biochem Biophys 326: 64-72), which may suggest a role for small GTPases and for the PLEKHM1 protein in the fusion of endocytic structures and vesicular transport (e.g., constitutively active mutants).

### Example 9

### Antibody Production

An anti-peptide antibody production against the rat protein was started up (Eurogentec). Two peptides of the rat sequence were selected and the mix of these two peptides was injected in two Pasteurella multocida, pathogen-free (SPF) rabbits. Immunization of two rabbits continued for three months following a standard immunization protocol. In particular, two SPF rabbits were immunized against a mix of two peptides of the rat plekhm1 protein following a standard immunization protocol (Eurogentec). These peptides were PTSPKGKSWISEDDFC and LQKVRPQQEEEWVNIC, located respectively at amino acid position 431-445 and 626-640 of the rat plekhm1 protein (SEQ ID NO: 2). Affinity purification of 5 ml antiserum against each of the peptides separately was performed to obtain purified antibodies against the rat plekhm1 protein (named anti-plekhm1A and anti-plekhm1B, respectively).

Western blotting with both antibodies on lysates of cells transfected with the plekhm1-EGFP construct detected a strong band corresponding to the plekhm1 and the GFP protein showing the specificity of the antibodies. Results of Western Blots using the antiserum of the rabbits and a goat anti-rabbit secondary antibody labelled with horseradisch peroxidase, showed a clear band. This band was not detected in the negative controls. This example indicates that antibodies can be prepared against the rat PLEKHM1 protein.

### Example 10

### Immuno-Histochemistry Using the Anti-plekhm1 Antibodies

To determine the expression of the plekhm1 protein in bone tissues, immuno-histochemistry was performed on bone slides. Tibia and femurs were dissected from wild-type rats at 1 day and 2 weeks of age. The bone tissues were fixed with formaldehyde, de-mineralized and embedded in paraffin. 3 micrometer sections were mounted on slides, baked overnight and then de-paraffinised using standard procedures. The slides were blocked with 3% hydrogen peroxide and subsequently with normal goat serum (20% dilution in 1×PBS). Incubation with the primary antibodies of both purifications against peptide 1 and 2 (dilution of 1/50 and 1/200 in normal goat serum, respectively) was done overnight at 4 degrees. Incubation of the biotin-labelled secondary antibody (goat anti-rabbit) was performed for 30 minutes at 37° C. After incubation of the Avidin-Biotin complex for 30 minutes, the chromogenic substrate diaminobenzidine was added for 5 minutes. Counterstaining with hematoxyline-eosin was performed to detect the nuclei and to show the general morphology. The specificity of the antibodies was tested by neutralizing the immuno-staining by addition of the corresponding peptides and showed that the antibodies are specific (FIG. 19B top without peptide and bottom with peptide).

As shown in FIG. 19A, an intense staining of osteoclasts (arrow) is observed in wild-type rats. There is also staining of osteoblasts, osteocytes, megakaryocytes in the marrow and chondrocytes. An identical pattern was seen with both purifications of the anti-plekhm1 antibody and no differences in staining were observed between the bones from 1 day and two week old animals.

### Example 11

### Immuno-Fluorescence Microscopy in Cultured Osteoclast Cells Using the Anti-plekhm1 Antibodies

To determine the sub-cellular localization of the plekhm1 protein, immuno-fluorescence microscopy analysis was carried out. Bone marrow or spleen cells were differentiated on glass cover slips to multinucleated osteoclasts by adding CSF1 and RANKL, essentially as follows. Bone marrow of long bones of normal littermates was flushed out with cold PBS. Because there is little marrow space in the long bones of ia mutants, spleen cells were used to prepare osteoclasts. The cells were carefully laid over Histopaque-1077 medium (Sigma Diagnostics) and centrifugation at 1800 rpm for 30 minutes at 10° C. caused the mononuclear cells to form a distinct layer at the plasma-histopaque interface. After washing these mononuclear cells with cold PBS, the cell pellet was re-suspended in complete medium (α-MEM with 10% foetal bovine serum, Invitrogen). These cells were incubated in complete medium containing 15 ng/ml CSF1 at 37° C. in a humidified atmosphere of 95% air and 5% CO₂ for 24 hours. The non-adherent cells were collected and cultured at a concentration of 5×10⁵ cells on glass cover slips in each well of a 12 well plate in complete medium containing 75 ng/ml CSF1 and 30 ng/ml RANKL (PeproTech). The bone marrow cells and the spleen cells differentiated to big multinucleated cells after approximately 1 week.

The cells were fixed for 50 minutes at room temperature with 3.7% formaldehyde in PBS, washed two times with PBS and permeabilised for 2 minutes with 0.1% Triton X-100 in PBS. After one wash step with 0.5% bovine serum albumin (BSA, Sigma) in PBS, the primary antibodies were incubated for 1 hour at 37° C. The anti-plekhm1 antibodies of the purifications against peptide 1 and 2 were both used at a 1/50 dilution in 0.5% BSA/PBS. The cells were washed three times with 0.5% BSA/PBS and then incubated with the Alexa fluor 488 labelled donkey anti-rabbit secondary antibody (Molecular Probes) for 1 hour at 37° C., followed by one wash step with 0.5% BSA/PBS. Subsequently, the nuclei were stained by adding 5 μg/ml DAPI (Molecular Probes) in 0.1% Triton/PBS for 5 minutes at room temperature. After the final wash steps with PBS, the cover slips were mounted on glass slides in Prolong Antifade reagent (Molecular Probes) to reduce the photo-bleaching of the fluorophores. The specificity of the anti-plekhm1 antibodies was tested by neutralizing the immunofluorescence signal by the addition of the corresponding peptides (150 μg/well). The slides were viewed with fluorescence microscopy.

The two affinity purifications of the anti-plekhm1 antibody stained the cells in a similar pattern and both the mononuclear cells and the multinucleated osteoclast cells were labelled. Similar to the immuno-histochemistry, peptide neutralization confirmed the specificity of the labelling. As shown in FIG. 20, the staining pattern looked partially vesicular (arrow) in appearance and partly associated with some cytoskeleton structures (arrowhead).

### Example 12

### Diagnosis of Osteopetrosis Using Antibodies

Proteins are isolated from a tissue sample or cultured cells (such as lymphoblasts) prepared from the osteopetrosis patient homozygous for the IVS2+1 G to A mutation, from the parents of this patient who are heterozygous for this mutation, and from an unrelated control. The isolated proteins are resolved by SDS-PAGE electrophoresis and the PLEKHM1 protein is detected by Western blotting with an anti-PLEKHM1 antibody, such as one of the anti-plekhm1A or anti-plekhm1B antibodies. The control sample reveals a band corresponding to the PLEKHM1 protein. This band is also present in the heterozygous parents, but its intensity is lower compared to the control. The band is absent in the homozygous patient. This example demonstrates the possibility to diagnose a bone-related disorder, such as osteopetrosis, by detection of the PLEKHM1 protein.

### Example 13

### Diagnosis of Osteopetrosis Using N-Terminal PLEKHM-1 Antibody

Proteins are isolated from a tissue sample or cultured cells (such as lymphoblasts) prepared from the osteopetrosis patient homozygous for the IVS2+1 G to A mutation, from the parents of this patient who are heterozygous for this mutation, and from an unrelated control. The isolated proteins are resolved by SDS-PAGE electrophoresis and the PLEKHM1 protein is detected by Western blotting with an N-terminal anti-PLEKHM1 antibody recognizing an epitope within the first 97 amino acids of the PLEKHM1 protein. The control sample reveals a band of about 117 kDa corresponding to the full-length PLEKHM1 protein. This band is absent in the homozygous patient, but another band of a lower molecular weight corresponding to the truncated PLEKHM1 protein is present. Both bands are observed in proteins isolated from the heterozygous parents. This example demonstrates the possibility to diagnose a bone-related disorder, such as osteopetrosis, by detection of truncated or modified forms of the PLEKHM1 protein.

### Summary of the Above Experimental Exemplification

In this report, a frameshift mutation in exon 4 of the plekhm1 gene was identified as responsible for the osteopetrosis phenotype in the ia rat. There had been no indication in prior art that the PLEKHM1 protein would have a role in bone tissue, such as in osteoclast metabolism. In contrast, a portion of the PLEKHM1 protein had been previously described as the AP162 protein with a putative function in colon tissue. In silico analysis of the plekhm1 protein sequence predicted the presence of 4 functional domains: a RUN domain, two PH domains and a cysteine rich domain. The presence of these domains may suggest that the PLEKHM1 protein is involved in intracellular signaling and that it may function in small GTPase (Ras-like GTPase) signaling pathways. Expression analysis of the PLEKHM1 protein using two specific anti-plekhm1 antibodies demonstrated ubiquitous expression of the plekhm1 gene including bone tissue and osteoclasts. Immuno-histochemistry on bone slides demonstrated expression in osteoclasts, osteoblasts, osteocytes, chondrocytes and megakaryocytes. The localization of the plekhm1 protein was studied using GFP technology, and immuno-fluorescence microscopy. GFP-PLEKHM1 fusion protein produced diffuse cytoplasmic distribution in HEK cells with the appearance of abnormally large vesicles. Immuno-fluorescence microscopy on rat osteoclast cultures also demonstrated plausible association of the plekhm1 protein with vesicles and cytoskeletal structures. Together this may suggest that the plekhm1 protein participates in small GTPase signaling in osteoclasts which regulates cytoskeletal reorganization and intracellular vesicular transport, i.e., processes important for osteoclast function.

Further in this report, a causative homozygous mutation in the PLEKHM1 gene (IVS2+1 G to A) was found in a human patient suffering from osteopetrosis. This mutation results in a highly truncated and therefore likely non-functional PLEKHM1 protein. This shows that the PLEKHM1 gene and protein participate in bone metabolism and bone homeostasis in humans, and that mutations and/or genetic variations in the PLEKHM1 gene and protein may disturb bone homeostasis and cause and/or contribute to development of bone-related disorders in humans, such as, e.g., osteopetrosis or osteoporosis, etc.

This shows that the PLEKHM1 gene and/or protein is useful for genetic and/or biochemical diagnosis of bone-related disorders or susceptibility thereto, in particular in humans. For example, the PLEKHM1 gene (including the coding sequence, 5′ and 3′ UTR, exons, introns, the promoter, etc.) in patients at risk of or affected with bone-related disorders may be screened for mutations and/or genetic variations by methods well-known in the art. In this respect, the inventors exemplified screening of the PLEKHM1 gene in osteopetrosis patients by sequencing of PCR products from genomic DNA and/or cDNA. In particular, the inventors exemplified a method to diagnose osteopetrosis involving the detection of the IVS2+1 G to A mutation in the PLEKHM1 gene by direct sequencing on DNA or cDNA from heterozygous and homozygous individuals and controls and by detection of size differences between PLEKHM1 RT-PCR products from heterozygous and homozygous individuals and controls. A skilled person will know the techniques to screen the PLEKHM1 gene in patients for novel or previously identified mutations and/or genetic variations (e.g., the IVS2+1 G to A mutation identified herein), including, e.g., direct sequencing, hybridisation or single-base extension on microarrays, DHPLC, pyrosequencing, SSCP, heteroduplex analysis, PCR-RFLP, allele-specific oligonucleotide (ASO) probes, single-base extension (SBE), etc. These and other techniques may use the probes and primers of the present invention. In another example, the patients at risk of or affected with bone-related disorders may be tested for the quantity of the PLEKHM1 mRNA, e.g., using quantitative RT-PCR or hybridisation with specific PLEKHM1 probes on membranes or microarrays. The skilled artisan will know to select RT-PCR primers and hybridisation probes. In another example, the patients at risk of or affected with bone-related disorders may be tested for the quantity of the PLEKHM1 protein, e.g., using specific anti-PLEKHM1 antibodies (poly- or monoclonal, etc.). The present inventors exemplified the preparation of two affinity purified polyclonal anti-PLEKHM1 antibodies. These and other anti-PLEKHM1 antibodies can specifically detect the PLEKHM1 protein. For example, quantification of the PLEKHM1 protein may be done using methods such as Western blotting, RIA or ELISA. The inventors exemplified a diagnostic method based on the detection of the PLEKHM1 protein by Western blotting in individuals with the IVS2+1 G to A mutation. If a mutation results in a truncated PLEKHM1 protein, antibodies specifically recognizing an epitope in the truncated fragment can be used to diagnose such mutation based on a different size of the full-length and truncated proteins. Further, immuno-histochemistry of the PLEKHM1 protein in (bone) tissues from patients using anti-PLEKHM1 antibodies may also be part of diagnosis, to detect, e.g., differences in the level or localization of the protein. The skilled artisan will understand that the diagnostic methods are not limited to the exemplified osteopetroses. Because the PLEKHM1 gene influences bone metabolism and homeostasis, the bone-related diseases in which diagnosis based on PLEKHM1 can be useful include various pathological condition or disease involving a disregulated bone homeostasis as described elsewhere in the specification.

Also, the present inventors demonstrated that in humans and rats, inactivation and/or reduced activity of the PLEKHM1 protein by two different mutations alters bone homeostasis, i.e., the balance between bone formation and bone resorption. In particular, inactivation and/or reduced activity of the PLEKHM1 protein inhibits bone resorption and results in increased bone mass, as evidenced by the diagnosis of osteopetrosis in ia rats and in the human patient homozygous for the IVS2+1 G to A mutation. This observation enables to modulate bone homeostasis by altering the level or activity of the PLEKHM1 protein in humans or animals. For example, decreasing the level and/or activity of the PLEKHM1 protein will reduce bone resorption and in turn enhance formation of bone tissue. Accordingly, decreasing the level and/or activity of the PLEKHM1 protein will be useful in prevention and/or treatment of diseases associated with decreased bone mass or density, such as, for example, osteoporosis. Otherwise, increasing the level and/or activity of the PLEKHM1 protein will increase bone resorption. Accordingly, increasing the level and/or activity of the PLEKHM1 protein will be beneficial in prevention and/or treatment of diseases associated with increased bone mass or density, such osteopetroses. Osteopetroses may be for example caused by impairment of the PLEKHM1 gene or by impairment of other genes involved in the bone resorption process.

### Example 14

### Therapeutic Effects of PLEKHM1 Nucleic Acids and Polypeptides

This example demonstrates the positive therapeutic effect of PLEKHM1 nucleic acids and PLEKHM1 polypeptides expressed in osteoclasts on reversal of osteopetrotic phenotype and improvement of bone resorption capacity.

Homozygous knockout (KO) mice are obtained by disrupting the PLEKHM1 gene following standard protocols in transgenic technology (such as by deleting a portion of or the entire gene). These PLEKHM1 KO mice display an osteopetrotic phenotype. These mice are further modified to express the mouse or human PLEKHM1 protein specifically in osteoclasts under a tetracycline-controlled promoter. This is achieved as follows. A recombinant construct comprising the cDNA encoding the mouse or human PLEKHM1 protein (cDNA of SEQ ID NO: 5 or 7, respectively) under the control of a promoter responsive to the tetracycline-inhibited transcription activator (tTA) is introduced to these mice by routine transgenic methods. Stable transmitting transgenic lines are established with this construct integrated into the genome. Subsequently, these mice are crossed with mice expressing the tTA under the control of a promoter which directs expression in osteoclasts. In these latter mice, continued expression of tTA may be ubiquitous or may be preferably substantially localized to osteoclasts. The resulting double transgenic mice will display tetracyclin controlled expression of the mouse or human PLEKHM1 cDNA in osteoclasts. The expression of PLEKHM1 protein can be inhibited by addition of tetracycline into the drinking water of the mice. Conversely, the expression may be stimulated when tetracyclin is omitted.

The mice which are administered tetracyclin (inhibited expression of the PLEKHM1 cDNA) develop and osteopetrotic phenotype. This phenotype is at least partially reversed in mice in which administration of tetracyclin is discontinued (stimulated expression of the PLEKHM1 cDNA). Reversal of the osteopetrotic phenotype is detected by DEXA and by measuring serum and urine bone resorption biochemical markers.

Furthermore, the bone resorption capacities of osteoclast cultures from PLEKHM1 knockout mice and wild-type mice are compared. The osteoclast cultures established from the PLEKHM1 KO mice have diminished bone resorption capacity.

This shows that upregulation of PLEKHM1 gene expression and/or provision of nucleic acid encoding PLEKHM1 or PLEKHM1 polypeptides can be used to prevent or treat bone-related disease characterised by diminished bone resorption, such as osteopetroses.

### Example 15

### Downregulation or Absence of the PLEKHM1 Gene Counteracts Bone Loss

This example demonstrates the beneficial effect of downregulation or absence of the the PLEKHM1 gene and/or protein on counteracting bone loss in animal model. Ovaryectomy of rats typically results in bone loss. Ovaryectomy is performed in ia rats and in wild-type controls. DEXA scanning of bone mineral density at different time points demonstrates that the bone loss is less pronounced in the ia rat group than in the wild-type group. This shows that therapeutic downregulation of the PLEKHM1 gene and/or inhibition of the PLEKHM1 protein can be used to prevent or treat bone-related diseases characterised by bone loss, such as osteoporosis and cancer-induced bone loss.

### Example 16

### PLEKHM1 Affects Bone Resorption, Assay for PLEKHM1 Modulators

This example demonstrates the effect of the level of PLEKHM1 protein on the bone resorption capacity of osteoclasts in vitro. This cell-based in vitro assay is also useful for screening for compounds which can modulate bone resorption capacity of osteoclasts by altering the levels, activity or function of PLEKHM1 or pathways in which it is involved.

Osteoclasts are prepared from ia rats, wild-type rats and transgenic rats overexpressing PLEKHM1 or alternatively from PLEKHM1 KO mice, wild-type mice and transgenic mice overexpressing PLEKHM1. In particular, osteoclasts are derived from bone marrow progenitor cells of these animals which are differentiated into osteoclasts by the administration of RANKL and MCSF. These osteoclasts are used to perform in vitro bone resorption assays, wherein the bone resorption capacity is assessed by measuring the dimensions of resorption pits (such as depth or total area) produced by these osteoclasts cultured on bone slices. Compared to osteoclasts from wild-type mice, resorption capacity is diminished in osteoclasts from PLEKHM1 KO mice and is increased in osteoclasts from mice over expressing PLEKHM1. This illustrates the effect of PLEKHM1 for bone resorption.

The above assay is used to identify compounds, such as peptides or small molecule compounds, which can modulate the bone resorption capacity of osteoclasts via PLEKHM1 or its pathways. In particular, the assay is first performed using osteoclasts from wild-type mice. The bone resorption capacity of wt osteoclasts is tested in the presence and absence of a test compound. If the test compound modulates, e.g., increases or decreases, the bone resorption capacity of wild-type osteoclasts, it is then tested in an assay involving osteoclasts from PLEKHM1 KO mice. If the effect of the test compound in PLEKHM1 KO osteoclasts is reduced or abolished compared to wt osteoclasts, the test compound influences bone resorption via PLEKHM1 or its pathway.

The identified test compounds may increase or decrease the bone resorption capacity of osteoclasts. Therefore, such compounds represent therapeutic candidates for treatment or prevention of bone-related diseases characterised by increased (e.g., osteopetroses, sclerosing bone dysplasias) or decreased (e.g., osteoporosis, Paget's disease, cancer-induced bone loss, bone loss in osteoarthritis and reumatoid arthritis) bone mass, respectively.

### Example 17

### Inhibition of PLEKHM1

Polyclonal antibody against PLEKHM1 is administered to wild-type rats and ovaryectomized rats. In both groups, bone mineral density is compared between the animals which received the antibody and those that didn't. Bone mineral density is increased in the animals that received the antibody.

This suggests that downregulation or inhibition of PLEKHM1, such as by antibodies (other agents are possible, e.g., antisense nucleic acids, small interfering RNA, peptides, small molecules, etc. . . . ) can be used to increase bone mineral density early in life and/or to prevent or treat bone loss (e.g., osteoporosis herein modeled by ovaryectomy) later in life. The above example may also advantageously employ monoclonal antibodies.

The delivery of the PLEKHM1 inhibitor may be for example local, such as by injection or surgical implantation, e.g., to prevent local bone loss, such as around the teeth in periodontal disease, or at specific sites of inflammation-induced bone loss, for example in rheumatoid arthritis.

### Example 18

### Screening for Molecules Binding to PLEKHM1

A PLEKHM1 expression construct making a flagged PLEKHM1 fusion protein is transfected and isolated over a column. The isolated protein is used in a cell free screening assay for test compounds binding to the protein. Binding is detected by standard methods, e.g., immunodetection. The test compounds may involve different classes, such as peptides and small organic molecules—libraries of such compounds are available. Test compounds which bind to PLEKHM1 protein represent putative drug leads for the treatment of bone-related disorders of impaired bone homeostasis, such as Paget's disease and osteoporosis and the prevention of cancer-induced bone loss and bone loss in osteoarthritis and reumatoid arthritis.

In an alternative assay, the effect of PLEKHM1 protein on prenylation of rabs in assayed in vitro. Test compounds are identified which can modulate, e.g., increase or decrease, this effect. The test compounds may involve different classes, such as peptides and small organic molecules—libraries of such compounds are available. Test compounds which modulate the effect of PLEKHM1 on prenylation of rabs represent putative drug leads for the treatment of bone-related disorders of impaired bone homeostasis, such as Paget's disease and osteoporosis and the prevention of cancer-induced bone loss and bone loss in osteoarthritis and reumatoid arthritis.

1.-34. (canceled)

35. A method of diagnosing osteopetrosis or susceptibility to osteopetrosis in a subject, the method comprising:
providing a sample comprising nucleic acid from the subject, and
detecting one or more of:
(i) the presence or absence of a mutation in a nucleic acid encoding a mammalian PLEKHM1 polypeptide or the complement thereof;
(ii) the presence or amount of a nucleic acid encoding a mammalian PLEKHM1 polypeptide or the complement thereof; or
(iii) the expression or amount of a mammalian PLEKHM1 polypeptide, in the sample;
wherein the presence or absence of a mutation, the presence or amount of the nucleic acid, or the presence or amount of the polypeptide indicate whether the subject has, or is susceptible to, osteopetrosis.

36. The method according to claim 35, wherein said nucleic acid comprises SEQ ID NO:1, 3, 5, 7, 9, or 10, or the complement thereof, or wherein said polypeptide comprises the amino acid sequence of SEQ ID NO:2, 4, 6, 8, or 11, or a variant or derivative thereof, or an immunologically active and/or functional fragment thereof.

37. The method according to claim 35, wherein the subject is human, and the method comprises detecting one or more of:
(i) the presence or absence of a mutation at position +1 of the donor splice site of intron 2 of the nucleic acid encoding human PLEKHM1 polypeptide, or the complement thereof;
(ii) the presence or amount of a truncated mRNA encoding human PLEKHM1 polypeptide, or the complement thereof; or
(iii) detecting expression or amount of a truncated mammalian PLEKHM1 polypeptide, in the sample.

38. A kit for diagnosing osteopetrosis or a predisposition to osteopetrosis, comprising one or both of a primer or a probe for detecting the presence of a mutation in a nucleic acid encoding a mammalian PLEKHM1 polypeptide or the complement thereof in a sample, or for determining the presence or amount of a nucleic acid encoding a mutant mammalian PLEKHM1 polypeptide or the complement thereof in a sample.

39. The kit according to claim 37, wherein said nucleic acid comprises SEQ ID NO:1, 3, 5, 7, 9 or 10, or the complement thereof.

40. The kit according to claim 37, wherein said primer or probe is at least 15 nucleotides in length, specifically hybridizes to a nucleotide sequence of SEQ ID NO:1, 3, 5, 7, 9, or 10, or the complement thereof, and is capable of specifically amplifying a nucleic acid comprising SEQ ID NO:1, 3, 5, 7, 9, or 10, or a portion thereof.

41. A kit for diagnosing osteopetrosis or a predisposition to osteopetrosis, comprising an antibody or antigen-binding fragment thereof that specifically recognizes a mammalian PLEKHM1 polypeptide.

42. The kit according to claim 40, wherein said polypeptide comprises the amino acid sequence of SEQ ID NO:2, 4, 6, 8, or 11.

43. A method of preventing, treating, or alleviating osteopetrosis, the method comprising administering a therapeutically effective amount of a nucleic acid encoding a mammalian PLEKHM1 polypeptide or functional variant or fragment thereof, or the complement thereof, to a subject in need thereof.

44. The method of claim 42, wherein the osteopetrosis is autosomal-recessive osteopetrosis.

45. The method of claim 42, wherein said nucleic acid comprises SEQ ID NO:1, SEQ ID NO:5, or SEQ ID NO:7, or the complement thereof.

46. The method of claim 45, wherein said nucleic acid comprises a nucleotide sequence that is at least 95% identical to SEQ ID NO:1, 3, 5, or 7, or the complement thereof.

47. The method of claim 42, wherein said nucleic acid specifically hybridizes to the nucleotide sequence of SEQ ID NO:1, 3, 5, or 7, or the complement thereof.

48. A method of preventing, treating, or alleviating osteopetrosis, the method comprising administering a therapeutically effective amount of a mammalian PLEKHM1 polypeptide or functional variant or fragment thereof to a subject in need thereof.

49. The method of claim 48, wherein said isolated polypeptide comprises the amino add sequence of SEQ ID NO:2, 6, or 8, or a biologically active variant or derivative thereof or functional fragment thereof.

50. A method of preventing, treating, or alleviating osteopetrosis, the method comprising administering a therapeutically effective amount of bone marrow cells expressing a functional exogenous mammalian PLEKHM1 nucleic acid to a subject in need thereof.

51. The method of claim 50, wherein the osteopetrosis is autosomal-recessive osteopetrosis.
